(19) United States
(12) Patent Application Publication
(10) Pub. No.: US 2014/0374733 A1
(43) Pub. Date: Dec. 25, 2014

(54) PHOTOELECTRIC CONVERSION ELEMENT, METHOD FOR USING THE SAME, IMAGING DEVICE, PHOTOSENSOR, AND COMPOUND

(71) Applicant: FUJIFILM Corporation, Tokyo (JP)

(72) Inventors: Yuki HIRAI, Kanagawa (JP); Yosuke YAMAMOTO, Kanagawa (JP); Tomoaki YOSHIOKA, Kanagawa (JP); Eiji FUKUZAKI, Kanagawa (JP); Katsuyuki YOFU, Kanagawa (JP); Masaaki TSUKASE, Kanagawa (JP); Mitsumasa HAMANO, Kanagawa (JP); Takahiko ICHIKI, Kanagawa (JP)

(73) Assignee: FUJIFILM Corporation, Tokyo (JP)

(21) Appl. No.: 14/479,063

(22) Filed: Sep. 5, 2014

Related U.S. Application Data
(63) Continuation of application No. PCT/JP2013/055862, filed on Mar. 4, 2013.

(30) Foreign Application Priority Data
Mar. 5, 2012 (JP) 2012-047889

(51) Int. Cl. H01L 51/00

(52) U.S. Cl. CPC H01L 51/0072; H01L 51/0046; H01L 51/001; H01L 51/442
USPC 257/40; 546/61; 546/42; 546/70; 546/62; 544/102; 546/38; 546/58

(57) ABSTRACT

An object of the present invention is to provide a photoelectric conversion element having a photoelectric conversion film which exhibits heat resistance, a high photoelectric conversion efficiency, a low level of dark currents, rapid response, and sensitivity characteristics to red and can be produced by a vapor deposition processing that is continuously performed under a high-temperature condition. The photoelectric conversion element of the present invention is a photoelectric conversion element in which a conductive film, a photoelectric conversion film containing a photoelectric conversion material, and a transparent conductive film are laminated on one another in this order, wherein the photoelectric conversion material includes a compound represented by Formula (1).

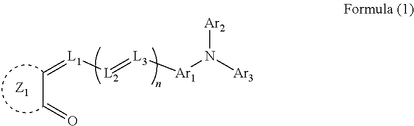

### CROSS-REFERENCE TO RELATED APPLICATIONS

This application is a Continuation of PCT International Application No. PCT/JP2013/055862 filed on Mar. 4, 2013, which claims priority under 35 U.S.C. §119(a) to Japanese Application No. 2012-047889 filed on Mar. 5, 2012. The above application is hereby expressly incorporated by reference, in its entirety, into the present application.

### BACKGROUND OF THE INVENTION

This invention relates to a photoelectric conversion element, a method for using the photoelectric conversion element, an imaging device, a photosensor, and a compound.

A conventional photosensor is an element in which a photodiode (PD) is formed in a semiconductor substrate such as silicon (Si), and as a solid-state imaging device, a planar solid-state imaging device, in which PDs are two-dimensionally arranged and signal charge generated by each PD is read out by a circuit, is widely used.

In order to obtain a color solid-state imaging device, a structure in which color filters that transmit light of a specific wavelength are arranged in a light incident surface of the planar solid-state imaging device is generally used. Currently, a single plate solid-state imaging device, in which color filters that transmit blue (B) light, green (G) light, and red (R) light are regularly arranged on each of the two-dimensionally arranged PDs, is well known and is widely used in a digital camera and the like.

In the single plate solid-state imaging device, the light not transmitted through the color filters is not utilized, and accordingly, light use efficiency is poor. Moreover, in recent years, the number of pixels has been increased, and the pixel size has been reduced. Consequentially, decrease in aperture ratio and decrease in light-collecting efficiency have become problems.

As solutions to the above problems, a structure obtained by forming a photoelectric conversion film formed of amorphous silicon or an organic photoelectric conversion film on a substrate for reading out signal is known.

Regarding the photoelectric conversion element, imaging device, and photosensor using an organic photoelectric conversion film, several prior art exist. In the photoelectric conversion element using an organic photoelectric conversion film, particularly, the improvement of photoelectric conversion efficiency and reduction of dark current become problems. As solutions to the problems, formation of a pn junction or a bulk-heterostructure has been suggested for the former problem, and formation of a blocking layer or the like has been suggested for the latter problem.

For example, JP 2011-213706 A discloses a photoelectric conversion film containing, for example, a compound represented by the following formula in the section of example. The document describes that the photoelectric conversion film has a high photoelectric conversion efficiency.

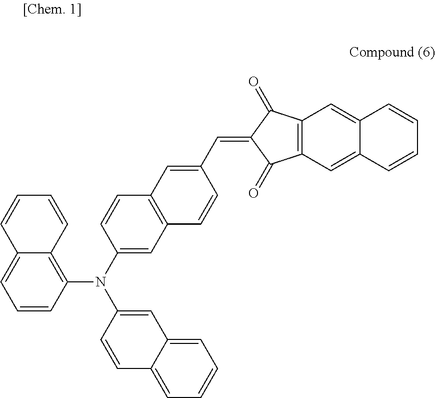 [SMILES: O=C1C(=Cc2ccc3cc(N(c4ccc5ccccc5c4)c4cccc5ccccc45)ccc3c2)C(=O)c2cc3ccccc3cc21]

### SUMMARY OF THE INVENTION

Generally, for producing a photoelectric conversion element using an organic photoelectric conversion film, a photoelectric conversion dye is subjected to a vapor deposition processing under a high-temperature condition in many cases. Considering productivity of the photoelectric conversion element, it is desirable to continuously perform the vapor deposition processing over a long time at a high vapor deposition speed.

Meanwhile, the present inventors evaluate vapor deposition characteristics of a specific compound described in the section of example of JP 2011-213706 A. As a result, it was found that if the temperature is further increased so as to increase the vapor deposition speed, the compound is degraded, purity of the formed photoelectric conversion film deteriorates, and consequentially, it is difficult to continuously perform the vapor deposition processing in some cases.

Moreover, generally, it is desirable for the photoelectric conversion element to have spectral sensitivity in the entire visible light region, and particularly, the element is required to have high sensitivity in a red light region. However, in the conventional techniques, even though the compound has excellent vapor deposition characteristics, the spectral characteristics of the photoelectric conversion element are poor.

Furthermore, in recent years, imaging devices, photosensors, and the like have been required to be improved in terms of the performance, and accordingly, improvement of various characteristics (heat resistance, photoelectric conversion efficiency, dark currents, response speed, and the like) required for the photoelectric conversion film used for them have also been required.

The present inventors prepared a photoelectric conversion film by using the compound (for example, the compound 6) specifically disclosed in detail in JP 2011-213706 A. As a result, they found that in terms of heat resistance, photoelectric conversion efficiency, response speed, or occurrence of dark currents, the photoelectric conversion film has not reached a currently required level and needs to be further improved.

As described above, in the conventional techniques, it is difficult to establish both the productivity of a photoelectric conversion element produced by vapor deposition and various characteristics (heat resistance, photoelectric conversion efficiency, dark currents, responsiveness, and optical characteristics) of a photoelectric conversion film.

The present invention has been made in consideration of the above circumstances, and an object thereof is to provide a photoelectric conversion element having a photoelectric conversion film which exhibits heat resistance, a high photoelectric conversion efficiency, a low level of dark currents, rapid response, and sensitivity characteristics to red and can be produced by a vapor deposition processing that is continuously performed under a high-temperature condition.

Another object of the present invention is to provide a method for using the photoelectric conversion element and to provide an imaging device and a photosensor including the photoelectric conversion element.

As a result of thorough examination to achieve the above objects, the present inventors found that by introducing a halogen group or a halogenated alkyl group and also by introducing a condensed ring structure into a predetermined position of a compound contained in a photoelectric conversion film, the above objects can be achieved, and thus accomplished the present invention.

That is, the above objects can be achieved by the following means.

(1) A photoelectric conversion element in which a conductive film, a photoelectric conversion film containing a photoelectric conversion material, and a transparent conductive film are laminated on one another in this order, wherein the photoelectric conversion material includes a compound represented by Formula (1) which will be described later.

(2) The photoelectric conversion element according to (1), wherein Z₁ is a group represented by Formula (Z1) which will be described later.

(3) The photoelectric conversion element according to (1) or (2), wherein Ar₁ is a substituted or unsubstituted divalent arylene group.

(4) The photoelectric conversion element according to any one of (1) to (3), wherein the halogen group or a halogen group contained in the halogenated alkyl group is a chloro group or a fluorine group.

(5) The photoelectric conversion element according to any one of (1) to (4), wherein at least one of Z₁, Ar₁, Ar₂, and Ar₃ is substituted with a halogen group.

(6) The photoelectric conversion element according to any one of (1) to (5), wherein at least one of Z₁, Ar₂, and Ar₃ is substituted with one to two halogen groups or halogenated alkyl groups.

(7) The photoelectric conversion element according to any one of (1) to (6), wherein the compound represented by Formula (1) is a compound represented by Formula (2) which will be described later.

(8) The photoelectric conversion element according to (7), wherein at least one of Ar₂₂ and Ar₃ in Formula (2) is substituted with a halogen group.

(9) The photoelectric conversion element according to (8), wherein the halogen group is a fluorine group.

(10) The photoelectric conversion element according to any one of (1) to (9), wherein the photoelectric conversion film further contains an n-type organic compound.

(11) The photoelectric conversion element according to (10), wherein the n-type organic compound contains fullerene or derivatives thereof.

(12) The photoelectric conversion element according to (11), wherein a ratio of the content of the fullerene or derivatives thereof to a total content of the fullerene or derivatives thereof and the compound represented by Formula (1) (a film thickness expressed in terms of a single layer of the fullerene or derivatives thereof/(a film thickness expressed in terms of a single layer of the compound represented by Formula (1)+a film thickness expressed in terms of a single layer of the fullerene or derivatives thereof)) is equal to or higher than 50% by volume.

(13) The photoelectric conversion element according to any one of (1) to (12), wherein a charge blocking layer is disposed between the conductive film and the transparent conductive film.

(14) The photoelectric conversion element according to any one of (1) to (13), wherein the photoelectric conversion film is formed by a vacuum vapor deposition method.

(15) The photoelectric conversion element according to any one of (1) to (14), wherein light enters the photoelectric conversion film through the transparent conductive film.

(16) The photoelectric conversion element according to any one of (1) to (15), wherein the transparent conductive film is formed of a transparent conductive metal oxide.

(17) An imaging device comprising the photoelectric conversion element according to any one of (1) to (16).

(18) A photosensor comprising the photoelectric conversion element according to any one of (1) to (16).

(19) A method for using the photoelectric conversion element according to any one of (1) to (16), wherein the conductive film and the transparent conductive film form a pair of electrodes, and an electric field of 1×10⁻⁴ V/cm to 1×10⁷ V/cm is applied between the pair of electrodes.

(20) A compound represented by Formula (1) which will be described later

According to the present invention, it is possible to provide a photoelectric conversion element having a photoelectric conversion film which exhibits heat resistance, a high photoelectric conversion efficiency, a low level of dark current, rapid response, and sensitivity characteristics to red and can be produced by a vapor deposition processing that is continuously performed under a high-temperature condition.

Moreover, according to the present invention, it is possible to provide a method for using the photoelectric conversion element and an imaging device and a photosensor including the photoelectric conversion element.

### BRIEF DESCRIPTION OF THE DRAWINGS

Each of FIG. 1A and FIG. 1B is a schematic cross-sectional view showing an example of configuration of a photoelectric conversion element.

FIG. 2 is a schematic cross-sectional view of one pixel of an imaging device.

### DETAILED DESCRIPTION OF THE INVENTION

Hereinafter, preferable embodiments of the photoelectric conversion element and the method for using the element of the present invention will be described.

First, characteristics of the present invention will be described in detail through comparison between the present invention and the conventional techniques.

As described above, in the present invention, it was found that by introducing a halogen group or a halogenated alkyl group and also by introducing a condensed ring structure into a predetermined position of a compound contained in a photoelectric conversion film, intended effects are obtained. Particularly, in the conventional techniques, it is known that durability of a voltage-driven element represented by an organic EL greatly deteriorates when halogen (particularly, chlorine) is mixed into the element (for example, JP 2005-68263 A, JP 2004-327455 A, and the like). On the contrary, in the present invention, it was found that unlike the knowledge of conventional techniques, by introducing a halogen group or a halogenated alkyl group into a compound, it is possible to improve vapor deposition characteristics of the compound without deteriorating various characteristics including durability. Presumably, this is because due to the introduction of a halogen group or a halogenated alkyl group, interaction between molecules of the compound may be weakened; sublimation properties may be improved; a degree of freedom of the compound may decrease; a melting point may be heightened; and as a result, vapor deposition characteristics (heat resistance) may be improved. Furthermore, presumably, due to the introduction of a condensed ring structure, vapor deposition characteristics may be further improved, and various characteristics of the photoelectric conversion element may also be improved.

Hereinafter, preferable embodiments of the photoelectric conversion element of the present invention will be described with reference of drawings. FIG. 1 is a schematic cross-sectional view of an embodiment of the photoelectric conversion element of the present invention.

A photoelectric conversion element 10a shown in FIG. 1A has a configuration in which a conductive film 11 (hereinafter, also referred to as a “lower electrode”) that functions as a lower electrode, an electron blocking layer 16A that is formed on the lower electrode 11, a photoelectric conversion layer 12 that is formed on the electron blocking layer 16A, and a transparent conductive film 15 (hereinafter, also referred to as a “upper electrode”) that functions as an upper electrode are laminated on one another in this order.

FIG. 1B shows an example of configuration of another photoelectric conversion element. A photoelectric conversion element 10b shown in FIG. 1B has a configuration in which on the lower electrode 11, the electron blocking layer 16A, the photoelectric conversion layer 12, a hole blocking layer 16B, and the upper electrode 15 are laminated in this order. The electron blocking layer 16A, the photoelectric conversion layer 12, and the hole blocking layer 16B in FIG. 1A and FIG. 1B may be laminated in an inverse order, according to the use and characteristics of the element. For example, the position of the electron blocking layer 16A and the photoelectric conversion layer 12 may be switched.

In the present specification, the photoelectric conversion film may include at least a photoelectric conversion layer and further include a charge blocking layer (an electron blocking layer or a hole blocking layer).

In the configuration of the photoelectric conversion element 10a (10b), it is preferable for light to enter the photoelectric conversion layer 12 through the transparent conductive film 15.

Furthermore, when the photoelectric conversion element 10a (10b) is used, an electric field can be applied thereto. In this case, the conductive film 11 and the transparent conductive film 15 form a pair of electrodes, and it is preferable to apply an electric field of 1×10⁻⁵ V/cm to 1×10⁷ V/cm between the pair of electrodes. From the viewpoint of performance and power consumption, an electric field of 1×10⁻⁴ V/cm to 1×10⁶ V/cm is preferable, and an electric field of 1×10⁻³ V/cm to 5×10⁵ V/cm is particularly preferable.

Regarding a voltage applying method, it is preferable to apply voltage such that the electron blocking layer 16A becomes a negative pole, and the photoelectric conversion layer 12 becomes a positive pole, in FIG. 1A and FIG. 1B. When the photoelectric conversion element 10a (10b) is used as a photosensor or included in an imaging device, voltage can be applied by the same method as described above.

Hereinafter, embodiments of the respective layers (photoelectric conversion layer 12, electron blocking layer 16A, lower electrode 11, upper electrode 15, hole blocking layer 16B, and the like) configuring the photoelectric conversion element 10a (10b) will be described in detail.

First, the photoelectric conversion layer 12 will be described in detail.

[Photoelectric Conversion Layer]

The photoelectric conversion layer 12 is a film that contains, as a photoelectric conversion material, a compound represented by Formula (1) which will be described later. If such a compound is used, it is possible to obtain a photoelectric conversion layer, which exhibits heat resistance, a high photoelectric conversion efficiency, a low level of dark currents, rapid response, and sensitivity characteristics to red, with excellent productivity while maintaining a high vapor deposition rate, by a vapor deposition processing conducted under a high-temperature condition.

First, the compound used for the photoelectric conversion layer 12 and represented by Formula (1) will be described in detail. The compound represented by Formula (1) has a structure having a donor site (site of —Ar₁NAr₂Ar₃) and an acceptor site (site of ═Z₁O).

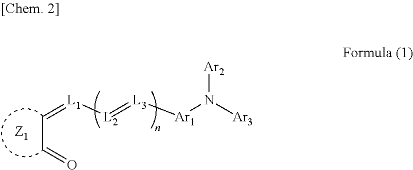

In Formula (1), Z₁ is a ring that contains at least two carbon atoms and may have a substituent, and represents a 5-membered ring, a 6-membered ring, or a condensed ring which contains at least one of 5-membered ring and 6-membered ring. As the 5-membered ring, the 6-membered ring, or the condensed ring which contains at least one of 5-membered ring and 6-membered ring, rings which are usually used as an acidic nucleus in merocyanine dyes are preferable, and specific examples thereof include the following.

(a) 1,3-Dicarbonyl nuclei: for example, a 1,3-indandione nucleus, 1,3-cyclohexanedione, 5,5-dimethyl-1,3-cyclohexanedione, and 1,3-dioxane-4,6-dione

(b) Pyrazolinone nuclei: for example, 1-phenyl-2-pyrazolin-5-one, 3-methyl-1-phenyl-2-pyrazolin-5-one, and 1-(2-benzothiazolyl)-3-methyl-2-pyrazolin-5-one

(c) Isoxazolinone nuceli: for example, 3-phenyl-2-isoxazolin-5-one, and 3-methyl-2-isoxazolin-5-one

(d) Oxindole nuclei: for example, 1-alkyl-2,3-hydro-2-oxindole

(e) 2,4,6-Triketohexahydropyrimidine nuclei: for example, barbituric acid or 2-thibarbituric acid and derivatives thereof; Examples of the derivatives include 1-alkyl compounds such as 1-methyl and 1-ethyl, 1,3-dialkyl compounds such as 1,3-dimethyl, 1,3-diethyl, and 1,3-dibutyl, 1,3-diaryl compounds such as 1,3-diphenyl, 1,3-di(p-chlorophenyl), and 1,3-di(p-ethoxycarbonylphenyl), 1-alkyl-1-aryl compounds such as 1-ethyl-3-phenyl, 1,3-position diheterocyclic compounds such as 1,3-di(2-pyridyl), and the like.

(f) 2-Thio-2,4-thiazolidinedione nuclei: for example, rhodanine and derivatives thereof; Examples of the substituents include 3-aklylrhodanine such as 3-methylrhodanome, 3-ethylrhodanine, and 3-allylrhodanine, 3-arylrhodanine such as 3-phenylrhodanine, 3-position heterocyclic rhodanine such as 3-(2-pyridyl)rhodanine, and the like.

(g) 2-Thio-2,4-oxazolidinedione (2-thio-2,4-(3H,5H)-oxazoledione) nuclei: for example, 3-ethyl-2-thio-2,4-oxazolidinedione

(h) Thianaphthenone nuclei: for example, 3(2H)-thianaphthenone-1,1-dioxide

(i) 2-Thio-2,5-thizolidinedione nuclei: for example, 3-ethyl-2-thio-2,5-thiazolidinedione

(j) 2,4-Thiazolidinedione nuclei: for example, 2,4-thiazolidinedione, 3-ethyl-2,4-thiazolidinedione, and 3-phenyl-2,4-thiazolidinedione

(k) Thiazoliin-4-one nuclei: for example, 4-thiazolinone and 2-ethyl-4-thiazolinone

(l) 2,4-Imidazolidinedione (hydantoin) nuclei: for example, 2,4-imidazolidinedione and 3-ethyl-2,4-imidazolidinedione

(m) 2-Thio-2,4-imidazolidinedione (2-thiohydantoin) nuclei: for example, 2-thio-2,4-imidazolidinedione and 3-ethyl-2-thio-2,4-imidazolidinedione

(n) Imidazolin-5-one nuclei: for example, 2-propylmercapto-2-imidazolin-5-one

(o) 3,5-Pyrazolidinedione nuclei: for example, 1,2-diphenyl-3,5-pyrazolidinedone and 1,2-dimethyl-3,5-pyrazolidinedone

(p) Benzothiophen-3-one nuclei: for example, benzothiophen-3-one, oxobenzothiophen-3-one, and dioxobenzothiophen-3-one

(q) Indanone nuclei: for example, 1-indanone, 3-phenyl-1-indanone, 3-methyl-1-indanone, 3,3-diphenyl-1-indanone, and 3,3-dimethyl-1-indanone

As the ring formed by Z₁, 1,3-dicarbonyl nuclei, pyrazolinone nuclei, 2,4,6-triketohexahydropyrimidine nuclei (including thioketone compounds such as barbituric acid nuclei and 2-thiobarbituric acid nuclei), 2-thio-2,4-thiazolidinedione nuclei, 2-thio-2,4-oxazolidinedione nuclei, 2-thio-2,5-thiazolidinedione nuclei, 2,4-thiazolidinedione nuclei, 2,4-imidazolidinedione nuclei, 2-thio-2,4-imidazolidinedione nuclei, 2-imidazolin-5-one nuclei, 3,5-pyrazolidinedione nuclei, benzothiophen-3-one nuclei, and indanone are preferable; 1,3-dicarbonyl nuclei, 2,4,6-diketohexahydropyrimidine nuclei (including thiketone compounds such as barbituric acid nuclei and 2-thiobarbituric acid nuclei), 3,5-pyrazolidinedione nuclei, benzothiophen-3-one nuclei, and indanone nuclei are more preferable; 1,3-dicarbonyl nuclei and 2,4,6-triketohexahydropyrimidine nuclei (including thiketone compounds such as barbituric acid nuclei and 2-thiobarbituric acid nuclei) are even more preferable; and 1,3-indandione nuclei, barbituric acid nuclei, 2-thiobarbituric acid nuclei, and derivatives thereof are particularly preferable.

When Z₁ has a substituent, examples of the substituent include a substituent W which will be described later.

The ring represented by Z₁ is preferably represented by the following Formula (Z1), since various characteristics of the photoelectric conversion element, such as sensitivity to red, becomes excellent. * represents a binding site where the ring is bonded to L₁.

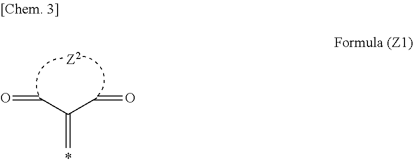

Z₂ is a ring that contains at least two carbon atoms and represents a 5-membered ring, a 6-membered ring, or a condensed ring which contains at least one of 5-membered ring and 6-membered ring. * represents a binding site where the ring is bonded to L₁ in Formula (1).

Z₂ can be selected from rings formed by Z₁. Z₂ is preferably a 1,3-dicarbonyl nucleus or a 2,4,6-triketohexahydropyrimidine nucleus (including thioketone analogues), and particularly preferably a 1,3-indanedione nucleus, a barbituric acid nucleus, a 2-thiobarbituric acid nucleus, or derivatives of these.

In Formula (1), the ring represented by Z₁ mainly functions as an acceptor site. The present inventors found that if interaction between the acceptor sites is controlled, when a film is obtained by vapor-depositing the compound together with fullerene C₆₀, the film can exhibit excellent charge transporting ability. The interaction can be controlled by introducing the structure of acceptor site and a substituent which becomes steric hindrance. In the barbituric acid nucleus and 2-thiobarbituric acid nucleus, if two hydrogen atoms in two N-positions are substituted with a substituent, the intermolecular interaction can be preferably controlled. Examples of the substituent include the substituent W which will be described later. The substituent is more preferably an alkyl group, and even more preferably a methyl group, an ethyl group, a propyl group, or a butyl group.

When the ring represented by Z₁ is a 1,3-indanedione nucleus, the ring is preferably a group represented by the following Formula (Z2) or (Z3), since various characteristics of the photoelectric conversion element, such as sensitivity to red, are further improved. * represents a binding site where the ring is bonded to L₁.

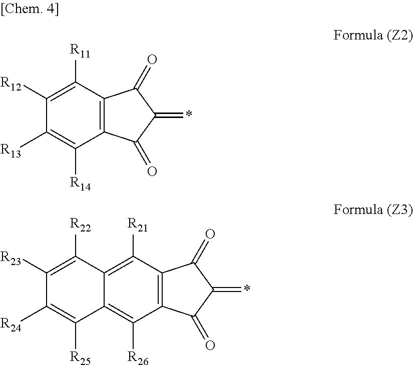

In the group represented by Formula (Z2), each of R₁₁ to R₁₄ independently represents a hydrogen atom or a substituent. As the substituent, for example, those exemplified as the substituent W, which will be described later, can be used. The substituent is preferably a halogen atom, a halogenated alkyl group, an alkyl group, or a hydrogen atom, and more preferably a halogen atom or a halogenated alkyl group. Moreover, among R₁₁ to R₁₄, two adjacent substituents may form a ring by being bonded to each other. When the ring is formed, it is preferable for R₁₂ and R₁₃ to form a ring (for example, a benzene ring, a pyridine ring, or a pyrazine ring) by being bonded to each other.

Moreover, when a halogen group or a halogenated alkyl group is introduced into the group represented by Formula (Z2), at least one of R₁₁ to R₁₄ is a halogen group or a halogenated alkyl group. Alternatively, at least one of R₁₁ to R₁₄ is a group having a substituent (for example, an alkyl group having a substituent or an aryl group having a substituent), and the substituent is a halogen group or a halogenated alkyl group.

In the group represented by Formula (Z3), each of R₂₁ to R₂₆ independently represents a hydrogen atom or a substituent. As the substituent, those exemplified as the substituent W which will be described later can be used. The substituent is preferably a halogen atom, a halogenated alkyl group, an alkyl group, or a hydrogen atom, and more preferably a halogen atom, a halogenated alkyl group, or a hydrogen atom.

Moreover, when a halogen group or a halogenated alkyl group is introduced into the group represented by Formula (Z3), at least one of R₂₁ to R₂₆ is a halogen group or a halogenated alkyl group. Alternatively, at least one of R₂₁ to R₂₆ is a group having a substituent (for example, an alkyl group having a substituent or an aryl group having a substituent), and the substituent is a halogen group or a halogenated alkyl group.

When the ring represented by Z₁ is a 2,4,6-triketohexahydropyrimidine nucleus (including thioketone analogues), the ring is preferably a group represented by Formula (Z4). * represents a binding site where the ring is bonded to L₁.

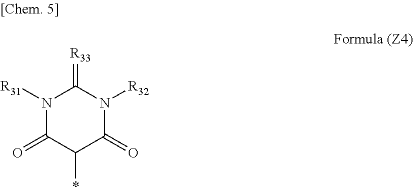

In Formula (Z4), each of R₃₁ and R₃₂ independently represents a hydrogen atom or a substituent. As the substituent, for example, those exemplified as the substituent W, which will be described later, can be used. Each of R₃₁ and R₃₂ preferably independently represents an alkyl group, an aryl group, or a heterocyclic group (preferably a 2-pyridyl or the like), and more preferably independently represents an alkyl group having 1 to 6 carbon atoms (for example, methyl, ethyl, n-propyl, or t-butyl).

Moreover, when a halogen group or a halogenated alkyl group is introduced into the group represented by Formula (Z4), at least one of R₃₁ and R₃₂ is a halogen group or a halogenated alkyl group. Alternatively, at least one of R₃₁ and R₃₂ is a group having a substituent (for example, an alkyl group having a substituent or an aryl group having a substituent), and the substituent is a halogen group or a halogenated alkyl group.

R₃₃ represents an oxygen atom, a sulfur atom, or a substituent, but preferably represents an oxygen atom or a sulfur atom. As the substituent, substituents having a nitrogen atom as a binding portion and substituents having a carbon atom as a binding portion are preferable. In the case of nitrogen atom, the substituent is preferably an alkyl group (having 1 to 12 carbon atoms) or an aryl group (having 6 to 12), and specific examples thereof include a methylamino group, an ethylamino group, a butylamino group, a hexylamino group, a phenylamino group, and a naphthylamino group. In the case of carbon atom, it is preferable for at least one electron-attracting group to be further substituted, and examples of the electron-attracting group include a carbonyl group, a cyano group, a sulfoxide group, a sulfonyl group, and a phosphoryl group. These may be further have a substituent, and examples of the substituent include the substituent W which will be described later. As R₃₃, groups that contain carbon atoms and form a 5-membered ring or a 6-membered ring are preferable, and specific examples thereof include groups having the following structure.

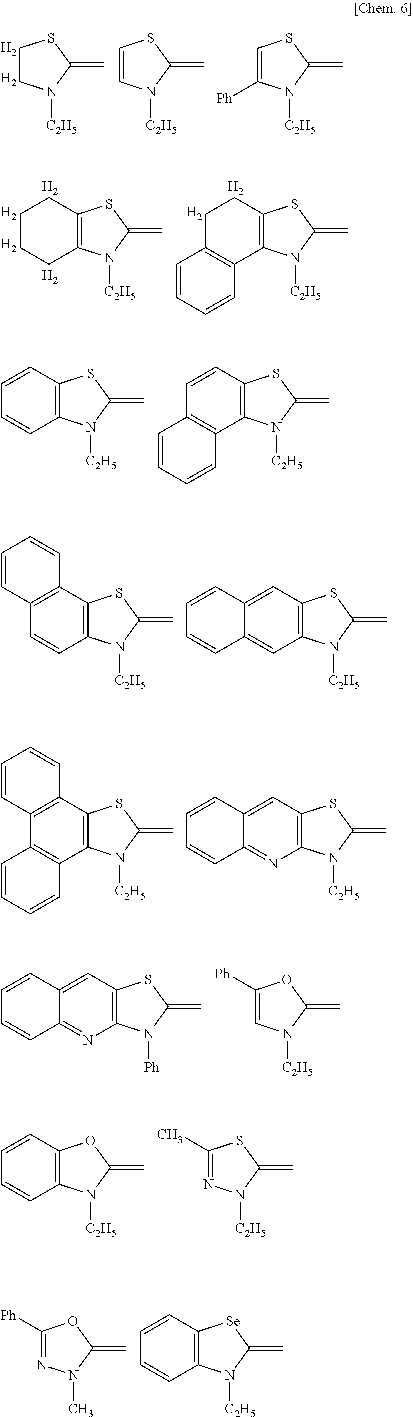

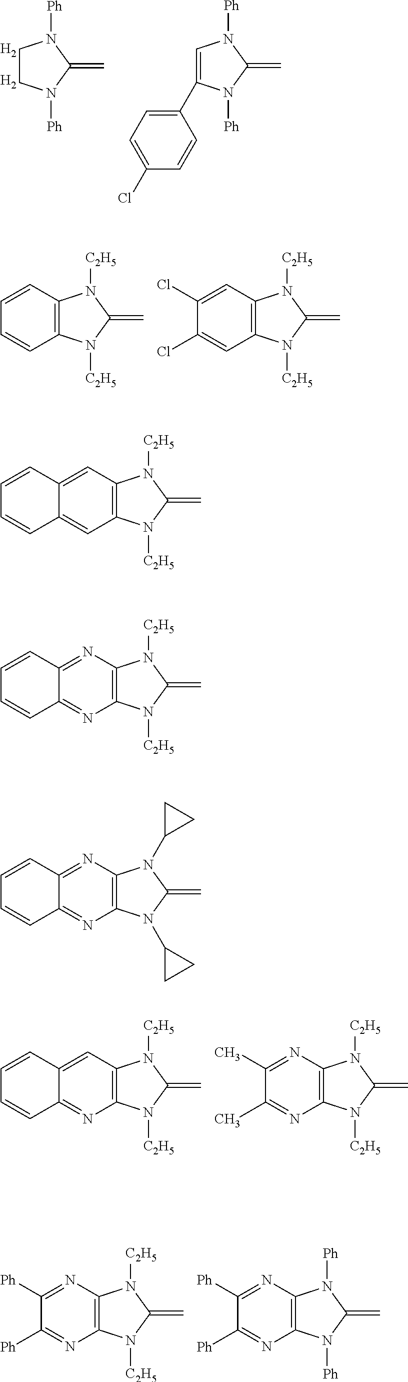

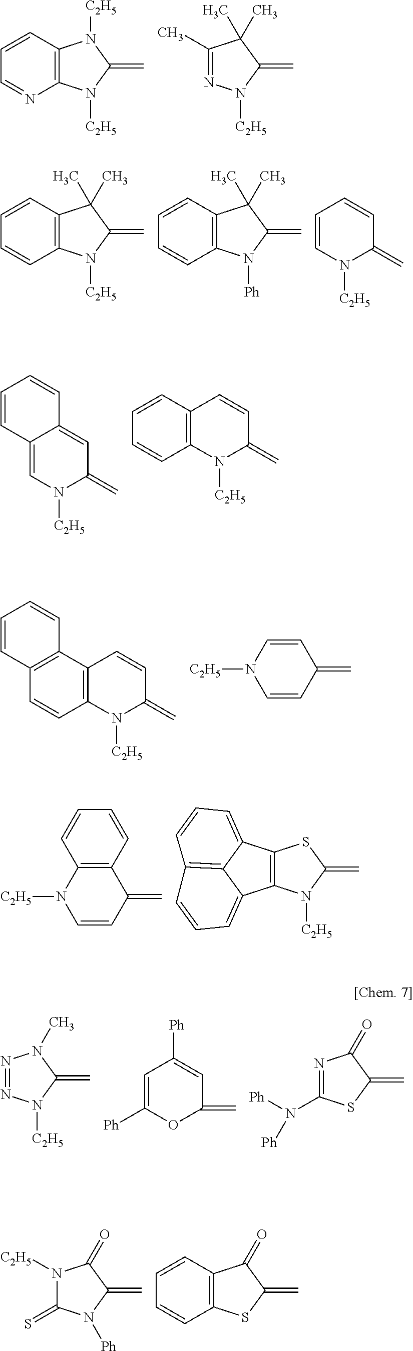

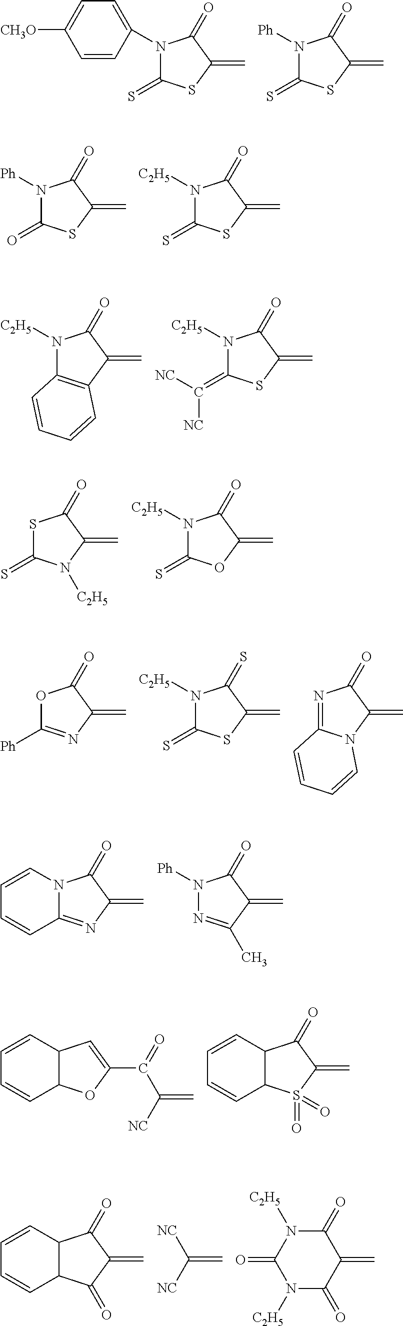

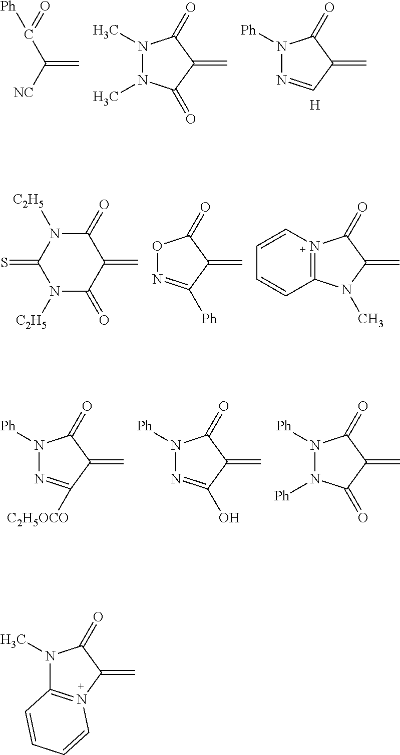

Ph in the above groups represents a phenyl group.

In Formula (1), each of L₁, L₂, and L₃ independently represents a substituted or unsubstituted methine group. Moreover, substituted methine groups may form a ring by being bonded to each other. The type of ring to be formed is not particularly limited, and examples thereof include the ring R which will be described later. Examples of substituents of the substituted methine group include the substituent W which will be described later. However, it is preferable for all of L₁, L₂, and L₃ to be an unsubstituted methine group.

Ar₁ and L₁ may form a ring by being bonded to each other. The type of ring to be formed in not particularly limited, and examples thereof include the ring R which will be described later. Moreover, the ring have further have a substituent, and examples of the substituent include the substituent W which will be described later. As the substituent, an alkyl group, a phenyl group, or the like is preferable.

In Formula (1), n represents an integer of 0 or greater, preferably represents an integer of 0 to 3, more preferably represents 0. If n is increased, the absorption wavelength region can be made to absorb light of a long wavelength, but a pyrolysis temperature is lowered. In view of appropriately absorbing light in a visible region and suppressing pyrolysis at the time of forming a film by vapor deposition, n is preferably 0.

The arylene group represented by Ar₁ is preferably an arylene group having 6 to 30 carbon atoms, and more preferably an arylene group having 6 to 18 carbon atoms. The arylene group may have the substituent W which will be described later, and is preferably an arylene group having 6 to 18 carbon atoms that may have a halogen atom or an alkyl group having 1 to 4 carbon atoms. Preferable examples of the arylene group include a phenylene group, a naphthylene group, an anthracenyl group, a pyrenylene group, a phenanthrenylene group, a methylphenylene group, a dimethylphenylene group, and the like. Among these, a phenylene group or a naphthylene group is more preferable.

The heteroarylene group represented by Ar₁ preferably has 3 to 30 carbon atoms, and more preferably has 4 to 18 carbon atoms. The heteroarylene group may have the substituent W which will be described later, and is preferably an arylene group having 4 to 18 carbon atoms that may have an alkyl group having 1 to 4 carbon atoms. Preferable examples of the heteroarylene structure include thiophene, furan, pyrrole, oxazole, diazole, thiazole, and benzo-condensed ring derivatives and thieno-condensed ring derivative of these. Among these, thiophene, benzothiophene, thienothiophene, dibenzothiophene, and bithienothiophene are more preferable.

Moreover, in view of superiority in various characteristics such as efficiency, response, and sensitivity of the photoelectric conversion element, Ar₁ is preferably a substituted or unsubstituted arylene group.

Each of Ar₂ and Ar₃ preferably independently represents an aryl group having 6 to 30 carbon atoms and more preferably independently represents an aryl group having 6 to 18 carbon atoms. The aryl group may have a substituent and is preferably an aryl group having 6 to 18 carbon atoms that may have a halogen atom, a halogenated alkyl group, an alkyl group having 1 to 4 carbon atoms, or an aryl group having 6 to 18 carbon atoms. The aryl group is more preferably an aryl group having 6 to 18 carbon atoms that has a halogen atom. Preferable examples of the aryl group include a phenyl group, a naphthyl group, an antrhacenyl group, a pyrenyl group, a phenanthrenyl group, a methylphenyl group, a dimethylphenyl group, a biphenyl group, and the like. Among these, a phenyl group, a naphthyl group, and a biphenyl group are more preferable.

Each of Ar₂ and Ar₃ preferably independently represents a heterocyclic group (heteroaryl group) having 3 to 30 carbon atoms, and more preferably independently represents a heteroaryl group having 3 to 18 carbon atoms. The heterocyclic group may have a substituent, and is preferably a heterocyclic group having 3 to 18 carbon atoms that may have a halogen atom, a halogenated alkyl group, an alkyl group having 1 to 4 carbon atoms, or an aryl group having 6 to 18 carbon atoms. The heterocyclic group preferably has a condensed ring structure, and preferably has a condensed ring structure consisting of a combination of rings (these rings may be the same as each other) selected from a furan ring, a thiophene ring, a selenophene ring, a silole ring, a pyridine ring, a pyrazine ring, a pyrimidine ring, an oxazole ring, a triazole ring, a triazole ring, an oxadiazole ring, and a thiadiazole ring. The heterocycle is more preferably a quinoline ring, an isoquinoline ring, a benzothiophene ring, a dibenzothiophene ring, a thienothiophene ring, a bithienobenzene ring, or a bithienothiophene ring.

In Formula (1), Ar₁ and Ar₂, Ar₁ and Ar₃, and Ar₂ and Ar₃ may form a ring by being bonded to each other respectively, and at least either Ar₁ and Ar₂ or Ar₁ and Ar₃ forms a ring by being bonded to each other. When these groups are bonded to each other, it is preferable for Ar₁ and Ar₂, Ar₁ and Ar₃, or Ar₂ and Ar₃ to form a ring by being directly bonded to each other respectively or by being bonded to each other through a linking group. If such a ring structure is formed, heat resistance of the compound represented by Formula (1) is improved, a photoelectric conversion element can be produced at a high vapor deposition rate under a high-temperature condition, and various performances such as sensitivity, response, and efficiency can be improved.

The structure of the linking group is not particularly limited, and examples of the linking group include an oxygen atom, a sulfur atom, an alkylene group, a silylene group, an alkenylene group, a cycloalkylene group, a cycloalkenylene group, an arylene group, a divalent heterocyclic group, an imino group, a substituted or unsubstituted nitrogen atom, and a group as a combination of these. These may further have a substituent, and examples of the substituent include an alkyl group which may have a substituent, an aryl group which may have a substituent, and the like. The linking group is preferably an oxygen atom, an alkyl group, a silylene group, an alkenylene group, a cycloalkylene group, a cycloalkenylene group, or an arylene group, and more preferably an oxygen atom or an alkylene group.

Moreover, either Ar₁ and Ar₂ or Ar₁ and Ar₃ may form a ring, or alternatively, Ar₁ and Ar₂ as well as Ar₁ and Ar₃ may form a ring. Furthermore, Ar₁ and Ar₂ as well as Ar₂ and Ar₃ may form a ring, Ar₁ and Ar₃ as well as Ar₂ and Ar₃ may form a ring, and all of Ar₁ and Ar₂, Ar₁ and Ar₃, and Ar₂ and Ar₃ may form a ring.

A substituent may be introduced into Ar₁, Ar₂, and Ar₃ as described above, and the introduction position is not particularly limited. However, in view of higher melting point and better vapor deposition characteristics, the substituent is preferably introduced in a para-position with respect to a position where Ar₁, Ar₂, and Ar₃ are substituted with (bonded to) a nitrogen atom.

In the compound represented by Formula (1), at least one of Z₁, Ar₁, Ar₂, and Ar₃ is substituted with a halogen group or a halogenated alkyl group. As described above, if the group is substituted with a halogen group or a halogenated alkyl group, interaction between molecules of the compound represented by Formula (1) is weakened; sublimation properties are improved; a degree of freedom of the compound is reduced; a melting point is heightened; and as a result, vapor deposition characteristics (heat resistance) are improved. Particularly, it is preferable for the group to be substituted with a halogen atom, since various characteristics of the photoelectric conversion element are further improved.

The type of halogen group is not particularly limited, and examples thereof include a fluorine group (—F), a chloro group (—Cl), a bromine group (Br—), an iodine group (I—), and the like. Among these, a fluorine group and a chloro group are more preferable, since vapor deposition characteristics and various characteristics of the photoelectric conversion element are further improved. Particularly, the type of halogen group of the halogen group or the halogenated alkyl group that Z₁ has is preferably a chloro group, and the type of halogen group of the halogen group or the halogenated alkyl group that Ar₁, Ar₂, and Ar₃ have is preferably a fluorine group.

The type of halogen group of the halogenated alkyl group is as described above, and preferable embodiments thereof are also the same. The number of carbon atom contained in the halogenated alkyl group is not particularly limited, but is preferably 1 to 3, and more preferably 1. The number of halogen atom contained in the halogenated alkyl group is not particularly limited. However, in view of facilitating synthesis, the number of halogen atom is preferably 1 to 5.

The number (total number) of halogen group and halogenated alkyl group with which at least one of Z₁, Ar₁, Ar₂, and Ar₃ is substituted is not particularly limited. However, the number is preferably 1 to 4, and more preferably 1 to 2, since various characteristics of the photoelectric conversion element are further improved.

When Z₁ is the group represented by Formula (Z2) described above, and a halogen group or a halogenated alkyl group is introduced into the group, at least one of R₁₁ to R₁₄ is a halogen group or a halogenated alkyl group. Alternatively, at least one of R₁₁ to R₁₄ is a group having a substituent (for example, an alkyl group having a substituent or an aryl group having a substituent), and the substituent is a halogen group or a halogenated alkyl group. Particularly, in view of better vapor deposition characteristics, it is preferable for a halogen group to be introduced into R₁₂ and R₁₃.

Moreover, when Z₁ is the group represented by Formula (Z3) described above, and a halogen group or a halogenated alkyl group is introduced into the group, at least one of R₂₁ to R₂₆ is a halogen group or a halogenated alkyl group. Alternatively, at least one of R₂₁ to R₂₆ is a group having a substituent (for example, an alkyl group having a substituent or an aryl group having a substituent), and the substituent is a halogen group or a halogenated alkyl group. Particularly, in view of better vapor deposition characteristics, it is preferable for a halogen atom to be introduced into R₂₃ and R₂₄.

Furthermore, when Z₁ is a group represented by Formula (Z4) described above, and a halogen group or a halogenated alkyl group is introduced into the group, at least one of R₃₁ and R₃₂ is a halogen group or a halogenated alkyl group. Alternatively, at least one of R₃₁ and R₃₂ is a group having a substituent (for example, an alkyl group having a substituent or an aryl group having a substituent), and the substituent is a halogen group or a halogenated alkyl group. Particularly, in view of better vapor deposition characteristics, it is preferable for a halogen atom to be introduced into R₃₁ and R₃₂.

The substituent W in the present specification will be described below.

Examples of the substituent W include halogen atoms, alkyl groups (including cycloalkyl groups, bicycloalkyl groups, and tricycloalkyl groups), alkenyl groups (including cycloalkenlyl groups and bicycloalkenyl groups), alkynyl groups, aryl groups, heterocyclic groups, cyano groups, hydroxy groups, nitro groups, carboxy groups, alkoxy groups, aryloxy groups, silyloxy groups, heterocyclic oxy groups, acyloxy groups, carbamoyloxy groups, alkoxycarbonyloxy groups, aryloxycarbonyloxy groups, amino groups (including anilino groups), ammonio groups, acylamino groups, aminocarbonylamino groups, alkoxycarbonylamino groups, aryloxycarbonylamino groups, sulfamoylamino groups, alkyl or aryl sulfonylamino groups, mercapto groups, alkylthio groups, arylthio groups, heterocyclic thio groups, sulfamoyl groups, sulfo groups, alkyl or aryl sulfinyl groups, alkyl or aryl sulfonyl groups, acyl groups, aryloxycarbonyl groups, alkoxycarbonyl groups, carbamoyl groups, aryl or heterocyclic azo groups, imide groups, phosphino groups, phosphinyl groups, phosphinyloxy groups, phosphinylamino groups, phosphono groups, silyl groups, hydrazino groups, ureido groups, boronic acid groups (—B(OH)₂), phosphato groups (—OPO(OH)₂), sulphato groups (—OSO₃H), and other known substituents.

The substituent W is described in detail in paragraph [0023] of JP 2007-234651 A.

Moreover, two substituents W may form a ring in combination with each other. Examples of such a ring include aromatic or non-aromatic hydrocarbon ring, an aromatic or non-aromatic heterocycle, and a polycyclic condensed ring formed when the above rings are combined with each other. Specifically, examples of the ring include the specific examples of the ring R which will be described later.

Among the substituents W described above, the substituents having a hydrogen atom may be further substituted with the aforementioned group after the hydrogen atom is removed.

[Ring R]

In the present specification, examples of the ring R include aromatic or non-aromatic hydrocarbon rings or heterocycles and polycyclic condensed rings which are formed when the above rings are combined with each other. Examples thereof include a benzene ring, a naphthalene ring, an anthracene ring, a phenanthrene ring, a fluorene ring, a triphenylene ring, a naphthacene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an indolidine ring, an indole ring, a benzofuran ring, a benzothiophene ring, an isobenzofuran ring, a quinolizine ring, a quinoline ring, a phthalazine ring, a naphthyridine ring, a quinoxaline ring, a quinoxazoline ring, an isoquinoline ring, a carbazole ring, a phenanthridine ring, an acridine ring, a phenanthroline ring, a thianthrene ring, a chromene ring, a xanthene ring, a phenoxathiin ring, a phenothiazine ring, and a phenazine ring.

It is preferable for the compound represented by Formula (1) to have an absorption maximum at a wavelength equal to or longer than 400 nm or but shorter than 720 nm in an ultraviolet and visible absorption spectrum. From the viewpoint of absorbing light over a wide range of visible region, a peak wavelength (absorption maximum wavelength) of the absorption spectrum is preferably from 450 nm to 700 nm, more preferably from 480 nm to 700 nm, and particularly preferably from 510 nm to 680 nm.

The absorption maximum wavelength of the compound can be measured by measuring a chloroform solution of the compound by using UV-2550 manufactured by Shimadzu Corporation. A concentration of the chloroform solution is preferably 5×10⁻⁵ mol/l to 1×10⁻⁷ mol/l, more preferably 3×10⁻⁵ mol/l to 2×10⁻⁶ mol/l, and particularly preferably 2×10⁻⁵ mol/l to 5×10⁻⁶ mol/l.

It is preferable for the compound represented by Formula (1) to have an absorption maximum at a wavelength equal to or longer than 400 nm but shorter than 720 nm in an ultraviolet and visible absorption spectrum. Moreover, a molar absorption coefficient of the absorption maximum wavelength is preferably equal to or greater than 10,000 mol⁻¹·l·cm⁻¹. In order to reduce the film thickness of the photoelectric conversion layer and to obtain an element having a high charge-collecting efficiency and high sensitivity characteristics, materials having a great molar absorption coefficient are preferable. The molar absorption coefficient of the compound represented by Formula (1) is more preferably equal to or greater than 30,000 mol⁻¹·l·cm⁻¹, even more preferably equal to or greater than 50,000 mol⁻¹·l·cm⁻¹, particularly preferably equal to or greater than 60,000 mol⁻¹·l·cm⁻¹, and most preferably equal to or greater than 70,000 mol⁻¹·l·cm⁻¹. The molar absorption coefficient of the compound represented by Formula (1) is measured using the chloroform solution.

The greater the difference between a melting point of the compound represented by Formula (1) and a vapor deposition temperature thereof (melting point−vapor deposition temperature difference) becomes, the more difficult it is for the compound to be degraded at the time of vapor deposition and the more possible it is to increase the vapor deposition temperature in order to heighten the vapor deposition speed. The difference between the melting point and the vapor deposition temperature (melting point−vapor deposition temperature difference) is preferably equal to or higher than 60° C., more preferably equal to or higher than 80° C., even more preferably equal to or higher than 90° C., and particularly preferably equal to or higher than 100° C.

Furthermore, the melting point of the compound represented by Formula (1) is preferably equal to or higher than 240° C., more preferably equal to or higher than 280° C., and even more preferably equal to or higher than 300° C. If the melting point is 300° C. or higher, it is preferable since the compound is less likely to be melted before vapor deposition and can be stably formed into a film, and besides, a degradation product of the compound is not easily generated and accordingly, photoelectric conversion performance does not easily deteriorate.

The vapor deposition temperature of the compound is a temperature at a point time when the vapor deposition speed becomes 0.4 Å/s (0.4×10⁻¹⁰ m/s) by heating the compound at a degree of vacuum of equal to or lower than 4×10⁻⁴ Pa.

A molecular weight of the compound represented by Formula (1) is preferably 300 to 1,500, more preferably 500 to 1,000, and particularly preferably 500 to 900. If the molecular weight of the compound is equal to or smaller than 1,500, the vapor deposition temperature is not increased, and the compound is not easily degraded. If the molecular weight of the compound is equal to or greater than 300, a glass transition point of a vapor-deposited film is not lowered, and heat resistance of the element does not easily deteriorate.

A glass transition point (Tg) of the compound represented by Formula (1) is preferably equal to or higher than 95° C., more preferably equal to or higher than 110° C., even more preferably equal to or higher than 135° C., particularly preferably equal to or higher than 150° C., and most preferably equal to or higher than 160° C. The higher the glass transition point, the better, since heat resistance of the element is improved.

The compound represented by Formula (1) is useful particularly as a material of a photoelectric conversion film used for an imaging device, a photosensor, or a solar cell. Generally, the compound represented by Formula (1) functions as a p-type organic compound in the photoelectric conversion film. Moreover, the compound can also be used as a coloring material, a liquid crystal material, a material of an organic semiconductor, a material of an organic light-emitting element, a charge-transporting material, a pharmaceutical material, a material of a fluorescent diagnostic agent, and the like.

### Preferable Embodiments

Examples of preferable embodiments of the compound represented by Formula (1) include compounds represented by the following Formulae (2) to (5). These compounds are superior in terms of the vapor deposition characteristics of the compound and/or various characteristics of the photoelectric conversion element. Particularly, a compound represented by Formula (2) is preferable since the effects of the present invention are further improved.

Hereinafter, the embodiments of the compounds represented by Formulae (2) to (5) will be described in detail.

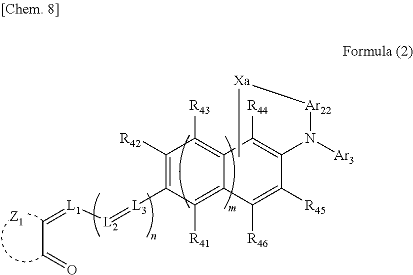

In Formula (2), Z₁, L₁, L₂, L₃, Ar₃, and n have the same definition as the respective groups in Formula (1), and preferable embodiments thereof are also the same.

Each of R₄₁ to R₄₆ independently represents a hydrogen atom or a substituent. Each of R₄₂ and R₄₃, R₄₃ and R₄₄, R₄₅ and R₄₆, and R₄₁ and R₄₆ may independently form a ring.

Moreover, Ar₂₂ and Ar₃ as well as Ar₃ and R₄₁ to R₄₆ may form a ring by being bonded to each other respectively.

Ar₂₂ represents a substituted or unsubstituted divalent arylene group or a substituted or unsubstituted divalent heteroarylene group. The definition and preferable embodiments of the respective groups represented by Ar₂₂ are the same as the definition and preferable embodiments described for Ar₁.

Examples of the substituent represented by R₄₁ to R₄₆ include the substituent W described above. As the substituent, a hydrogen atom, a halogen atom, an alkyl group having 1 to 18 carbon atoms, an aryl group having 6 to 18 carbon atoms, and a heterocyclic group having 4 to 16 carbon atoms are preferable; a hydrogen atom, an alkyl group having 1 to 12 carbon atoms, an aryl group having 6 to 14 carbon atoms, and a fluorine atom are more preferable; and a hydrogen atom, an alkyl group having 1 to 6 carbon atoms, and an aryl group having 6 to 10 are even more preferable. Among these, a hydrogen atom, a fluorine atom, a methyl group, an ethyl group, a propyl group, a butyl group, a hexyl group, a cyclohexyl group, a phenyl group, and a naphthyl group are preferable, and a hydrogen atom, a fluorine atom, a methyl group, a butyl group, a hexyl group, and a phenyl group are particularly preferable. The alkyl group may be branched.

These may further have a substituent. Specific examples of the substituent include the substituent W described above. As the substituent, a halogen atom, an alkyl group, an aryl group, a heterocyclic group, a hydroxyl group, an amino group, and a mercapto group are preferable; a halogen atom, an alkyl group, an aryl group, and a heterocyclic group are more preferable; a fluorine atom, an alkyl group, and an aryl group are even more preferable; an alkyl group and an aryl group are still more preferable; and a alkyl group is most preferable.

Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent. Xa is linked to Ar₂₂ and also forms a link as one of the groups R₄₁ to R₄₆. In other words, the “Xa forms a link as one of the groups R₄₁ to R₄₆” means that Xa is linked to a carbon atom on a benzene ring instead of one of the groups R₄₁ to R₄₆ to be linked to the carbon atom. Among the above, in view of superiority in vapor deposition characteristics of the compound and/or superiority in various characteristics of the photoelectric conversion element, an alkylene group, a silylene group, an alkenylene group, a cycloalkylene group, a cycloalkenylene group, and an arylene group are preferable; an alkylene group, an alkenylene group, and a cycloalkenylene group are more preferable; and an alkylene group is even more preferable. When Xa further has a substituent, examples of the substituent include the substituent W described above. The substituent is preferably a hydrogen atom, a halogen atom, an alkyl group having 1 to 18 carbon atoms, an aryl group having 6 to 18 carbon atoms, or a heterocyclic group having 4 to 16 carbon atoms, more preferably a hydrogen atom, an alkyl group having 1 to 12 carbon atoms, an aryl group having 6 to 14 carbon atoms, or a fluorine atom, and even more preferably a hydrogen atom, an alkyl group having 1 to 6 carbon atoms, or an aryl group having 6 to 10 carbon atoms.

m represents 0 or 1. Particularly, in view of superiority in vapor deposition characteristics of the compound and/or superiority in various characteristics of the photoelectric conversion element, m is preferably 1.

In Formula (2), at least one of Z₁, Ar₂₂, and Ar₃ is substituted with a halogen group or a halogenated alkyl group, or alternatively, at least one of R₄₁ to R₄₆ is a halogen group or a halogenated alkyl group. As another option, Xa has substituents, and at least one of the substituents is a halogen group or a halogenated alkyl group.

One of the preferable embodiments of Formula (2) is the following Formula (2-a).

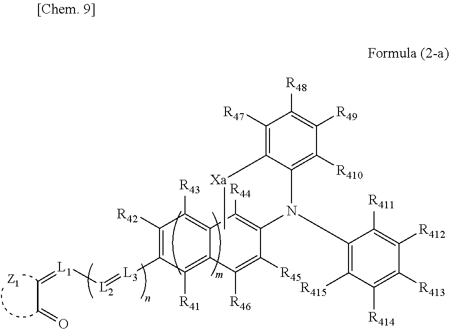

In Formula (2-a), Z₁, L₁, L₂, L₃, Xa, R₄₁ to R₄₆, m, and n have the same definition as the respective groups in Formula (2), and preferable embodiments thereof are also the same.

Each of R₄₇ to R₄₉, R₄₁₀, and R₄₁₁ to R₄₁₅ independently represents a hydrogen atom or a substituent. Examples of the substituent include the substituent W described above. The substituent is preferably a hydrogen atom, a halogen atom, an alkyl group having 1 to 18 carbon atoms, an aryl group having 6 to 18 carbon atoms, or a heterocyclic group having 4 to 16 carbon atoms, more preferably a hydrogen atom, an alkyl group having 1 to 12 carbon atoms, an aryl group having 6 to 14 carbon atoms, or a fluorine atom, and even more preferably a hydrogen atom, an alkyl group having 1 to 6 carbon atoms, or an aryl group having 6 to 10 carbon atoms. Among these, a hydrogen atom, a fluorine atom, a methyl group, an ethyl group, a propyl group, a butyl group, a hexyl wlq, a cyclohexyl group, a phenyl group, and a naphthyl group are preferable, and a hydrogen atom, a fluorine atom, a methyl group, and a phenyl group are particularly preferable. The alkyl group may be branched.

These may further have a substituent. Specific examples of the substituent include the substituent W described above. The substituent is preferably a halogen atom, an alkyl group, an aryl group, a heterocyclic group, a hydroxyl group, an amino group, or a mercapto group, more preferably a halogen atom, an alkyl group, an aryl group, or a heterocyclic group, even more preferably a fluorine atom, an alkyl group, or an aryl group, and particularly preferably a fluorine atom, a methyl group, a phenyl group, or a 4-fluorophenyl group.

Among R₄₇ to R₄₉, R₄₁₀, and R₄₁₁ to R₄₁₅, groups adjacent to each other may form a ring by being bonded to each other. Examples of the ring to be formed include the ring R described above. The ring to be formed is preferably an aromatic or non-aromatic heterocycle. Specific examples thereof include a benzene ring, a naphthalene ring, an anthracene ring, a pyridine ring, a pyrimidine ring, a fluorene ring (configured with benzene rings to which R₄₇ to R₄₉, R₄₁₀, and R₄₁₁ to R₄₁₅ are bonded), and the like.

Moreover, R₄₁₀ and R₄₁₁ as well as R₄₁₅ and R₄₅ may be linked to each other respectively. When these are linked to each other, a preferable range of the linking group is the same as the preferable range of Xa of Formula (2). Particularly, it is preferable for them to be linked to each other through an alkylene group. When R₄₁₀ and R₄₁₁ as well as R₄₁₅ and R₄₅ are linked to each other, they may form a 5- to 10-membered ring (preferably a 5- to 6-membered ring, and more preferably a 6-membered ring) together with a N atom. Furthermore, R₄₁₀ and R₄₁₁ as well as R₄₁₅ and R₄₅ may be linked to each other through a single bond.

When m=0, Xa preferably forms a link as R₄₄ or R₄₅. In other words, it is preferable that Xa be linked to a carbon atom on a benzene ring instead of R₄₄ or R₄₅ to be linked to the carbon atom. When m=1, Xa preferably forms a link as R₄₄ or R₄₅. More preferably, Xa forms a link as R₄₄.

In Formula (2-a), Z₁ is substituted with a halogen group or a halogenated alkyl group, or alternatively, at least one of R₄₁ to R₄₆, R₄₇ to R₄₉, R₄₁₀, and R₄₁₁ to R₄₁₅ is a halogen atom or a halogenated alkyl group. As another option, Xa has substituents, and at least one of the substituents is a halogen group or a halogenated alkyl group.

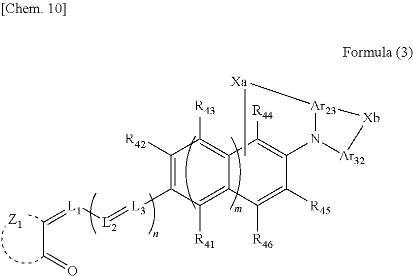

In Formula (3), Z₁, L₁, L₂, L₃, R₄₁ to R₄₆, Xa, m, and n have the same definition as the respective groups in Formula (2), and preferable embodiments thereof are also the same.

Ar₂₃ represents a substituted or unsubstituted trivalent aromatic hydrocarbon group (an arene group) or a substituted or unsubstituted trivalent aromatic heterocyclic group (a heterocyclic group or a heteroarene group).

The aromatic hydrocarbon group preferably has 6 to 30 carbon atoms, and more preferably has 6 to 18 carbon atoms. The aromatic hydrocarbon group may have a substituent, and preferably is a trivalent aromatic hydrocarbon group having 6 to 18 carbon atoms that may have a alkyl group having 1 to 4 carbon atoms or an aryl group having 6 to 18 carbon atoms.

The aromatic heterocyclic group preferably has 3 to 30 carbon atoms, and more preferably has 3 to 18 carbon atoms. The heterocyclic group may have a substituent, and preferably is an aromatic heterocyclic group having 3 to 18 carbon atoms that may have an alkyl group having 1 to 4 carbon atoms or an aryl group having 6 to 18 carbon atoms.

Ar₃₂ represents a substituted or unsubstituted divalent arylene group or a substituted or unsubstituted divalent heteroarylene group. The definition and preferable embodiments of the respective groups represented by Ar₃₂ are the same as the definition and preferable embodiments described for Ar₁.

Xb represents a single bond, an oxygen atom, a sulfur atom, an alkylene group, a silylene group, an alkenylene group, a cycloalkylene group, a cycloalkenylene group, an arylene group, a divalent heterocyclic group, or an imino group. These may further have a substituent and are linked to Ar₂₃ and Ar₃₂. Among these, in view of superiority in various characteristics of the photoelectric conversion element, an alkylene group, a silylene group, an alkenylene group, a cycloalkylene group, a cycloalkenylene group, and an arylene group are preferable.

In Formula (3), at least one of Z₁, Ar₂₃, and Ar₃₂ is substituted with a halogen group or a halogenated alkyl group, or alternatively, at least one of R₄₁ to R₄₆ is a halogen group or a halogenated alkyl group. As another option, Xa and/or Xb have substituents, and at least one of the substituents is a halogen group or a halogenated alkyl group.

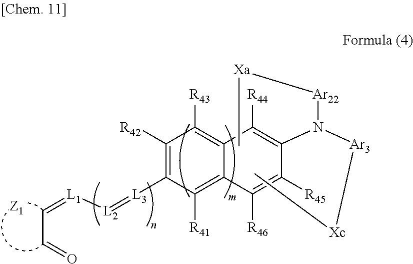

In Formula (4), Z₁, L₁, L₂, L₃, R₄₁ to R₄₆, Xa, Ar₂₂, Ar₃₂, m, and n have the same definition as the respective groups in Formula (2), and preferable embodiments thereof are also the same.

Xc represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent. Xc is linked to Ar₃₂ and also forms a link as one of the groups R₄₁ to R₄₆. As Xc, in view of superiority in various characteristics of the photoelectric conversion element, an alkylene group, a silylene group, an alkenylene group, a cycloalkylene group, a cycloalkenylene group, and an arylene group are preferable.

In Formula (4), at least one of Z₁, Ar₂₂, and Ar₃₂ is substituted with a halogen group or a halogenated alkyl group, or alternatively, at least one of R₄₁ to R₄₆ is a halogen group or a halogenated alkyl group. As another option, Xa and/or Xc have substituents, and at least one of the substituents is a halogen group or a halogenated alkyl group.

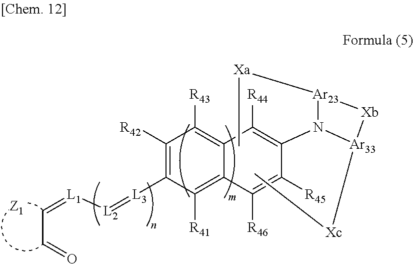

In Formula (5), Z₁, L₁, L₂, L₃, R₄₁ to R₄₆, Xa, Ar₂₃, m, and n have the same definition as the respective groups in Formula (2), and preferable embodiments thereof are also the same.

In Formula (5), Xb has the same definition as Xb in Formula (3), and preferable embodiments thereof are also the same.

In Formula (5), Xc has the same definition as Xc in Formula (4), and preferable embodiments thereof are also the same.

Ar₃₃ represents a substituted or unsubstituted trivalent aromatic hydrocarbon group (an arene group) or a substituted or unsubstituted aromatic heterocyclic group (a heterocyclic group or a heteroarene group). The definition and preferable embodiments of the respective groups represented by Ar₃₃ are the same as the definition and preferable embodiments described for Ar₂₃.

In Formula (5), at least one of Z₁, Ar₂₃, and Ar₃₃ is substituted with a halogen group or a halogenated alkyl group, or alternatively, at least one of R₄₁ to R₄₆ is a halogen group or a halogenated alkyl group. As another option, at least one of Xa, Xb, and Xc has substituents, and at least one of the substituents is a halogen group or a halogenated alkyl group.

The compound represented by Formula (1) can be produced based on the synthesis method described in, for example, JP 2000-297068 A. Specific examples of the compound represented by Formula (1) will be shown below, but the present invention is not limited thereto.

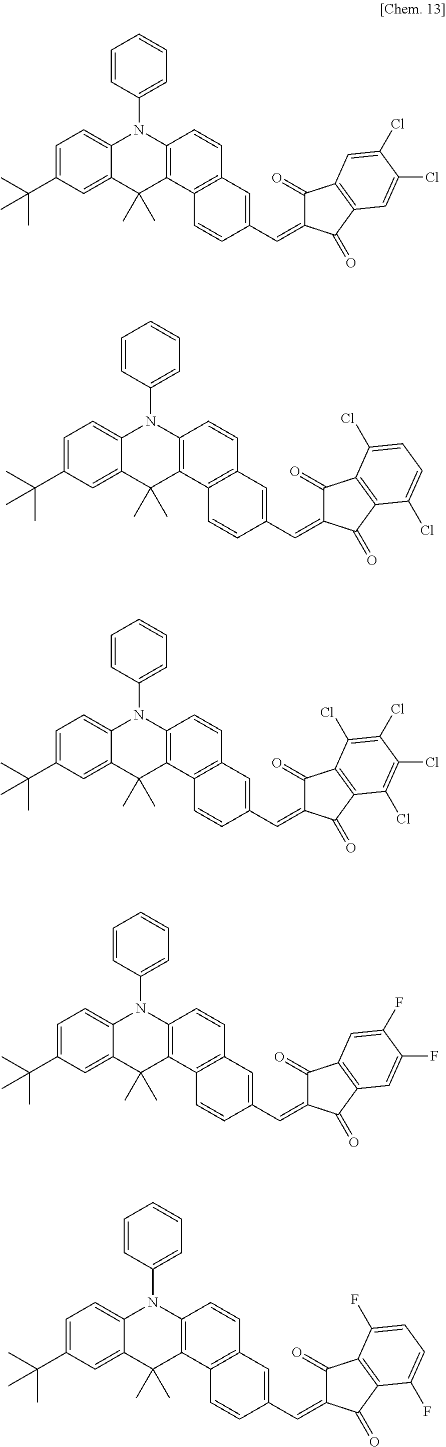 [SMILES: CC(C)(C)c1ccc2c(c1)C(C)(C)c1c(ccc3cc(C=C4C(=O)c5cc(Cl)c(Cl)cc5C4=O)ccc13)N2c1ccccc1 ; CC(C)(C)c1ccc2c(c1)C(C)(C)c1c(ccc3cc(C=C4C(=O)c5c(Cl)ccc(Cl)c5C4=O)ccc13)N2c1ccccc1 ; CC(C)(C)c1ccc2c(c1)C(C)(C)c1c(ccc3cc(C=C4C(=O)c5c(Cl)c(Cl)c(Cl)c(Cl)c5C4=O)ccc13)N2c1ccccc1 ; CC(C)(C)c1ccc2c(c1)C(C)(C)c1c(ccc3cc(C=C4C(=O)c5cc(F)c(F)cc5C4=O)ccc13)N2c1ccccc1 ; CC(C)(C)c1ccc2c(c1)C(C)(C)c1c(ccc3cc(C=C4C(=O)c5c(F)ccc(F)c5C4=O)ccc13)N2c1ccccc1]

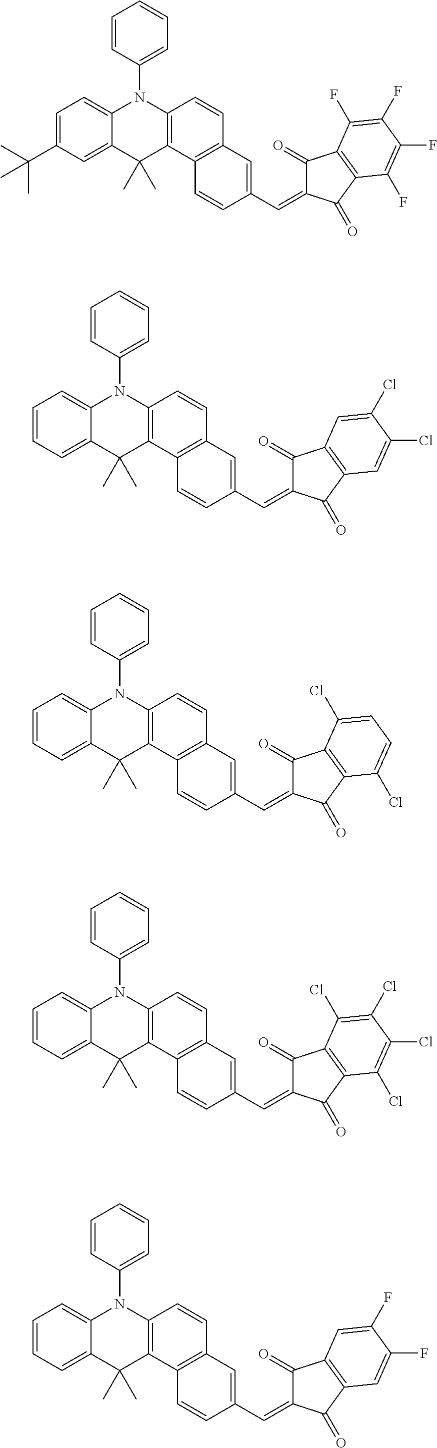 [SMILES: CC(C)(C)c1ccc2c(c1)C(C)(C)c1c(ccc3cc(C=C4C(=O)c5c(F)c(F)c(F)c(F)c5C4=O)ccc13)N2c1ccccc1 ; CC1(C)c2ccccc2N(c2ccccc2)c2ccc3cc(C=C4C(=O)c5cc(Cl)c(Cl)cc5C4=O)ccc3c21 ; CC1(C)c2ccccc2N(c2ccccc2)c2ccc3cc(C=C4C(=O)c5c(Cl)ccc(Cl)c5C4=O)ccc3c21 ; CC1(C)c2ccccc2N(c2ccccc2)c2ccc3cc(C=C4C(=O)c5c(Cl)c(Cl)c(Cl)c(Cl)c5C4=O)ccc3c21 ; CC1(C)c2ccccc2N(c2ccccc2)c2ccc3cc(C=C4C(=O)c5cc(F)c(F)cc5C4=O)ccc3c21]

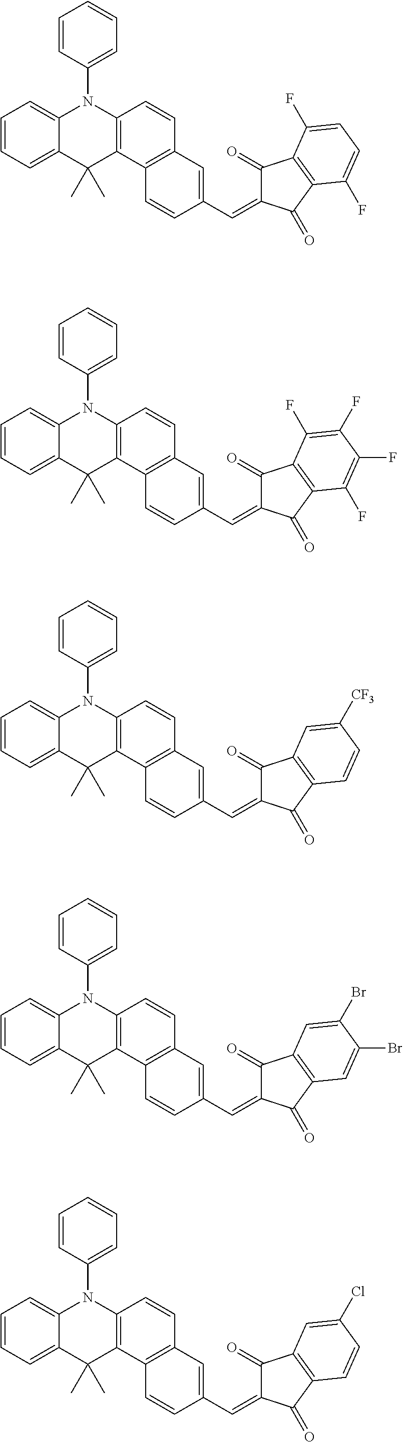 [SMILES: CC1(C)c2ccccc2N(c2ccccc2)c2ccc3cc(C=C4C(=O)c5c(F)ccc(F)c5C4=O)ccc3c21 ; CC1(C)c2ccccc2N(c2ccccc2)c2ccc3cc(C=C4C(=O)c5c(F)c(F)c(F)c(F)c5C4=O)ccc3c21 ; CC1(C)c2ccccc2N(c2ccccc2)c2ccc3cc(/C=C4/C(=O)c5ccc(C(F)(F)F)cc5C4=O)ccc3c21 ; CC1(C)c2ccccc2N(c2ccccc2)c2ccc3cc(C=C4C(=O)c5cc(Br)c(Br)cc5C4=O)ccc3c21 ; CC1(C)c2ccccc2N(c2ccccc2)c2ccc3cc(/C=C4/C(=O)c5ccc(Cl)cc5C4=O)ccc3c21]

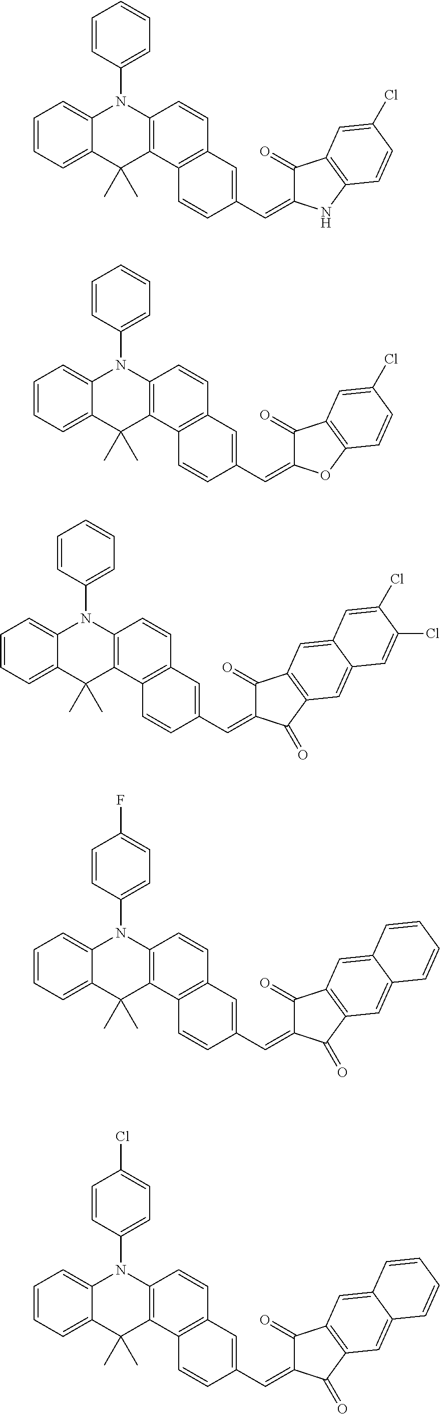 [SMILES: CC1(C)c2ccccc2N(c2ccccc2)c2ccc3cc(/C=C4/Nc5ccc(Cl)cc5C4=O)ccc3c21 ; CC1(C)c2ccccc2N(c2ccccc2)c2ccc3cc(/C=C4/Oc5ccc(Cl)cc5C4=O)ccc3c21 ; CC1(C)c2ccccc2N(c2ccccc2)c2ccc3cc(C=C4C(=O)c5cc6cc(Cl)c(Cl)cc6cc5C4=O)ccc3c21 ; CC1(C)c2ccccc2N(c2ccc(F)cc2)c2ccc3cc(C=C4C(=O)c5cc6ccccc6cc5C4=O)ccc3c21 ; CC1(C)c2ccccc2N(c2ccc(Cl)cc2)c2ccc3cc(C=C4C(=O)c5cc6ccccc6cc5C4=O)ccc3c21]

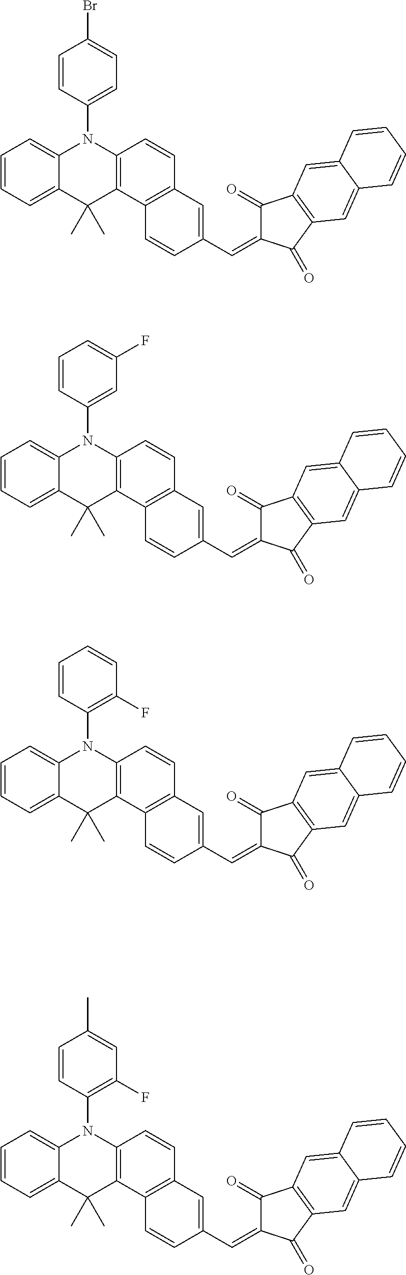 [SMILES: CC1(C)c2ccccc2N(c2ccc(Br)cc2)c2ccc3cc(C=C4C(=O)c5cc6ccccc6cc5C4=O)ccc3c21 ; CC1(C)c2ccccc2N(c2cccc(F)c2)c2ccc3cc(C=C4C(=O)c5cc6ccccc6cc5C4=O)ccc3c21 ; CC1(C)c2ccccc2N(c2ccccc2F)c2ccc3cc(C=C4C(=O)c5cc6ccccc6cc5C4=O)ccc3c21 ; Cc1ccc(N2c3ccccc3C(C)(C)c3c2ccc2cc(C=C4C(=O)c5cc6ccccc6cc5C4=O)ccc32)c(F)c1]

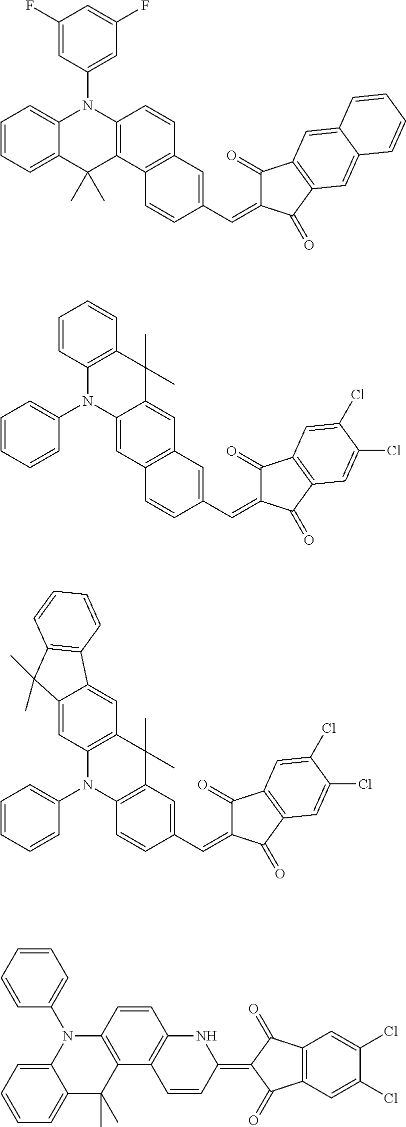 [SMILES: CC1(C)c2ccccc2N(c2cc(F)cc(F)c2)c2ccc3cc(C=C4C(=O)c5cc6ccccc6cc5C4=O)ccc3c21 ; CC1(C)c2ccccc2N(c2ccccc2)c2cc3ccc(C=C4C(=O)c5cc(Cl)c(Cl)cc5C4=O)cc3cc21 ; CC1(C)c2ccccc2-c2cc3c(cc21)N(c1ccccc1)c1ccc(C=C2C(=O)c4cc(Cl)c(Cl)cc4C2=O)cc1C3(C)C ; CC1(C)c2ccccc2N(c2ccccc2)c2ccc3c(c21)C=CC(=C1C(=O)c2cc(Cl)c(Cl)cc2C1=O)N3]

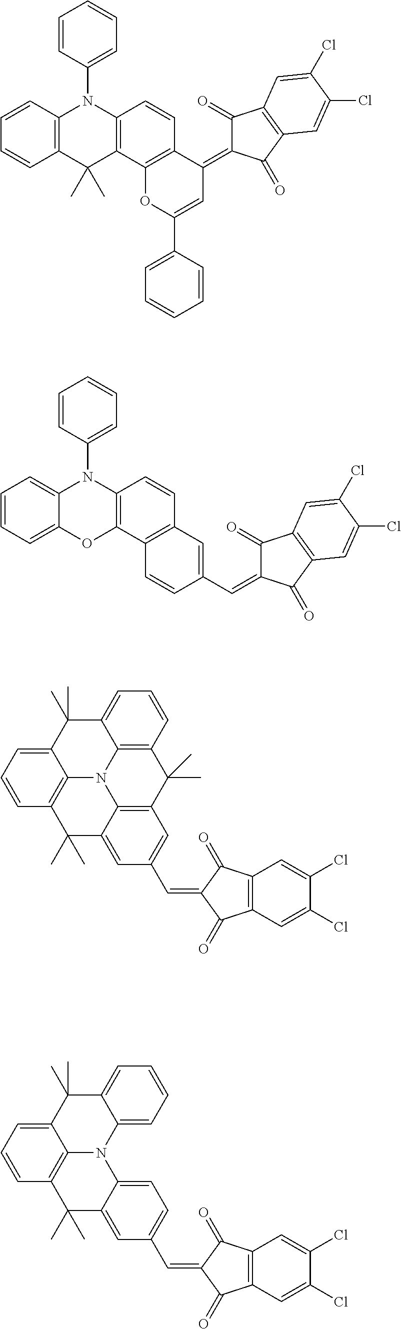 [SMILES: CC1(C)c2ccccc2N(c2ccccc2)c2ccc3c(c21)OC(c1ccccc1)=CC3=C1C(=O)c2cc(Cl)c(Cl)cc2C1=O ; O=C1C(=Cc2ccc3c4c(ccc3c2)N(c2ccccc2)c2ccccc2O4)C(=O)c2cc(Cl)c(Cl)cc21 ; CC1(C)c2cccc3c2N2c4c1cccc4C(C)(C)c1cc(C=C4C(=O)c5cc(Cl)c(Cl)cc5C4=O)cc(c12)C3(C)C ; CC1(C)c2ccccc2N2c3ccc(C=C4C(=O)c5cc(Cl)c(Cl)cc5C4=O)cc3C(C)(C)c3cccc1c32]

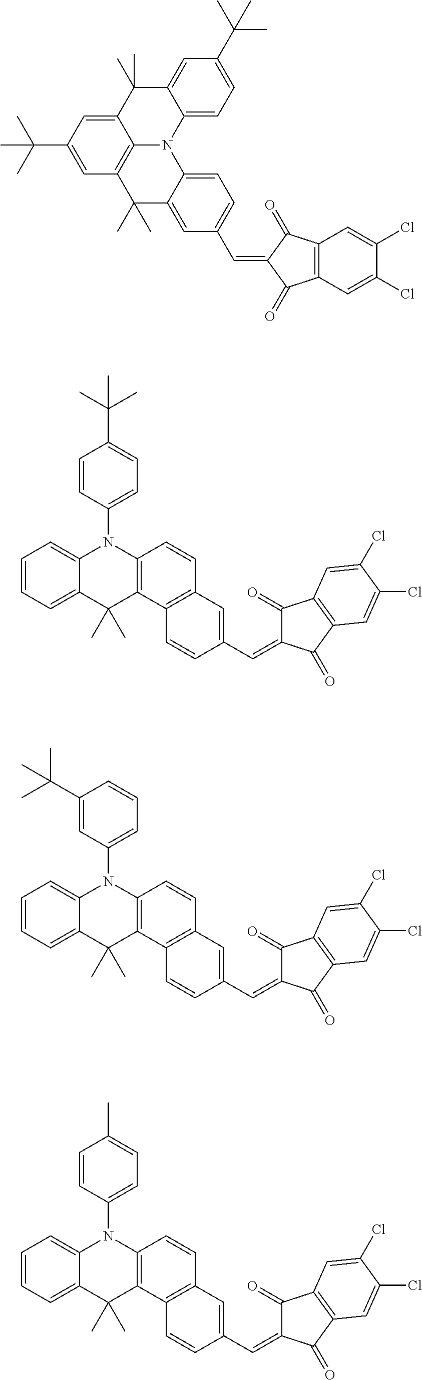 [SMILES: CC(C)(C)c1ccc2c(c1)C(C)(C)c1cc(C(C)(C)C)cc3c1N2c1ccc(C=C2C(=O)c4cc(Cl)c(Cl)cc4C2=O)cc1C3(C)C ; CC(C)(C)c1ccc(N2c3ccccc3C(C)(C)c3c2ccc2cc(C=C4C(=O)c5cc(Cl)c(Cl)cc5C4=O)ccc32)cc1 ; CC(C)(C)c1cccc(N2c3ccccc3C(C)(C)c3c2ccc2cc(C=C4C(=O)c5cc(Cl)c(Cl)cc5C4=O)ccc32)c1 ; Cc1ccc(N2c3ccccc3C(C)(C)c3c2ccc2cc(C=C4C(=O)c5cc(Cl)c(Cl)cc5C4=O)ccc32)cc1]

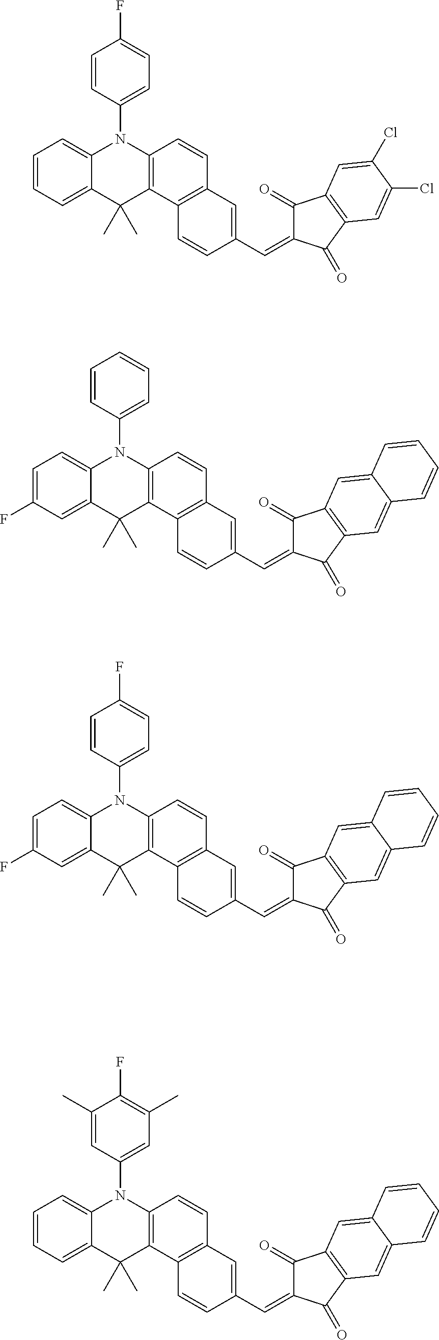 [SMILES: CC1(C)c2ccccc2N(c2ccc(F)cc2)c2ccc3cc(C=C4C(=O)c5cc(Cl)c(Cl)cc5C4=O)ccc3c21 ; CC1(C)c2cc(F)ccc2N(c2ccccc2)c2ccc3cc(C=C4C(=O)c5cc6ccccc6cc5C4=O)ccc3c21 ; CC1(C)c2cc(F)ccc2N(c2ccc(F)cc2)c2ccc3cc(C=C4C(=O)c5cc6ccccc6cc5C4=O)ccc3c21 ; Cc1cc(N2c3ccccc3C(C)(C)c3c2ccc2cc(C=C4C(=O)c5cc6ccccc6cc5C4=O)ccc32)cc(C)c1F]

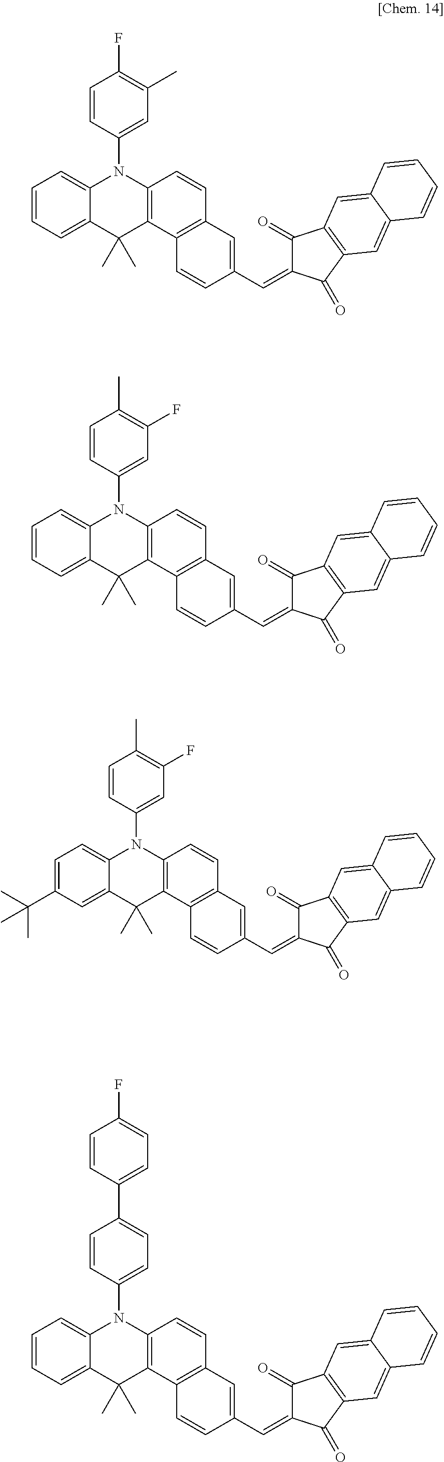 [SMILES: Cc1cc(N2c3ccccc3C(C)(C)c3c2ccc2cc(C=C4C(=O)c5cc6ccccc6cc5C4=O)ccc32)ccc1F ; Cc1ccc(N2c3ccccc3C(C)(C)c3c2ccc2cc(C=C4C(=O)c5cc6ccccc6cc5C4=O)ccc32)cc1F ; Cc1ccc(N2c3ccc(C(C)(C)C)cc3C(C)(C)c3c2ccc2cc(C=C4C(=O)c5cc6ccccc6cc5C4=O)ccc32)cc1F ; CC1(C)c2ccccc2N(c2ccc(-c3ccc(F)cc3)cc2)c2ccc3cc(C=C4C(=O)c5cc6ccccc6cc5C4=O)ccc3c21]

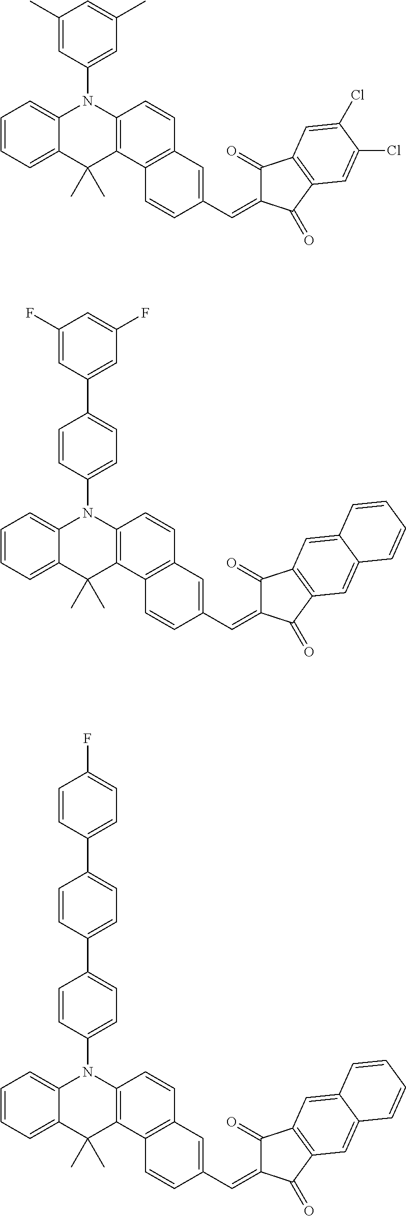 [SMILES: Cc1cc(C)cc(N2c3ccccc3C(C)(C)c3c2ccc2cc(C=C4C(=O)c5cc(Cl)c(Cl)cc5C4=O)ccc32)c1 ; CC1(C)c2ccccc2N(c2ccc(-c3cc(F)cc(F)c3)cc2)c2ccc3cc(C=C4C(=O)c5cc6ccccc6cc5C4=O)ccc3c21 ; CC1(C)c2ccccc2N(c2ccc(-c3ccc(-c4ccc(F)cc4)cc3)cc2)c2ccc3cc(C=C4C(=O)c5cc6ccccc6cc5C4=O)ccc3c21]

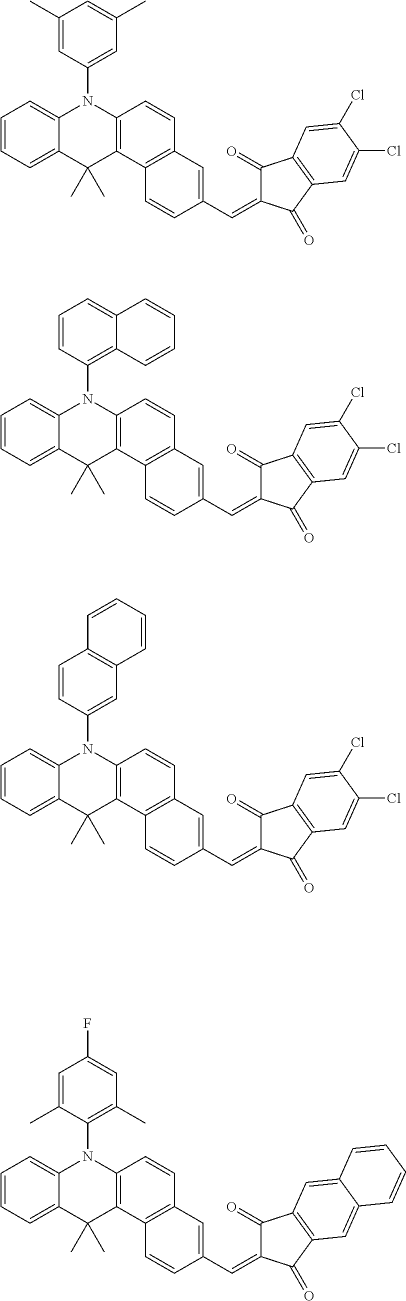 [SMILES: Cc1cc(C)cc(N2c3ccccc3C(C)(C)c3c2ccc2cc(C=C4C(=O)c5cc(Cl)c(Cl)cc5C4=O)ccc32)c1 ; CC1(C)c2ccccc2N(c2cccc3ccccc23)c2ccc3cc(C=C4C(=O)c5cc(Cl)c(Cl)cc5C4=O)ccc3c21 ; CC1(C)c2ccccc2N(c2ccc3ccccc3c2)c2ccc3cc(C=C4C(=O)c5cc(Cl)c(Cl)cc5C4=O)ccc3c21 ; Cc1cc(F)cc(C)c1N1c2ccccc2C(C)(C)c2c1ccc1cc(C=C3C(=O)c4cc5ccccc5cc4C3=O)ccc21]

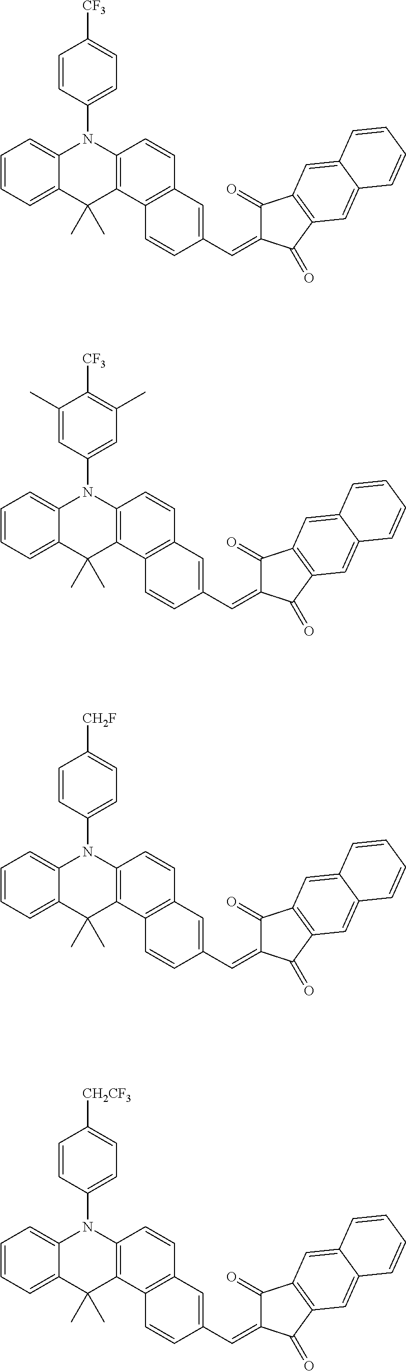

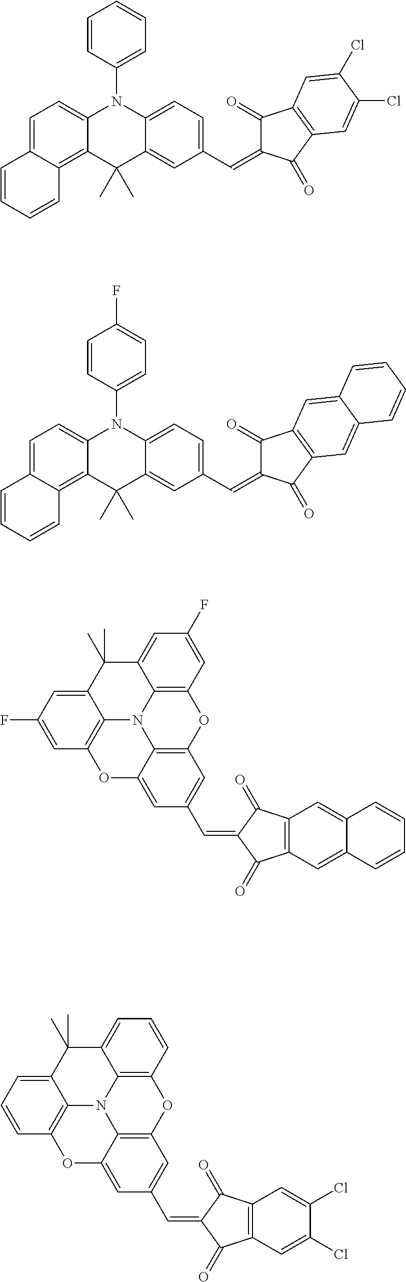 [SMILES: CC1(C)c2cc(C=C3C(=O)c4cc(Cl)c(Cl)cc4C3=O)ccc2N(c2ccccc2)c2ccc3ccccc3c21 ; CC1(C)c2cc(C=C3C(=O)c4cc5ccccc5cc4C3=O)ccc2N(c2ccc(F)cc2)c2ccc3ccccc3c21 ; CC1(C)c2cc(F)cc3c2N2c4c(cc(C=C5C(=O)c6cc7ccccc7cc6C5=O)cc4Oc4cc(F)cc1c42)O3 ; CC1(C)c2cccc3c2N2c4c(cc(C=C5C(=O)c6cc(Cl)c(Cl)cc6C5=O)cc4Oc4cccc1c42)O3]

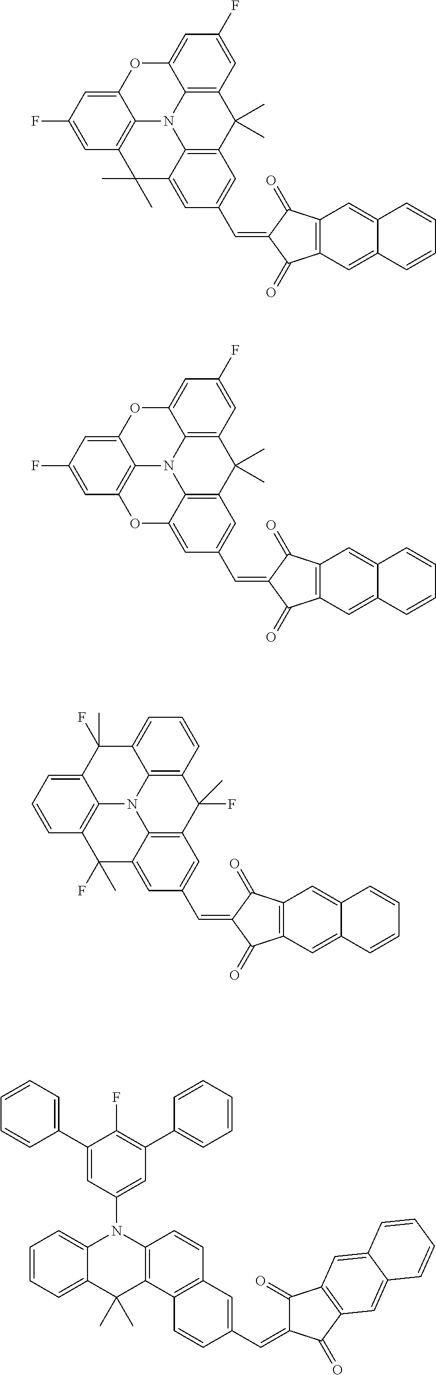 [SMILES: CC1(C)c2cc(F)cc3c2N2c4c(cc(F)cc4C(C)(C)c4cc(C=C5C(=O)c6cc7ccccc7cc6C5=O)cc1c42)O3 ; CC1(C)c2cc(F)cc3c2N2c4c(cc(F)cc4Oc4cc(C=C5C(=O)c6cc7ccccc7cc6C5=O)cc1c42)O3 ; CC1(F)c2cccc3c2N2c4c1cccc4C(C)(F)c1cc(C=C4C(=O)c5cc6ccccc6cc5C4=O)cc(c12)C3(C)F ; CC1(C)c2ccccc2N(c2cc(-c3ccccc3)c(F)c(-c3ccccc3)c2)c2ccc3cc(C=C4C(=O)c5cc6ccccc6cc5C4=O)ccc3c21]

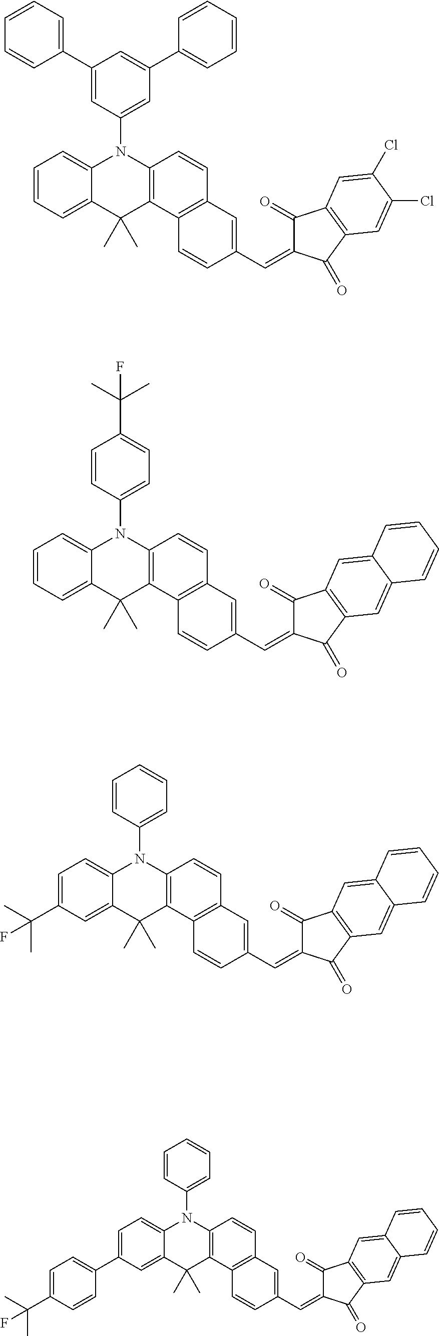 [SMILES: CC1(C)c2ccccc2N(c2cc(-c3ccccc3)cc(-c3ccccc3)c2)c2ccc3cc(C=C4C(=O)c5cc(Cl)c(Cl)cc5C4=O)ccc3c21 ; CC(C)(F)c1ccc(N2c3ccccc3C(C)(C)c3c2ccc2cc(C=C4C(=O)c5cc6ccccc6cc5C4=O)ccc32)cc1 ; CC(C)(F)c1ccc2c(c1)C(C)(C)c1c(ccc3cc(C=C4C(=O)c5cc6ccccc6cc5C4=O)ccc13)N2c1ccccc1 ; CC(C)(F)c1ccc(-c2ccc3c(c2)C(C)(C)c2c(ccc4cc(C=C5C(=O)c6cc7ccccc7cc6C5=O)ccc24)N3c2ccccc2)cc1]

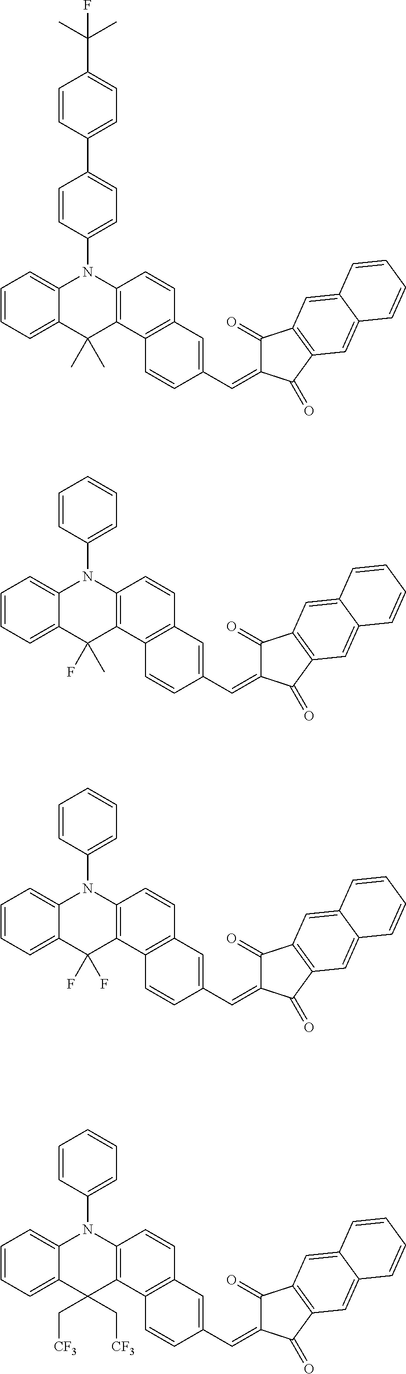 [SMILES: CC(C)(F)c1ccc(-c2ccc(N3c4ccccc4C(C)(C)c4c3ccc3cc(C=C5C(=O)c6cc7ccccc7cc6C5=O)ccc43)cc2)cc1 ; CC1(F)c2ccccc2N(c2ccccc2)c2ccc3cc(C=C4C(=O)c5cc6ccccc6cc5C4=O)ccc3c21 ; O=C1C(=Cc2ccc3c4c(ccc3c2)N(c2ccccc2)c2ccccc2C4(F)F)C(=O)c2cc3ccccc3cc21 ; O=C1C(=Cc2ccc3c4c(ccc3c2)N(c2ccccc2)c2ccccc2C4(CC(F)(F)F)CC(F)(F)F)C(=O)c2cc3ccccc3cc21]

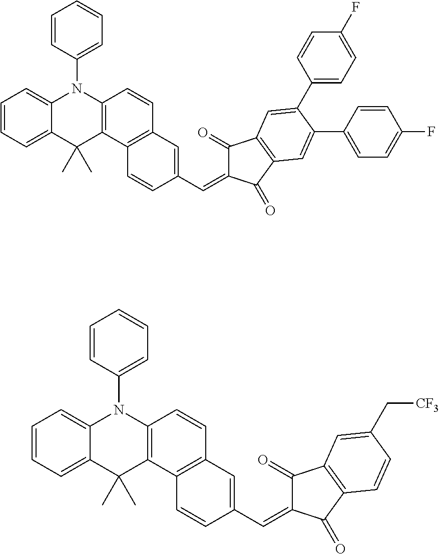 [SMILES: CC1(C)c2ccccc2N(c2ccccc2)c2ccc3cc(C=C4C(=O)c5cc(-c6ccc(F)cc6)c(-c6ccc(F)cc6)cc5C4=O)ccc3c21 ; CC1(C)c2ccccc2N(c2ccccc2)c2ccc3cc(/C=C4/C(=O)c5ccc(CC(F)(F)F)cc5C4=O)ccc3c21]

(Other Materials)

The photoelectric conversion layer may further contain a p-type organic compound or an n-type organic compound as a photoelectric conversion material.

The p-type organic semiconductor (compound) is a donor-type organic semiconductor (compound). This material is mainly represented by a hole-transporting organic compound and refers to an organic compound that easily donates electrons. More specifically, when two organic materials are used by being brought into contact to each other, an organic compound having a smaller ionization potential is called the p-type organic semiconductor. Accordingly, as the donor-type organic compound, any organic compounds can be used as long as they have electron-donating properties. For example, as the donor-type organic compound, it is possible to use a triarylamine compound, a benzidine compound, a pyrazoline compound, a styrylamine compound, a hydrazone compound, a triphenylmethane compound, a carbazole compound, and the like.

The n-type organic semiconductor (compound) is an acceptor-type organic semiconductor. This material is mainly represented by an electron-transporting organic compound and refers to an organic compound that easily accepts electrons. Since the n-type organic semiconductor is a semiconductor, conductors such as nanotubes, graphite and conductive polymers are not included therein. More specifically, when two organic compounds are used by being brought into contact to each other, an organic compound showing a higher degree of electron affinity is called the n-type organic semiconductor. Accordingly, as the acceptor-type organic semiconductor, any organic compounds can be used as long as they have electron-accepting properties. Preferable examples of the acceptor-type organic semiconductor include fullerene or fullerene derivatives, condensed aromatic carbon ring compounds (naphthalene derivatives, anthracene derivatives, phenanthrene derivatives, tetracene derivatives, pyrene derivatives, perylene derivatives, and fluoranthene derivatives), 5 to 7-membered heterocyclic compounds containing nitrogen atoms, oxygen atoms, or sulphur atoms (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, oxazole, indazole, benzimidazole, benzotriazole, benzoxazole, benzothiazole, carbazole, purine, triazolopyridazine, triazolopyrimidine, tetrazaindene, oxadiazole, imidazopyridine, pyrrolidine, pyrrolopyridine, thiadiazolopyridine, dibenzazepine, and tribenzazepine), a polyarylene compound, a fluorene compound, a cyclopentadiene compound, a silyl compound, metal complexes having nitrogen-containing heterocyclic compounds as ligands, and the like.

As the aforementioned n-type organic compound, fullerene or fullerene derivatives are preferable. Fullerene refers to fullerene C₆₀, fullerene C₇₀, fullerene C₇₆, fullerene C₇₈, fullerene C₈₀, fullerene C₈₂, fullerene C₈₄, fullerene C₉₀, fullerene C₉₆, fullerene C₂₄₀, fullerene C₅₄₀, or mixed fullerene, and fullerene derivatives refer to compounds obtained when a substituent is added to the fullerene. As the substituent of the fullerene derivatives, alkyl groups, aryl groups, or heterocyclic groups are preferable. As fullerene derivatives, the compounds disclosed in JP 2007-123707 A are preferable.

It is preferable for the photoelectric conversion layer to have a bulk-heterostructure which is formed in a state where the compound represented by Formula (1) is mixed with fullerene or fullerene derivatives. The bulk-heterostructure is the photoelectric conversion layer in which a p-type organic semiconductor (the compound represented by Formula (1)) and an n-type organic semiconductor are mixed together and dispersed. The structure can be formed by either a wet method or a dry method, but it is preferable to form the structure by a co-vapor deposition method. If a heterojunction structure is formed in the photoelectric conversion layer, it is possible to make up for a defect of a short carrier diffusion length of the photoelectric conversion layer and to improve the photoelectric conversion efficiency of the photoelectric conversion layer. The bulk-heterojunction structure is described in detail in paragraphs [0013] and [0014] of JP 2005-303266 A and the like.

In the photoelectric conversion layer, in view of a response speed, a ratio of a content of fullerene or derivatives thereof to a total content of fullerene or derivatives thereof and the compound represented by Formula (1) (a film thickness expressed in terms of a single layer of fullerene or derivatives thereof/(a film thickness expressed in terms of a single layer of the compound represented by Formula (1)+a film thickness expressed in terms of a single layer of fullerene or derivatives thereof)) is preferably equal to or higher than 50% by volume, and more preferably equal to or higher than 65% by volume.

The photoelectric conversion film containing the compound represented by Formula (1) of the present invention (an n-type organic compound may also be mixed into the film) is a non-light-emitting film, and has characteristics different from that of an organic electroluminescence device (OLED). The non-light-emitting film refers to a film having luminous quantum efficiency equal to or lower than 1%. The luminous quantum efficiency is preferably equal to or lower than 0.5%, and more preferably equal to or lower than 0.1%.

(Film Forming Method)

A photoelectric conversion layer 12 can be formed by a dry film formation method or a wet film formation method. Specific examples of the dry film formation method include physical vapor deposition methods such as a vacuum vapor deposition method, a sputtering method, an ion plating method, and an MBE method and CVD methods such as plasma polymerization. As the wet film formation method, a casting method, a spin coating method, a dipping method, an LB method, and the like are used. Among these, a dry film formation method is preferable, and a vacuum vapor deposition method is more preferable. When the vacuum vapor deposition method is used for forming a film, production conditions including a degree of vacuum and vapor deposition temperature can be set according to common methods.

The thickness of the photoelectric conversion layer 12 is preferably from 10 nm to 1,000 nm, more preferably from 50 nm to 800 nm, and particularly preferably from 100 nm to 500 nm. If the thickness of the photoelectric conversion layer 12 is 10 nm or more, a preferable effect of suppressing dark currents is obtained, and if the thickness of the photoelectric conversion layer 12 is 1,000 nm or less, preferable photoelectric conversion efficiency is obtained.

[Electrode]

The electrodes (the upper electrode (transparent conductive film) 15 and the lower electrode (conductive film) 11) are configured with a conductive material. As the conductive material, a metal, an alloy, a metal oxide, an electroconductive compound, a mixture of these, and the like can be used.

Since light enters the photoelectric conversion element from the upper electrode 15, the upper electrode 15 needs to be transparent enough to the light to be detected. Specific examples of the material of the upper electrode 15 include conductive metal oxides such as tin oxide doped with antimony, fluorine, or the like (ATO or FTC), tin oxide, zinc oxide, indium oxide, indium tin oxide (ITO), and indium zinc oxide (IZO), thin films of metal such as gold, silver, chromium, and nickel, mixtures or laminates composed of the metals and conductive metal oxides described above, inorganic conductive substances such as copper iodide and copper sulphide, organic conductive materials such as polyaniline, polythiophene, and polypyrrole, laminates composed of the organic conductive materials and ITO, and the like. Among these, in view of high conductivity, transparency, and the like, transparent conductive metal oxides are preferable.

When a transparent conductive film such as TCO (transparent conductive metal oxide) is used as the upper electrode 15, a DC short circuit or increase in a leakage current occurs in some cases. As one of the reasons, it is considered that fine cracks formed in the photoelectric conversion layer 12 may be covered with a dense film such as TCO, and accordingly, conduction may occur to a higher extent between the upper electrode 15 and the lower electrode 11 at the opposite side. Therefore, in the case of an electrode such as Al that is relatively poor in terms of film quality, increase in a leakage current does not easily occur. If a film thickness of the upper electrode 15 is controlled relative to a film thickness (that is, a depth of crack) of the photoelectric conversion layer 12, the increase in a leakage current can be suppressed to a large extent. A thickness of the upper electrode 15 is desirably controlled to be equal to or smaller than one fifth of a thickness of the photoelectric conversion layer 12, and more desirably controlled to be equal to or smaller than one tenth of a thickness of the photoelectric conversion layer 12.

Generally, when the thickness of the conductive film is decreased out of a certain range, a value of resistance rapidly increases. However, in a solid-state imaging device including the photoelectric conversion element according to the present embodiment, a sheet resistance may be preferably 100 Ω/square to 10,000 Ω/square, and the conductive film can be made into a thin film within a range of film thickness that can be set with a high degree of freedom. In addition, the thinner the upper electrode (transparent conductive film) 15 is, the smaller the amount of absorbed light becomes, and a light transmittance is increased in general. The increase in light transmittance is extremely preferable since amount of light absorbed into the photoelectric conversion layer 12 is increased, and thus a photoelectric conversion ability is enhanced. Considering the suppression of leakage current, increase in a value of resistance of the thin film, and increase in the transmittance that result from reduction of film thickness, the film thickness of the upper electrode 15 is preferably 5 nm to 100 nm, and more preferably 5 nm to 20 nm.

According to the use, sometimes transparency is provided to the lower electrode 11, or inversely, sometimes a light-reflecting material is used as the lower electrode 11 instead of providing transparency thereto. Specific examples of the material of the lower electrode 11 include conductive metal oxides such as tin oxide doped with antimony, fluorine, or the like (ATO or FTO), tin oxide, zinc oxide, indium oxide, indium tin oxide (ITO), and indium zinc oxide (IZO), metals such as gold, silver, chromium, nickel, titanium, tungsten, and aluminum, conductive compounds such as oxides or nitrides of the aforementioned metals (for example, titanium nitride (TiN)), mixtures or laminates composed of the aforementioned metals and conductive metal oxides, inorganic conductive substances such as copper iodide and copper sulfide, organic conductive materials such as polyaniline, polythiophene, and polypyrrole, laminates composed of the organic conductive materials and ITO or titanium nitride, and the like.

A method for forming the lower electrode 11 is not particularly limited, and can be appropriately selected according to the electrode material. Specifically, the lower electrode 11 can be formed by a wet method such as a printing method or a coating method, a physical method such as a vacuum vapor deposition method, a sputtering method, and an ion plating method, a chemical method such as CVD or a plasma CVD method, and the like.

When ITO is used as the electrode material, the lower electrode 11 can be formed by methods such as an electron beam method, a sputtering method, a resistance heating type vapor deposition method, a chemical reaction method (a sol-gel method or the like), and coating of a dispersion of indium tin oxide. Moreover, UV-ozone treatment, plasma treatment, or the like can be performed on the film prepared using ITO. When TiN is used as the electrode material, various methods including a reactive sputtering method are used, and UV-ozone treatment, plasma treatment, or the like can be further performed.

[Charge Blocking Layer: Electron Blocking Layer, Hole Blocking Layer]

The photoelectric conversion element of the present invention may have a charge blocking layer. If the photoelectric conversion element has such a layer, the characteristics (photoelectric conversion efficiency, response speed, and the like) of the obtained photoelectric conversion element are further improved. Examples of the charge blocking layer include an electron blocking layer and a hole blocking layer. Hereinafter, each of the layers will be described in detail.

(Electron Blocking Layer)

Electron-donating organic materials can be used for the electron blocking layer. Specifically, as low-molecular weight materials, it is possible to use aromatic diamine compounds such as N,N-bis(3-methylphenyl)-(1,1′-biphenyl)-4,4′-diamine (TPD) or 4,4′-bis[N-(naphthyl)-N-phenylamino]biphenyl (α-NPD), oxazole, oxadiazole, triazole, imidazole, imidazolone, stilbene derivatives, pyrazoline derivatives, tetrahydroimidazole, polyarylalkane, butadiene, 4,4′,4″-tris(N-(3-methylphenyl)N-phenylamino)triphenylamine (m-MTDATA), porphyrin compounds such as porphine, tetraphenylporphyrin copper, phthalocyanine, copper phthalocyanine, and titanium phthalocyanine oxide, triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, silazane derivatives, and the like. As high-molecular weight materials, it is possible to use polymers such as phenylene vinylene, fluorene, carbazole, indole, pyrene, pyrrole, picoline, thiophene, acetylene, and diacetylene and derivatives of these. The compounds that are not electron-donating compounds can also be used as long as they have sufficient hole transport properties.

Specifically, the compounds described in paragraphs [0083] to [0089] in JP 2008-72090 A are preferable.

It is preferable for the electron blocking layer to contain a compound represented by Formula (F-1). If such a compound is used, the response speed of the obtained photoelectric conversion film is further improved, and variance in the response speed between production rods can be further suppressed.

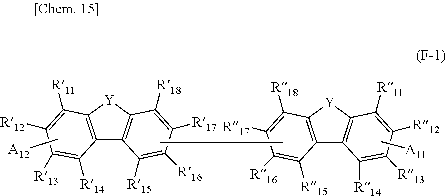

(In Formula (F-1), each of R″₁₁ to R″₁₈ and R′₁₁ to R′₁₈ independently represents a hydrogen atom, a halogen atom, an alkyl group, an aryl group, a heterocyclic group, a hydroxyl group, an amino group, or a mercapto group, and these may further have a substituent. One of R″₁₅ to R″₁₈ is linked to one of R′₁₅ to R′₁₈ and form a single bond. Each of A₁₁ and A₁₂ independently represents a group represented by the following Formula (A-1), and one of R″₁₁ to R″₁₄ and one of R′₁₁ to R′₁₄ are substituted with A₁₁ and A₁₂, respectively. Each Y independently represents a carbon atom, a nitrogen atom, an oxygen atom, a sulfur atom, or a silicon atom, and these may further have a substituent.)

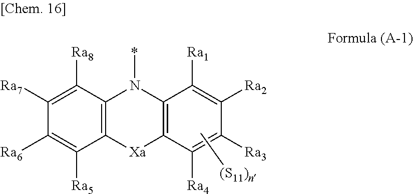

(In Formula (A-1), each of Ra₁ to Ra₈ independently represents a hydrogen atom, a halogen atom, an alkyl group, an aryl group, or a heterocyclic group, and these may further have a substituent. * represents a binding site. Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent. Each S₁₁ independently represents the following substituent (S₁₁), and one of Ra₁ to Ra₈ is substituted with the substituent (S₁₁). n′ represents an integer from 0 to 4.)

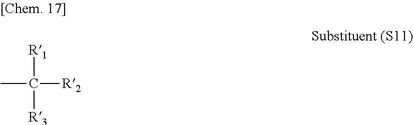

(Each of R′₁ to R′₃ independently represents a hydrogen atom or an alkyl group.)

In Formula (F-1), each of R″₁₁ to R″₁₈ and R′₁₁ to R′₁₈ independently represents a hydrogen atom, a halogen atom, an alkyl group, an aryl group, a heterocyclic group, a hydroxyl group, an amino group, or a mercapto group, and these may further have a substituent. Specific examples of the substituent include the substituent W described above. The substituent is preferably a halogen atom, an alkyl group, an aryl group, a heterocyclic group, a hydroxyl group, an amino group, or a mercapto group, more preferably a halogen atom, an alkyl group, an aryl group, or a heterocyclic group, even more preferably a fluorine atom, an alkyl group, or an aryl group, particularly preferably an alkyl group or an aryl group, and most preferably an alkyl group.

As R″₂₂ to R″₁₈ and R′₁₁ to R′₁₈, a hydrogen atom, and an alkyl group, an aryl group and a heterocyclic group that may have a substituent are preferable, and a hydrogen atom, and an alkyl group having 1 to 18 carbon atoms, an aryl group having 6 to 18 carbon atoms and a heterocyclic group having 4 to 16 carbon atoms that may have a substituent are more preferable. It is preferable that each of R″₁₂ and R′₁₂ be independently substituted with the substituent represented by Formula (A-1). It is more preferable that each of R″₁₂ and R′₁₂ be independently substituted with the substituent represented by Formula (A-1), and R″₁₁, R″₁₃ to R″₁₈, R′₁₁, and R′₁₃ to R′₁₈ be a hydrogen atom or an alkyl group having 1 to 18 carbon atoms that may have a substituent. It is particularly preferable that each of R″₁₂ and R′₁₂ be independently substituted with the substituent represented by Formula (A-1), and R″₁₁, R″₁₃ to R″₁₈, and R′₁₃ to R′₁₈ be a hydrogen atom.

Each Y independently represents a carbon atom, a nitrogen atom, an oxygen atom, a sulfur atom, or a silicon atom, and these may further have a substituent. That is, each Y represents a divalent linking group formed of a carbon atom, a nitrogen atom, an oxygen atom, a sulfur atom, or a silicon atom. As the divalent linking group, —C(R′₂₁)(R′₂₂)—, —Si(R′₂₃)(R′₂₄)—, and —N(R′₂₀)— are preferable, and —C(R′₂₁)(R′₂₂)— and —N(R′₂₀)— are more preferable, and —C(R′₂₁)(R′₂₂)— is particularly preferable.

Each of R′₂₀ to R′₂₄ independently represents a hydrogen atom, a halogen atom, an alkyl group, an aryl group, a heterocyclic group, a hydroxyl group, an amino group, or a mercapto group. The group constituting each of R′₂₀ to R′₂₄ may have a substituent, and specific examples of the substituent include the substituent W. As R′₂₀ to R′₂₄, a hydrogen atom, and an alkyl group, an aryl group and a heterocyclic group that may have a substituent are preferable; a hydrogen atom, and an alkyl group having 1 to 18 carbon atoms, an aryl group having 6 to 18 carbon atoms and a heterocyclic group having 4 to 16 carbon atoms that may have a substituent are more preferable; a hydrogen atom, and an alkyl group having 1 to 18 carbon atoms that may have a substituent are even more preferable; and an alkyl group having 1 to 18 carbon atoms is particularly preferable.

Each of Ra₁ to Ra₈ in Formula (A-1) independently represents a hydrogen atom, a halogen atom, an alkyl group, an aryl group, a heterocyclic group, a hydroxyl group, an amino group, or a mercapto group. The group constituting each of Ra₁ to Ra₈ may have a substituent, and specific examples of the substituent include the substituent W. Moreover, a plurality of substituents may form a ring by being bonded to each other.

As Ra₁ to Ra₈, a hydrogen atom, a halogen atom, an alkyl group having 1 to 18 carbon atoms, an aryl group having 6 to 18 carbon atoms, or a heterocyclic group having 4 to 16 carbon atoms is preferable; a hydrogen atom, an alkyl group having 1 to 12 carbon atoms, or an aryl group having 6 to 14 carbon atoms is more preferable; and a hydrogen atom, an alkyl group having 1 to 6 carbon atoms, or an aryl group having 6 to 10 carbon atoms is even more preferable. The alkyl group may be branched.

Specific examples preferable as Ra₁ to Ra₈ include a hydrogen atom, a methyl group, an ethyl group, a propyl group, a butyl group, a hexyl group, a cyclohexyl group, a phenyl group, and a naphthyl group.

Moreover, it is particularly preferable that Ra₃ and Ra₆ be a hydrogen atom or an alkyl group having 1 to 6 carbon atoms, and Ra₁, Ra₂, Ra₄, Ra₅, Ra₇, and Ra₈ be a hydrogen atom.

Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent.

Xa is preferably a single bond, an alkylene group having 1 to 12 carbon atoms, an alkenylene group having 2 to 12 carbon atoms, an arylene group having 6 to 14 carbon atoms, a heterocyclic group having 4 to 13 carbon atoms, an oxygen atom, a sulfur atom, an imino group (for example, a phenylimino group, a methylimino group, or a t-butylimino group) that has a hydrocarbon group (preferably an aryl group or an alkyl group) having 1 to 12 carbon atoms, or a silylene group, more preferably a single bond, an oxygen atom, an alkylene group (for example, a methylene group, an 1,2-ethylene group, or a 1,1-dimethylmethylene group) having 1 to 6 carbon atoms, an alkenylene group (for example, —CH₂═CH₂—) having 2 carbon atoms, an arylene group (for example, a 1,2-phenylene group or a 2,3-naphthylene group) having 6 to 10 carbon atoms, or a silylene group, and even more preferably a single bond, an oxygen atom, an alkylene group (for example, a methylene group, a 1,2-ethylene group, or a 1,1-dimethylmethylene group) having 1 to 6 carbon atoms. These may further have the substituent W as a substituent.

Specific examples of the group represented by Formula (A-1) include the groups exemplified in the following N1 to N11, but the present invention is not limited thereto. As the group represented by Formula (A-1), N-1 to N-7 are preferable, N-1 to N-6 are more preferable, N-1 to N-3 are even more preferable, N-1 and N-2 are particularly preferable, and N-1 is most preferable.

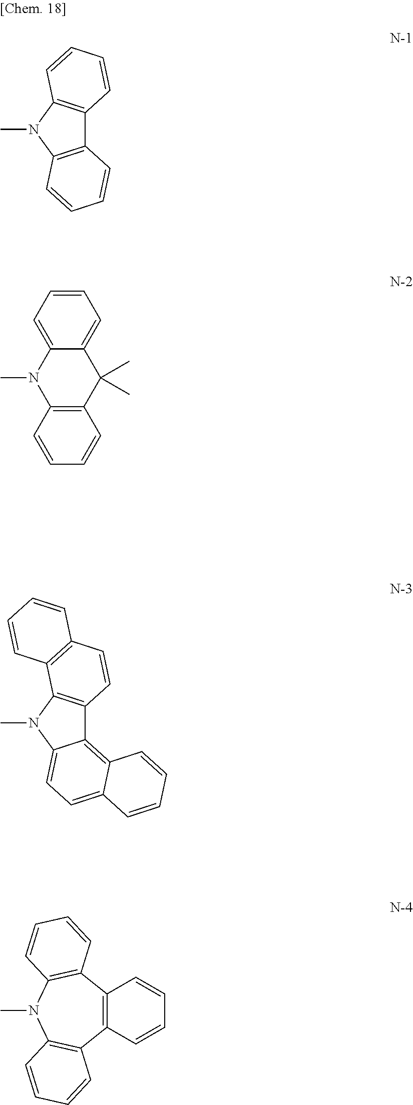

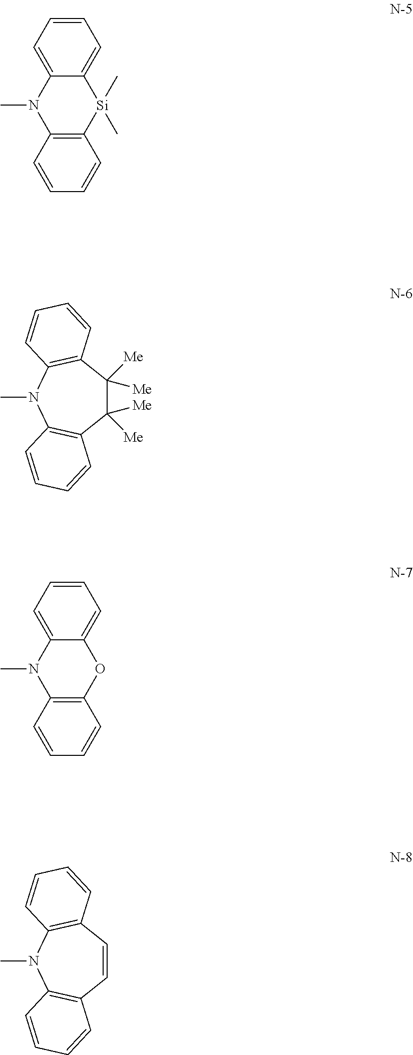

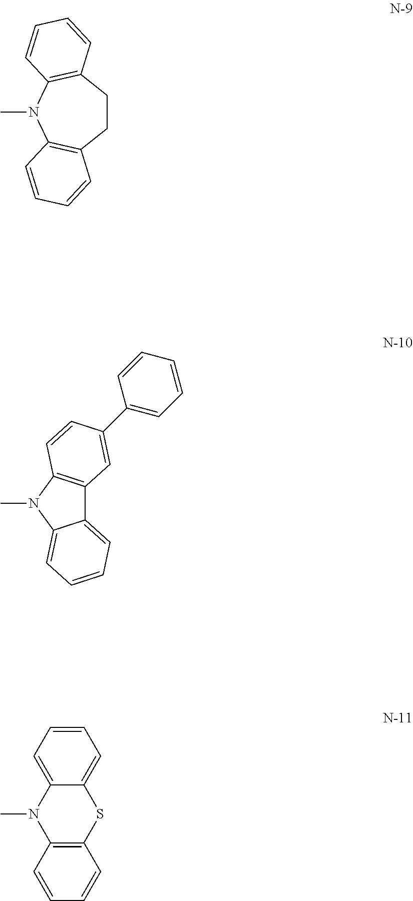 [SMILES: CN1c2ccccc2CCc2ccccc21 ; Cn1c2ccccc2c2cc(-c3ccccc3)ccc21 ; CN1c2ccccc2Sc2ccccc21]

In the substituent (S₁₁), R′₁ represents a hydrogen atom or an alkyl group. As R′₁, a methyl group, an ethyl group, a propyl group, an iso-propyl group, a butyl group, or a tert-butyl group is preferable; a methyl group, an ethyl group, a propyl group, an iso-propyl group, or a tert-butyl group is more preferable; a methyl group, an ethyl group, an iso-propyl group, or a tert-butyl group is even more preferable; and a methyl group, an ethyl group, or a tert-butyl group is particularly preferable.

R′₂ represents a hydrogen atom or an alkyl group. As R′₂, a hydrogen atom, a methyl group, an ethyl group, a propyl group, an iso-propyl group, a butyl group, or a tert-butyl group is preferable; a hydrogen atom, a methyl group, an ethyl group, or a propyl group is more preferable; a hydrogen atom or a methyl group is more preferable; and a methyl group is particularly preferable.

R′₃ represents a hydrogen atom or an alkyl group. As R′₃, a hydrogen atom or a methyl group is preferable, and a methyl group is more preferable.

R′₁ to R′₃ may form a ring by being bonded to each other respectively. When a ring is formed, the number of ring member is not particularly limited. However, the ring is preferably a 5- or 6-membered ring, and more preferably a 6-membered ring.

S₁₁ represents the substituent (S₁₁) described above, and one of Ra₁ to Ra₈ is substituted with the substituent (S₁₁). It is preferable for one of Ra₃ and Ra₆ in Formula (A-1) to represent the substituent (S₁₁).

Preferable examples of the substituent (S₁₁) include the following (a) to (x). Among these, (a) to (j) are preferable, (a) to (h) are more preferable, (a) to (f) are even more preferable, (a) to (c) are particularly preferable, and (a) is most preferable.

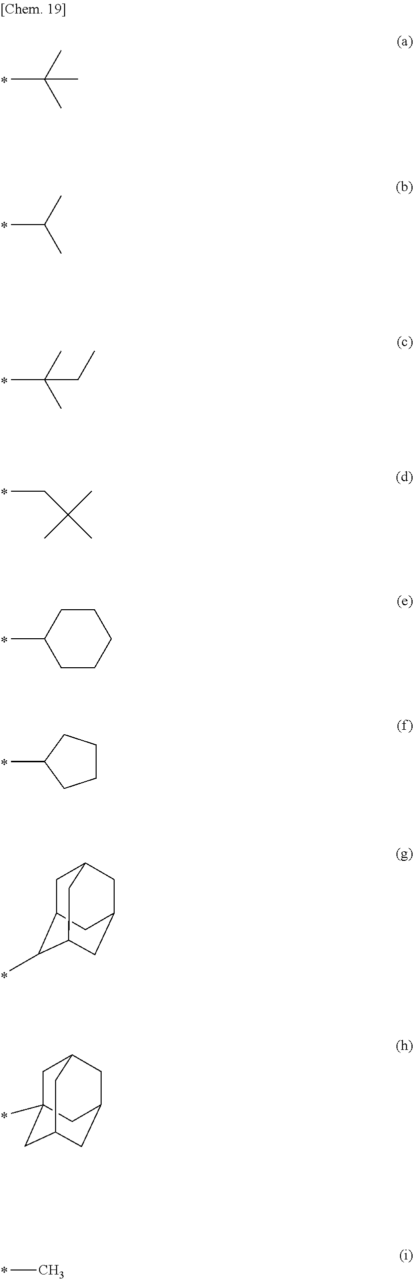

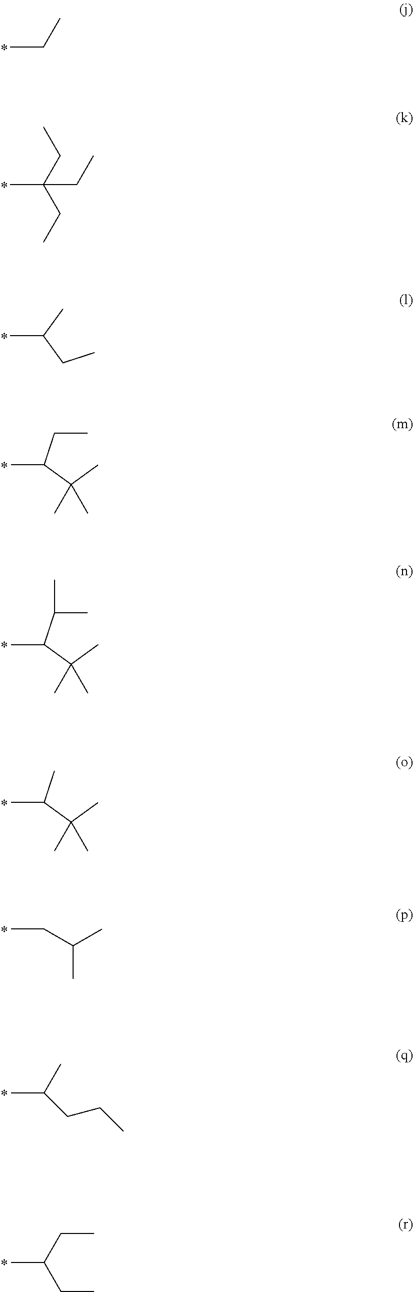

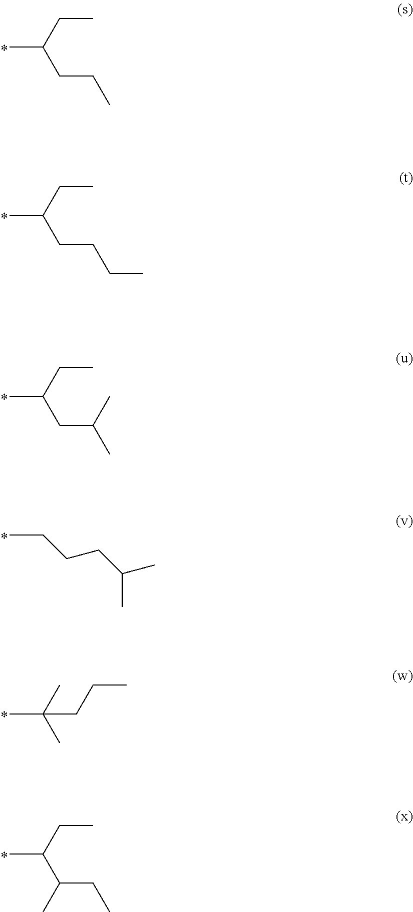

Each n′ independently represents an integer from 0 to 4. n′ is preferably 0 to 3, more preferably 0 to 2, even more preferably 1 to 2, and particularly preferably 2.

The Formula (A-1) may be a group represented by the following Formula (A-3), a group represented by the following Formula (A-4), or a group represented by the following Formula (A-5).

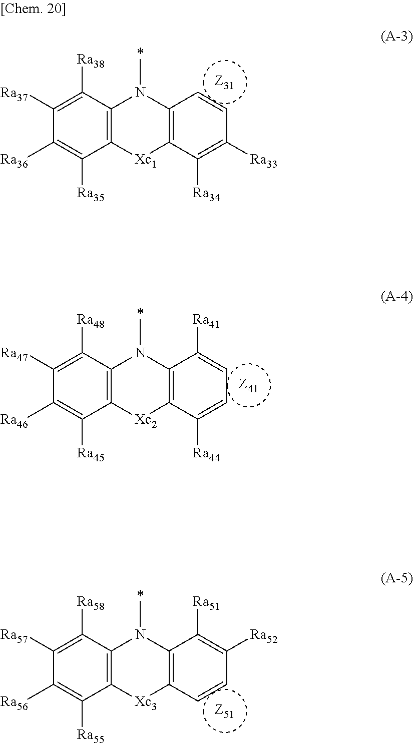

(In Formulae (A-3) to (A-5), each of Ra₃₃ to Ra₃₈, Ra₄₁, Ra₄₄ to Ra₄₈, Ra₅₁, Ra₅₂, and Ra₅₅ to Ra₅₈ independently represents a hydrogen atom, a halogen atom, or an alkyl group, and these may further have a substituent. * represents a binding site. Each of Xc₁, Xc₂, and Xc₃ independently represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent. Each of Z₃₁, Z₄₁, and Z₅₁ independently represents a cycloalkyl ring, an aromatic hydrocarbon ring, or an aromatic heterocycle, and these may further have a substituent.)

In Formulae (A-3) to (A-5), each of Ra₃₃ to Ra₃₈, Ra₄₁, Ra₄₄ to Ra₄₈, Ra₅₁, Ra₅₂, and Ra₅₅ to Ra₅₈ independently represents a hydrogen atom, a halogen atom (preferably a fluorine atom, a chlorine atom, a bromine atom, or an iodine atom), or an alkyl group. Among these, a hydrogen atom or an alkyl group is preferable, and a hydrogen atom is more preferable, since a substituent with low polarity is advantageous for transporting holes.

When Ra₃₃ to Ra₃₈, Ra₄₁, Ra₄₄ to Ra₄₈, Ra₅₁, Ra₅₂, and Ra₅₅ to Rasa represent an alkyl group, the alkyl group is preferably an alkyl group having 1 to 18 carbon atoms, more preferably an alkyl group having 1 to 12 carbon atoms, and even more preferably an alkyl group having 1 to 6 carbon atoms. Specifically, a methyl group, an ethyl group, a propyl group, a butyl group, a hexyl group, and a cyclohexyl group are preferable.

In Formulae (A-3) to (A-5), among Ra₃₃ to Ra₃₈, Ra₃₈, Ra₄₄ to Ra₄₈, Ra₅₁, Ra₅₂, and Ra₅₅ to Ra₅₈, the groups adjacent to each other may form a ring by being bonded to each other, and examples of the ring include the ring R described above. As the ring, a benzene ring, a naphthalene ring, an anthracene ring, a pyridine ring, a pyrimidine ring, and the like are preferable.

Each of Xc₁, Xc₂, and Xc₃ independently represents a single bond, an oxygen atom, a sulfur atom, an alkylene group, a silylene group, an alkenylene group, a cycloalkylene group, a cycloalkenylene group, an arylene group, a divalent heterocyclic group, or an imino group. When Xc₁, Xc₂, and Xc₃ represent an alkylene group, a silylene group, an alkenylene group, a cycloalkylene group, a cycloalkenylene group, an arylene group, a divalent heterocyclic group, or an imino group, these may further have a substituent. Examples of the substituent include the substituent W.

Xc₁, Xc₂, and Xc₃ are preferably a single bond, an alkylene group having 1 to 12 carbon atoms, an alkenylene group having 2 to 12 carbon atoms, an arylene group having 6 to 14 carbon atoms, a heterocyclic group having 4 to 13 carbon atoms, an oxygen atom, a sulfur atom, or an imino group (for example, a phenylimino group, a methylimino group, or a t-butylimino group) having a hydrocarbon group (preferably an aryl group or an alkyl group) having 1 to 12 carbon atoms, and more preferably a single bond, an alkylene group (for example, a methylene group, a 1,2-ethylene group, or a 1,1-dimethylmethylene group) having 1 to 6 carbon atoms, an alkenylene group (for example, —CH₂═CH₂—) having 2 carbon atoms, or an arylene group (for example, a 1,2-phenylene group or a 2,3-naphthylene group) having 6 to 10 carbon atoms.

Each of Z₃₁, Z₄₁, and Z₅₁ independently represents a cycloalkyl ring, an aromatic hydrocarbon ring, or an aromatic heterocycle. In Formulae (A-3) to (A-5), Z₃₁, Z₄₁, and Z₅₁ are condensed with a benzene ring. Z₃₁, Z₄₁, and Z₅₁ are preferably an aromatic hydrocarbon ring, since the photoelectric conversion element is expected to exhibit high heat resistance and excellent hole transporting ability.

The electron blocking layer may configured with plural layers.

As the electron blocking layer, inorganic materials can also be used. Generally, inorganic materials have a higher dielectric constant compared to organic materials. Accordingly, when inorganic materials are used for the electron blocking layer, higher voltage is applied to the photoelectric conversion layer, hence the photoelectric conversion efficient can be improved. Examples of materials that can form the electron blocking layer include calcium oxide, chromium oxide, copper-chromium oxide, manganese oxide, cobalt oxide, nickel oxide, copper oxide, copper-gallium oxide, copper-strontium oxide, niobium oxide, molybdenum oxide, copper-indium oxide, silver-indium oxide, iridium oxide, and the like.

(Hole Blocking Layer)

For the hole blocking layer, an electron-accepting organic material can be used.

As the electron-accepting material, it is possible to use oxadiazole derivatives such as 1,3-bis(4-tert-butylphenyl-1,3,4-oxadiazolyl)phenylene (OXD-7), anthraquinodimethane derivatives, diphenylquinone derivatives, bathocuproin, bathophenanthroline, derivatives of these, a triazole compound, a tris(8-hydroxyquinolinato)aluminum complex, a bis(4-methyl-8-quinolinato)aluminum complex, distyrylarylene derivatives, a silole compound, and the like. Moreover, materials other than the electron-accepting organic material can also be used as long as the materials exhibit sufficient electron transporting ability. For example, a porphyrin-based compound, a styryl-based compound such as DCM (4-dicyanomethylene-2-methyl-6-(4-(dimethylaminostyryl))-4H pyran), a 4H pyran-based compound can be used. Specifically, the compounds described in paragraphs [0073] to [0078] of JP 2008-72090 A are preferable.

A method for producing the charge blocking layer is not particularly limited, and the charge blocking layer can be formed by a dry film formation method or a wet film formation method. As the dry film formation method, it is possible to use a vapor deposition method, a sputtering method, and the like. The vapor deposition may be physical vapor deposition (PVD) or chemical vapor deposition (CVD), but among these, physical vapor deposition such as vacuum vapor deposition is preferable. As the wet film formation method, it is possible to use an inkjet method, a spraying method, a nozzle printing method, a spin coating method, a dip coating method, a casting method, a die coating method, a roll coating method, a bar coating method, a gravuer coating method, and the like. Among these, from the viewpoint of high-accuracy patterning, an inkjet method is preferable.

A thickness of each of the charge blocking layers (the electron blocking layer and the hole blocking layer) is preferably 10 nm to 200 nm, more preferably 30 nm to 150 nm, and particularly preferably 50 nm to 100 nm. This is because if the thickness is too small, the dark current suppressing effect is diminished, and if it is too great, the photoelectric conversion efficiency decreases.

[Substrate]

The photoelectric conversion element may further include substrate. The type of substrate to be used is not particularly limited, and it is possible to use a semiconductor substrate, a glass substrate, or a plastic substrate.

The position of the substrate is not particularly limited. Generally, on the substrate, a conductive film, a photoelectric conversion film, and a transparent conductive film are laminated on one another in this order.

[Sealing Layer]

The photoelectric conversion element may further include a sealing layer. In the presence of factors such as water molecules that deteriorate the photoelectric conversion material, performance of the material markedly deteriorates in some cases. If the entire photoelectric conversion film is covered and sealed with a dense sealing layer that does not allow water molecules to permeate the film, such as ceramics like metal oxide, metal nitride, and metal nitride oxide or diamond-like carbon (DLC), the deterioration described above can be prevented.

The selection of material of the sealing layer and production of the sealing layer may be performed according to the description in paragraphs [0210] to [0215] of JP 2011-082508 A.

[Photosensor]

The photoelectric conversion element of the present invention can be used as, for example, a solar cell or a photosensor. It is preferable for the photoelectric conversion element of the present invention to be used as a photosensor. The photosensor may use only a single photoelectric conversion element. Alternatively, the photosensor may be preferably in the form of a line sensor in which the Photoelectric conversion elements are arranged in a straight line, or in the form of a two-dimensional sensor in which the photoelectric conversion elements are arranged on a plane. In a line sensor, the photoelectric conversion element of the present invention functions as an imaging device by converting optical image information into electric signals by using an optical system and a driving portion just like a scanner. In a two-dimensional sensor, the photoelectric conversion element of the present invention functions as an imaging device by converting optical image information into electric signal by forming an image on the sensor by using an optical system just like an imaging module.

A photoelectric cell is a power-generating apparatus. Accordingly, efficiency in converting light energy into electric energy is regarded as an important performance, but dark currents which are electric currents generated in a dark place do not cause a problem for the performance thereof. Moreover, the photoelectric cell does not require a heating process at the late stage such as installation of a color filter. For the photosensor, conversion of luminance signals into electric signals with a high accuracy is regarded as an important performance, and consequentially, efficiency in converting amount of light into electric current is also an important performance. However, when being output in a dark place, the signals become noise, and accordingly, a low level of dark currents is required. Furthermore, the resistance to the process of a late stage is also important.

[Imaging Device]

Next, an example of a configuration of an imaging device having the photoelectric conversion element 10a will be described.

In the example of a configuration described below, members and the like having the same configuration and function as the members and the like which have already been described are marked with the same signs or corresponding signs in the drawing, and description thereof is simplified or skipped.

An imaging device is a device that converts optical information of an image into electric signals. In this device, plural photoelectric conversion elements are arranged on a matrix in the same plane. An optical signal is converted into an electric signal in each of the photoelectric conversion elements (pixels), and the electric signal can be sequentially output outside the imaging device for each image. Accordingly, each pixel is configured with one photoelectric conversion element and one or more transistors.

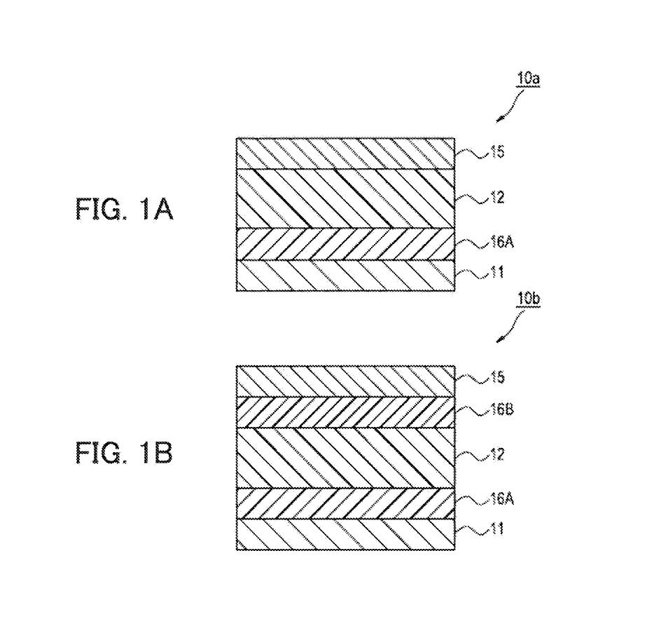

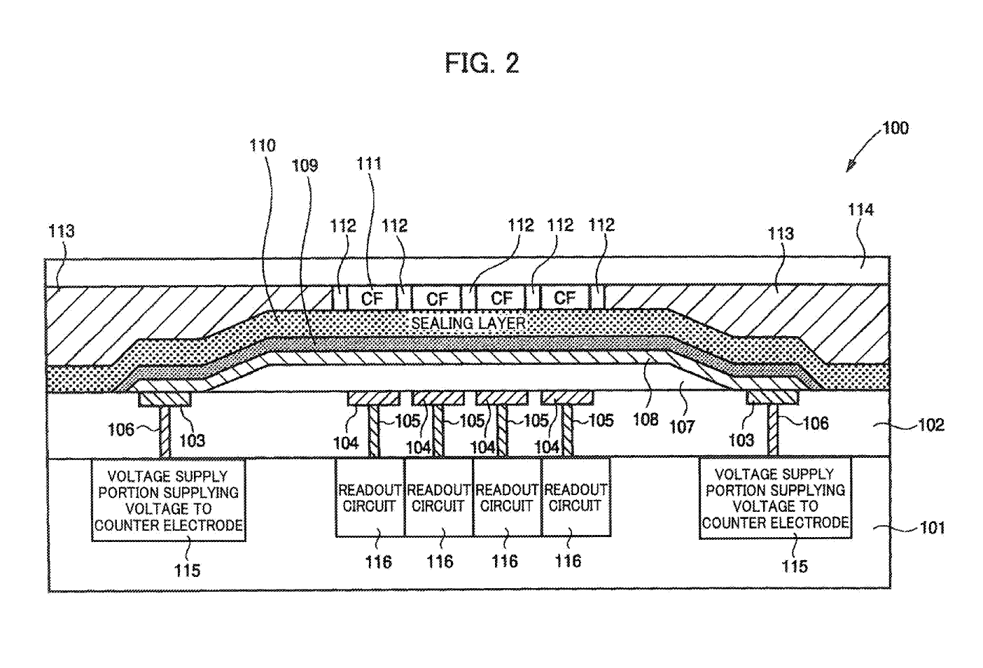

FIG. 2 is a cross-sectional view showing a schematic configuration of an imaging device for describing an embodiment of the present invention. The imaging device is used by being mounted on imaging apparatuses such as digital cameras or digital video cameras, electronic endoscopes, imaging modules of cellular phones, and the like.

The imaging device has plural photoelectric conversion elements shown in FIG. 1, and a circuit board on which readout circuits that read out signals corresponding to the charge generated by the photoelectric conversion film of each photoelectric conversion element are formed. The imaging device has a configuration in which plural photoelectric conversion elements are arranged one-dimensionally or two-dimensionally on the same plane positioned above the circuit board.

An imaging device 100 shown in FIG. 2 has a substrate 101, an insulating layer 102, a connection electrode 103, a pixel electrode (lower electrode) 104, a connection portion 105, a connection Portion 106, a photoelectric conversion film 107, a counter electrode (upper electrode) 108, a buffer layer 109, a sealing layer 110, a color filter (CF) 111, a partition 112, a light-shielding layer 113, a protective layer 114, a voltage supply portion 115 supplying voltage to the counter electrode, and a readout circuit 116.

The pixel electrode 104 has the same function as the electrode 11 of the photoelectric conversion element 10a shown in FIG. 1. The counter electrode 108 has the same function as the electrode 15 of the photoelectric conversion element 10a shown in FIG. 1. The photoelectric conversion film 107 has the same configuration as the layer disposed between the electrode 11 and the electrode 15 of the photoelectric conversion element 10a shown in FIG. 1.

The substrate 101 is a glass substrate or a semiconductor substrate such as Si. The insulating layer 102 is formed on the substrate 101. On the surface of the insulating layer 102, plural pixel electrodes 104 and plural connection electrodes 103 are formed.

The photoelectric conversion film 107 is a film that is disposed on plural pixel electrodes 104 to cover the electrodes and shared by all of the photoelectric conversion elements.

The counter electrode 108 is an electrode that is disposed on the photoelectric conversion film 107 and shared by all of the photoelectric conversion elements. The counter electrode 108 is formed such that it reaches the top of the connection electrode 103 disposed outside the photoelectric conversion film 107, and is electrically connected to the connection electrode 103.

The connection portion 106 is embedded in the insulating layer 102, and is plug or the like which is for electrically connecting the connection electrode 103 to the voltage supply portion 115 that supplies voltage to the counter electrode. The voltage supply portion 115 is formed in the substrate 101, and applies predetermined voltage to the counter electrode 108 through the connection portion 106 and the connection electrode 103. When the voltage that should be applied to the counter electrode 108 is higher than the power supply voltage of the imaging device, the voltage supply portion 115 supplies the predetermined voltage by increasing the power supply voltage by using a boosting circuit such as a charge pump.

The readout circuit 116 is disposed in the substrate 101 in an association with each of the plural pixel electrodes 104, and reads out the signal corresponding to the charge collected by the corresponding pixel electrode 104. The readout circuit 116 is configured with, for example, a CCD, CMOS, or TFT circuit, and is shielded from light by a light-shielding layer (not shown in the drawing) disposed inside the insulating layer 102. The readout circuit 116 is electrically connected to the pixel electrode 104 corresponding thereto through the connection portion 105.

The buffer layer 109 is formed on the counter electrode 108 while covering the counter electrode 108. The sealing layer 110 is formed on the buffer layer 109 while covering the buffer layer 109. The color filter 111 is formed on the sealing layer 110, in a position facing each pixel electrode 104. The partition 112 is disposed between color filters 111 and is for increasing light transmitting efficiency of the color filter 111.

The light-shielding layer 113 is formed on the sealing layer 110, in a position outside the region in which the color filter 111 and the partition 112 are disposed. The light-shielding layer 113 prevents light from entering the photoelectric conversion film 107 that is formed in a position outside an effective pixel region. The protective layer 114 is formed on the color filter 111, the partition 112, and the light-shielding layer 113 and protects the entire imaging device 100.

When light enters the imaging device 100 configured as above, the light enters the photoelectric conversion film 107, and charges are generated in the film. Among the generated charges, holes are collected by the pixel electrode 104, and voltage signals corresponding to the amount of holes are output outside the imaging device 100 by the readout circuit 116.

A method for producing the imaging device 100 is as follows.

On the circuit board in which the voltage supply portion 115 that supplies voltage to the counter electrode and the readout circuit 116 have been formed, the connection portions 105 and 106, plural connection electrodes 103, plural pixel electrodes 104, and the insulating layer 102 are formed. The plural pixel electrodes 104 are arranged on the surface of the insulating layer 102, in the form of, for example, a square lattice.

Thereafter, on the plural pixel electrodes 104, the photoelectric conversion film 107 is formed by, for example, a vacuum heating vapor deposition method. Then, on the photoelectric conversion film 107, the counter electrode 108 is formed by, for example, a sputtering method in a vacuum. Subsequently, on the counter electrode 108, the buffer layer 109 and the sealing layer 110 are formed in this order by, for example, a vacuum heating vapor deposition method. Thereafter, the color filter 111, the partition 112, and the light-shielding layer 113 are formed, and then the protective layer 114 is formed, and as a result, the imaging device 100 is completed.

In the method for producing the imaging device 100, if a process of placing the imaging device 100, which is being prepared, in a non-vacuum environment is added between the process of forming the photoelectric conversion film 107 and the process of forming the sealing layer 110, it is possible to prevent performance deterioration of the plural photoelectric conversion elements. If such a process is added, it is possible to reduce the production cost while preventing performance deterioration of the imaging device 100.

### Examples

Examples will be described below, but the present invention is not limited thereto.

(Synthesis of Compound D1)

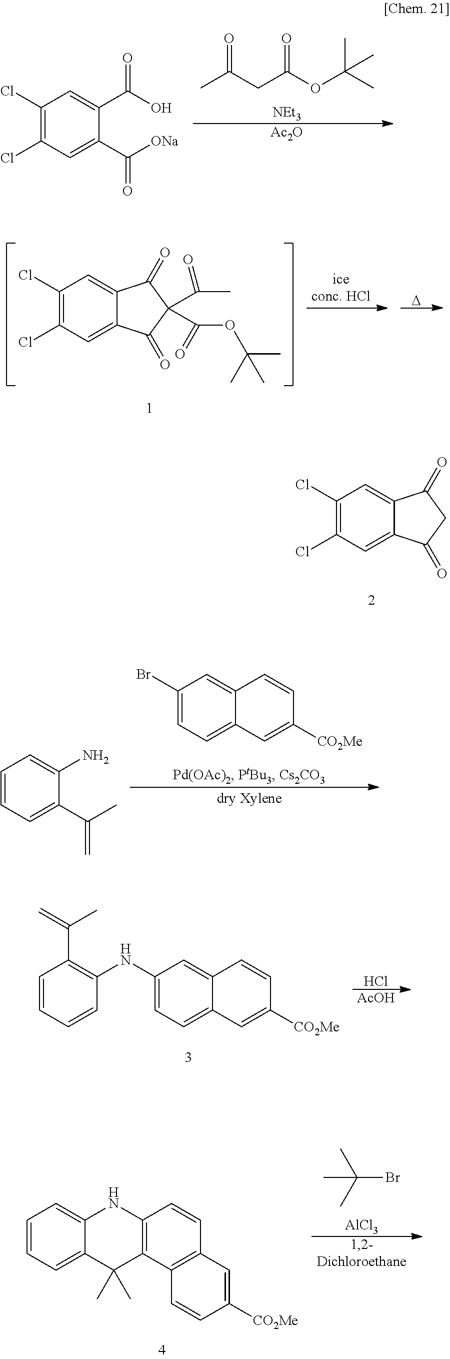

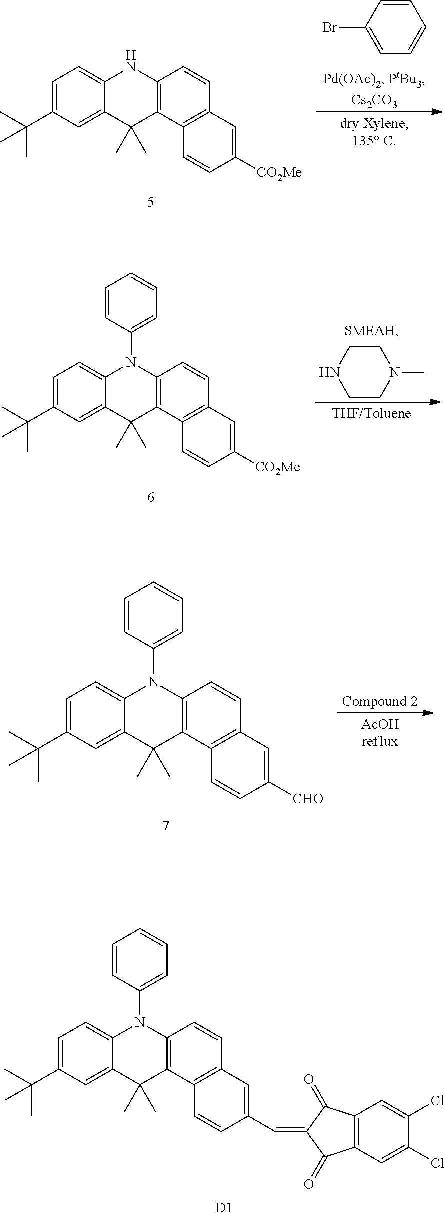

Sodium 4,5-dichlorophthalate (5.9 g, 23.0 mmol) and triethylamine (5.4 g, 53.0 mmol) were dissolved in 15 ml of acetic anhydride, and to the resultant solution, tert-butyl acetate (4.2 g, 26.5 mmol) was added dropwise. Thereafter, the solution was stirred for 2 hours at room temperature, thereby obtaining Compound 1. In a state where Compound 1 has not been isolated, 10 g of ice and 8 ml of 35% concentrated hydrochloric acid were directly added to the reaction mixture, and the resultant was heated and stirred for 30 minutes at 50° C., thereby obtaining Compound 2 at a yield of 83%.

2-iso-propenylaniline (4.20 g, 31.5 mmol), palladium acetate (210 mg, 0.95 mmol), tri(t-butyl)phosphine (570 mg, 2.80 mmol), cesium carbonate (20.5 g, 62.9 mmol), and methyl 6-bromo-2-naphthoate (8.35 g, 31.5 mmol) were dissolved in 50 ml of xylene and reacted under reflux at a boiling point for 5 hours in a nitrogen atmosphere, thereby obtaining Compound 3 at a yield of 83%. Compound 3 (7.80 g, 24.6 mmol) was added to a mixed solvent consisting of 40 ml of acetic acid and 8 ml of hydrochloric acid, and the resultant was stirred for 3 hours at 60° C., thereby obtaining Compound 4 at a yield of 82%. Compound 4 (5.0 g, 15.6 mol) and aluminum chloride (2.3 g, 17.4 mmol) were dissolved in 50 ml of 1,2-dichloroethane, and to the resultant, t-butylbromide (4.3 g, 31.5 mmol) was added dropwise at room temperature in a nitrogen atmosphere. The reaction mixture was stirred for 2 hours at 70° C., thereby obtaining Compound 5 at a yield of 98%. Compound 5 (1.00 g, 3.15 mmol), palladium acetate (70.7 mg, 0.315 mmol), tri(t-butyl)phosphine (191 mg, 0.945 mmol), cesium carbonate (2.05 g, 6.30 mmol), and bromobenzene (490 mg, 3.15 mmol) were dissolved in 15 ml of xylene and reacted under reflux at a boiling point for 7 hours in a nitrogen atmosphere, thereby obtaining Compound 6 at yield of 95%. In a nitrogen atmosphere, a 70% toluene solution of sodium bis(2-methoxyethoxy)aluminum dihydride (5.37 ml, 18.6 mmol) was added to 13 ml of THF, and the resultant was cooled to 0° C. N-methylpiperazine (2.01 g, 20.1 mmol) was added dropwise thereto and stirred for 30 minutes, thereby preparing a reductant solution. In a nitrogen atmosphere, the reductant solution was added dropwise to 20 ml of a THF solution of Compound 6 (1.35 g, 3.00 mmol) at −40° C. After the reaction solution was stirred for 4 hours at −20° C., the reaction was stopped using diluted hydrochloric acid, thereby obtaining Compound 7 at a yield of 81%. In a nitrogen atmosphere, Compound 7 (700 mg, 1.67 mmol) and Compound 2 (395 mg, 1.84 mmol) were added to 9 ml of an acetic acid solvent, and the resultant was subjected to reflux for 3 hours. After being left to cool, the resultant was subjected to suction filtration and recrystallized over N,N-dimethylacetamide. The resultant was subjected to suction filtration, thereby obtaining Compound D1 at a yield of 61%.

(Synthesis of Compound D2)

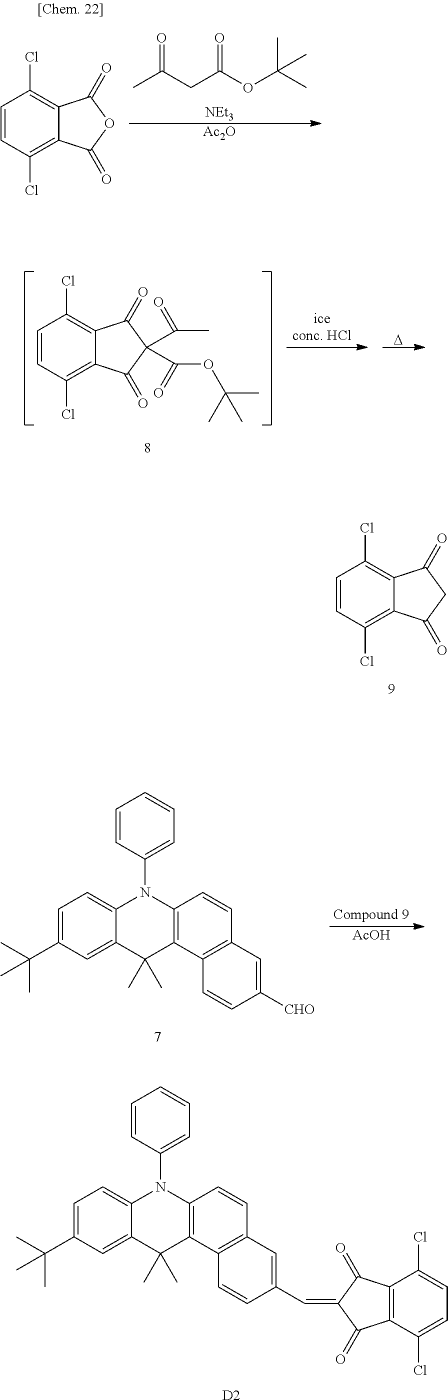

Compound 9 was obtained according to the same procedure as the synthesis method of Compound D1, except that 3,6-dichlorophthalic anhydride was used instead of sodium 4,5-dichlorophthalate. Moreover, Compound D2 was obtained at a yield of 59% according to the same procedure as the synthesis method of Compound D1, except that Compound 9 was used instead of Compound 2.

(Synthesis of Compound D3)

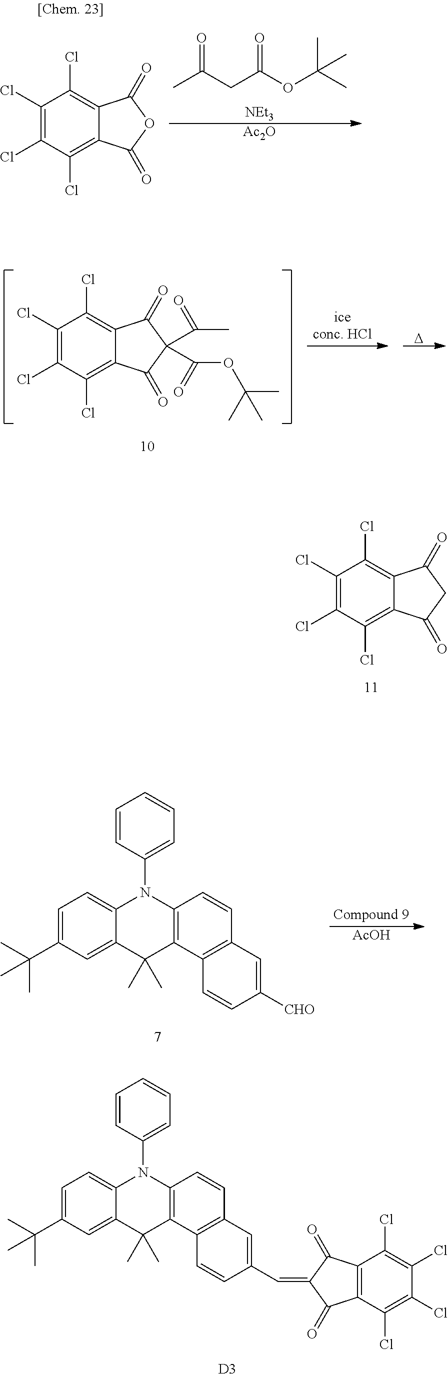

Compound 11 was obtained according to the same procedure as the synthesis method of Compound D1, except that 3,4,5,6-tetrachlorophthalic anhydride was used instead of sodium 4,5-dichlorophthalate. Moreover, Compound D3 was obtained at a yield of 68% according to the same procedure as the synthesis method of Compound D1, except that Compound 11 was used instead of Compound 2.

(Synthesis of Compound D4)

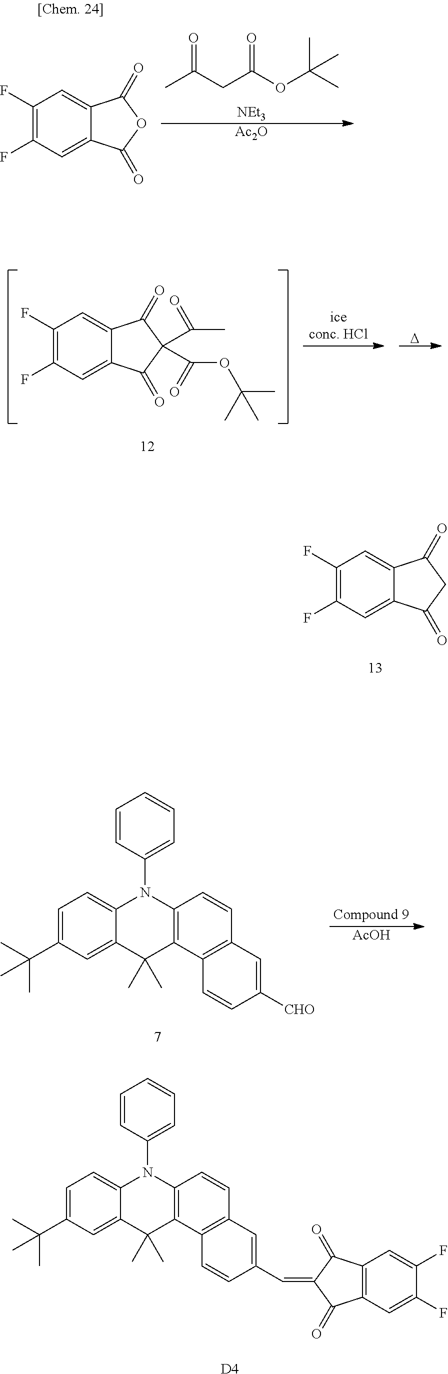

Compound 13 was obtained according to the same procedure as the synthesis method of Compound D1, except that 4,5-difluorophthalic anhydride was used instead of sodium 4,5-dichlorophthalate. Moreover, Compound D4 was obtained at a yield of 54% according to the same procedure as the synthesis method of Compound D1, except that Compound 13 was used instead of Compound 2.

(Synthesis of Compound D5)

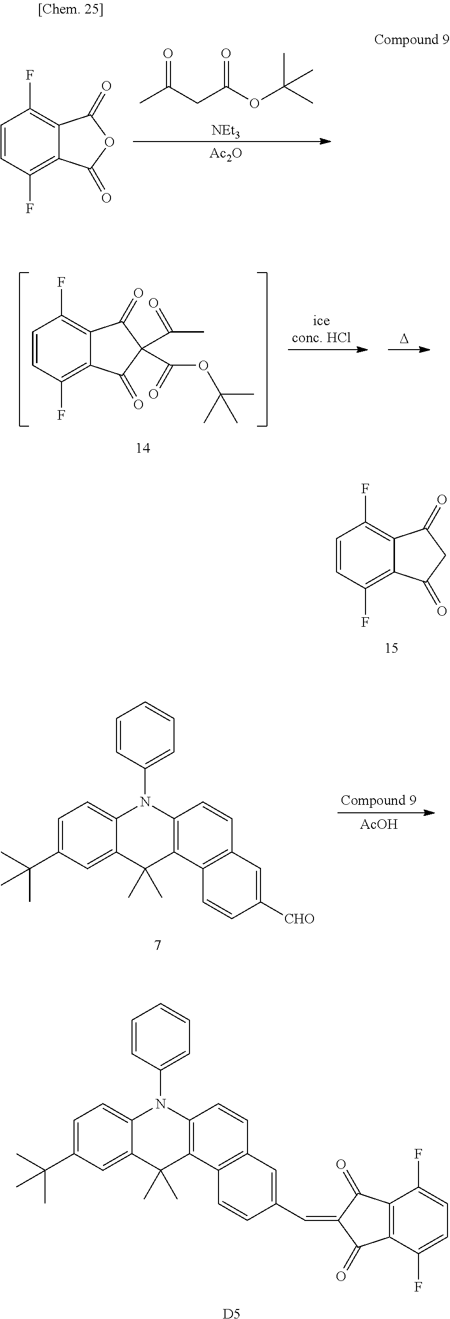

Compound 15 was obtained according to the same procedure as the synthesis method of Compound D1, except that 3,6-difluorophthalic anhydride was used instead of sodium 4,5-dichlorophthalate. Moreover, Compound D5 was obtained at a yield of 50% according to the same procedure as the synthesis method of Compound D1, except that Compound 15 was used instead of Compound 2.

(Synthesis of Compound D6)

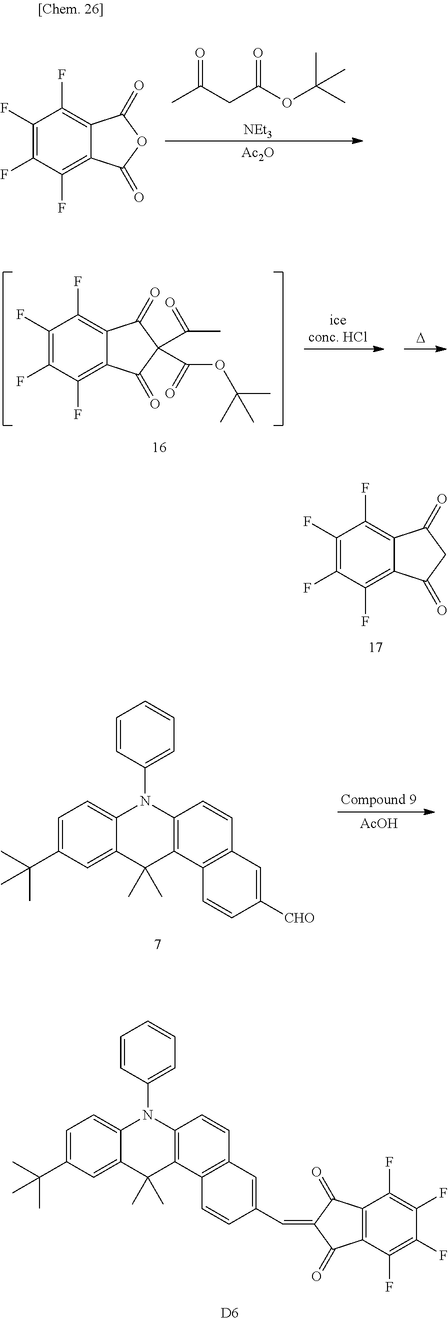

Compound 17 was obtained according to the same procedure as the synthesis method of Compound D1, except that 3,4,5,6-tetrafluorophthalic anhydride was used instead of sodium 4,5-dichlorophthalate. Moreover, Compound D6 was obtained at a yield of 63% according to the same procedure as the synthesis method of Compound D1, except that Compound 17 was used instead of Compound 2.

(Synthesis of Compound D7)

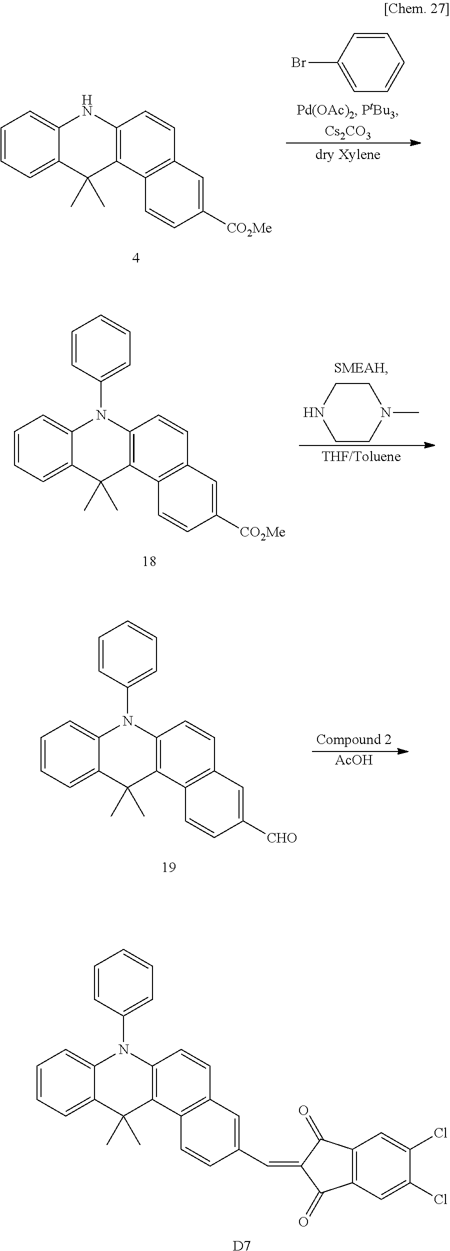

Compound 4 (1.00 g, 3.15 mmol), palladium acetate (70.7 mg, 0.315 mmol), tri(t-butyl)phosphine (191 mg, 0.945 mmol), cesium carbonate (2.05 g, 6.30 mmol), and bromobenzene (645 mg, 3.47 mmol) were dissolved in 15 ml of xylene and reacted under reflux at a boiling point for 7 hours in a nitrogen atmosphere, thereby obtaining Compound 18 at a yield of 89%. In a nitrogen atmosphere, a 70% toluene solution of sodium bis(2-methoxyethoxy)aluminum dihydride (SMEAH) (5.01 ml, 17.4 mmol) was added to 12 ml of THF, and the resultant was cooled to 0° C. N-methylpiperazine (1.88 g, 18.8 mmol) was added dropwise thereto, followed by stirring for 30 minutes, thereby preparing a reductant solution. In a nitrogen atmosphere, the reductant solution was added dropwise to 19 ml of a THF solution of Compound 18 (1.18 g, 2.80 mmol) at −40° C. After the reaction solution was stirred for 4 hours at −20° C., the reaction was stopped using diluted hydrochloric acid, thereby obtaining Compound 19 at a yield of 70%. In a nitrogen atmosphere, Compound 19 (712 mg, 1.96 mmol) and Compound 2 (464 mg, 2.16 mmol) were added to 15 ml of an acetic acid solvent, and the resultant was subjected to reflux for 3 hours. After being left to cool, the resultant was subjected to suction filtration and recrystallized over N,N-dimethylacetamide. By performing suction filtration, Compound D7 was obtained at a yield of 61%.

(Synthesis of Compound D8)

Compound D8 was obtained at a yield of 57% according to the same procedure as the synthesis method of Compound D7, except that Compound 9 was used instead of Compound 2.

(Synthesis of Compound D9)

Compound D9 was obtained at a yield of 67% according to the same procedure as the synthesis method of Compound D7, except that Compound 11 was used instead of Compound 2.

(Synthesis of Compound D10)

Compound D10 was obtained at a yield of 55% according to the same procedure as the synthesis method of Compound D7, except that Compound 13 was used instead of Compound 2.

(Synthesis of Compound D11)

Compound D11 was obtained at a yield of 52% according to the same procedure as the synthesis method of Compound D7, except that Compound 15 was used instead of Compound 2.

(Synthesis of Compound D12)

Compound D12 was obtained at a yield of 61% according to the same procedure as the synthesis method of Compound D7, except that Compound 17 was used instead of Compound 2.

(Synthesis of Compound D13)

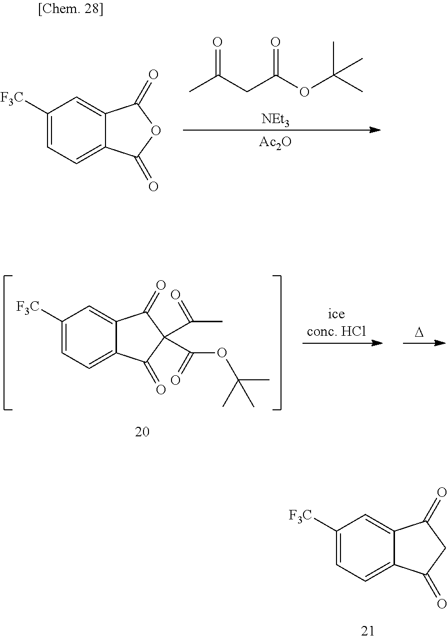

Compound 21 was obtained according to the same procedure as the synthesis method of Compound D1, except that 5-trifluoromethylphthalic anhydride was used instead of sodium 4,5-dichlorophthalate. Moreover, Compound D13 was obtained at a yield of 46% according to the same procedure as the synthesis method of Compound D7, except that Compound 21 was used instead of Compound 2.

(Synthesis of Compound D14)

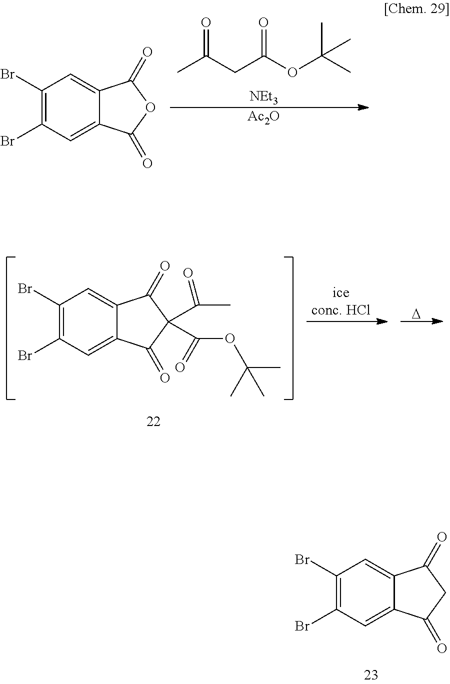

Compound 23 was obtained according to the same procedure as the synthesis method of Compound D1, except that 4,5-dibromophthalic anhydride was used instead of sodium 4,5-dichlorophthalate. Moreover, Compound D14 was obtained at a yield of 70% according to the same procedure as the synthesis method of Compound D7, except that Compound 23 was used instead of Compound 2.

(Synthesis of Compound D15)

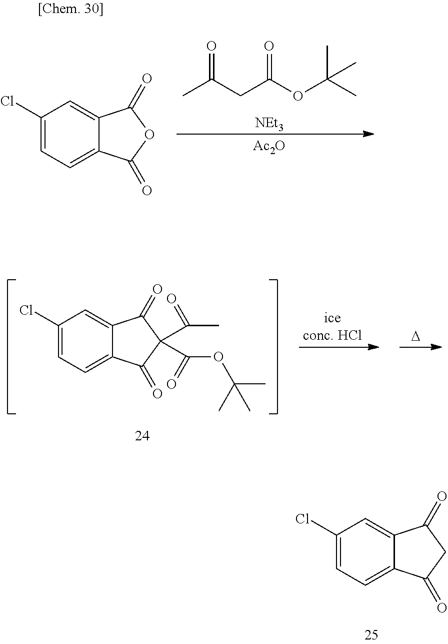

Compound 25 was obtained according to the same procedure as the synthesis method of Compound D1, except that 5-chlorophthalic anhydride was used instead of sodium 4,5-dichlorophthalate. Moreover, Compound D15 was obtained at a yield of 43% according to the same procedure as the synthesis method of Compound D7, except that Compound 25 was used instead of Compound 2.

(Synthesis of Compound D16)

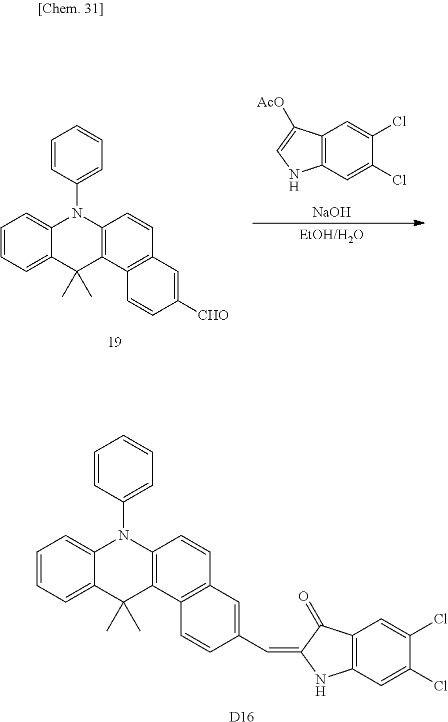

In a nitrogen atmosphere, acetic acid-4,5-dichloroindoxyl (0.90 g, 3.7 mmol) and sodium hydroxide (1.5 g, 37.5 mmol) were added to 20 ml of water as a solvent, and the resultant was heated under reflux for 1 hour. To the resultant, 20 ml of an ethanol solution of Compound 19 (1.34 g, 3.7 mmol) was added dropwise over 10 minutes, and the resultant was heated under reflux for 2 hours. After being left to cool, the resultant was subjected to suction filtration and recrystallized over N,N-dimethylacetamide. By performing suction filtration, Compound D16 was obtained at a yield of 12%.

(Synthesis of Compound D17)

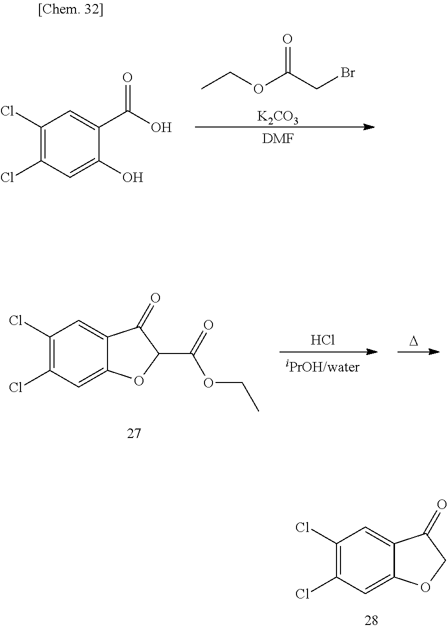

In a nitrogen atmosphere, 3-hydroxy-4-naphthoate (10.0 g, 53.1 mmol), ethyl bromoacetate (22.2 g, 132.9 mmol), and potassium carbonate (36.4 g, 265.5 mmol) were added to 100 ml of DMF, and the resultant was heated and stirred for 5 hours at 80° C., thereby obtaining Compound 27 at a yield of 56%. Compound 27 (5.0 g, 19.5 mmol) and 10 ml of 35% concentrated hydrochloric acid were added to a mixed solvent consisting of 30 ml of isopropanol and 20 ml of water, and the resultant was heated and stirred for 3 hours at 80° C. The resultant was left to cool, and then the precipitate was filtered and dried, thereby obtaining Compound 28 at a yield of 75%. Moreover, Compound 17 was obtained at a yield of 67% according to the same procedure as the synthesis method of Compound D7, except that Compound 28 was used instead of Compound 2.

(Synthesis of Compound D18)

Compound D18 was obtained at a yield of 54% according to the same procedure as the synthesis method of Compound D7, except that 6,7-dichlorobenzoindanedione was used instead of Compound 2.

(Synthesis of Compound D19)

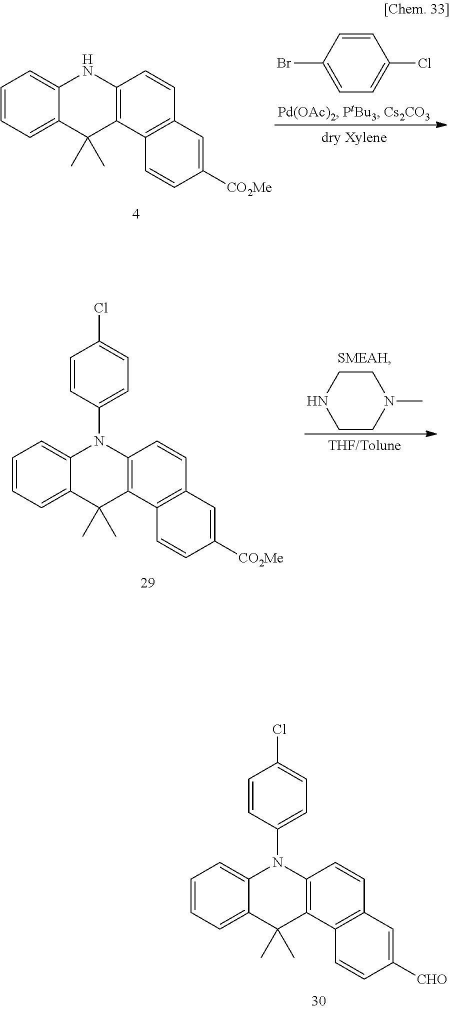

Compound 30 was synthesized according to the same procedure as the synthesis method of Compound D7, except that 4-bromochlorobenzene was used instead of bromobenzene. Moreover, Compound D19 was obtained at a yield of 56% according to the same procedure as the synthesis method of Compound D7, except that Compound 30 was used instead of Compound 19, and benzoindanedione was used instead of Compound 2.

(Synthesis of Compound D20)

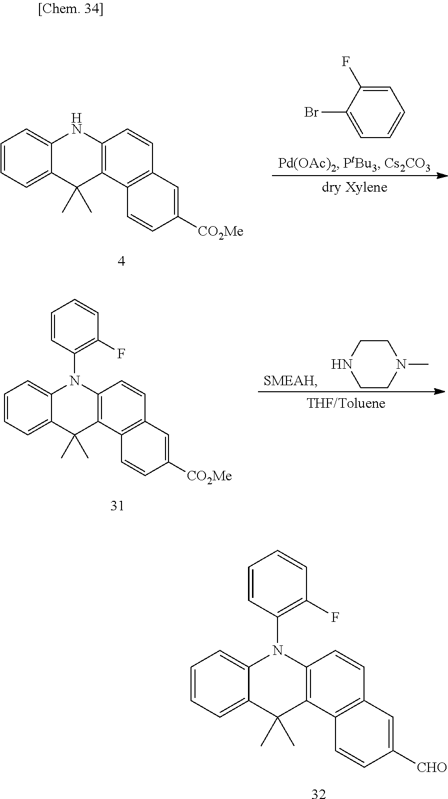

Compound 32 was synthesized according to the same procedure as the synthesis method of Compound D7, except that 2-bromofluorobenzene was used instead of bromobenzene. Moreover, Compound D20 was obtained at a yield of 50% according to the same procedure as the synthesis method of Compound D7, except that Compound 32 was used instead of Compound 19, and benzoindanedione was used instead of Compound 2.

(Synthesis of Compound D21)

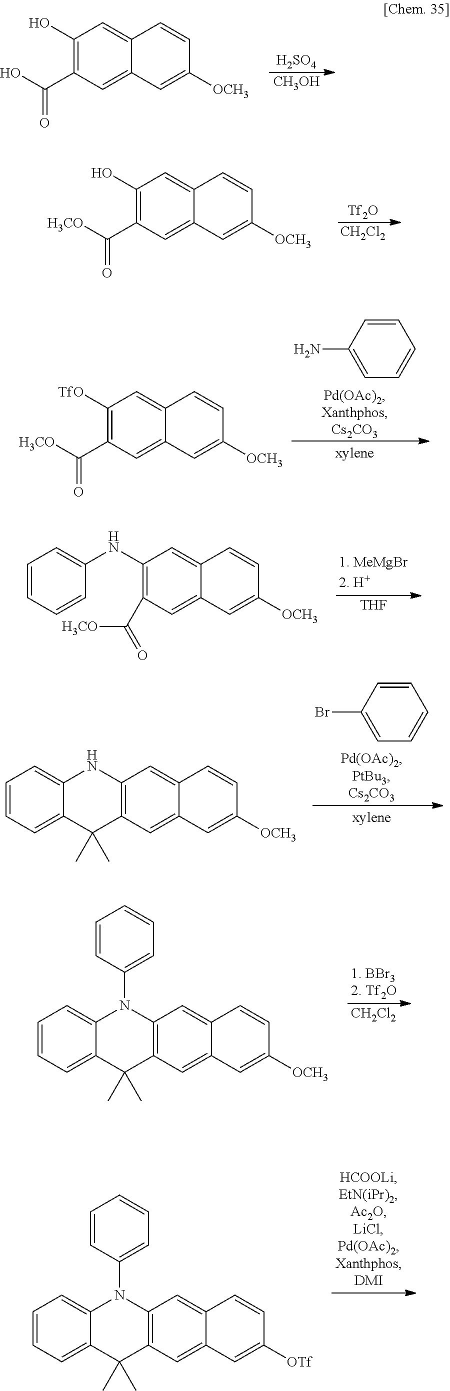

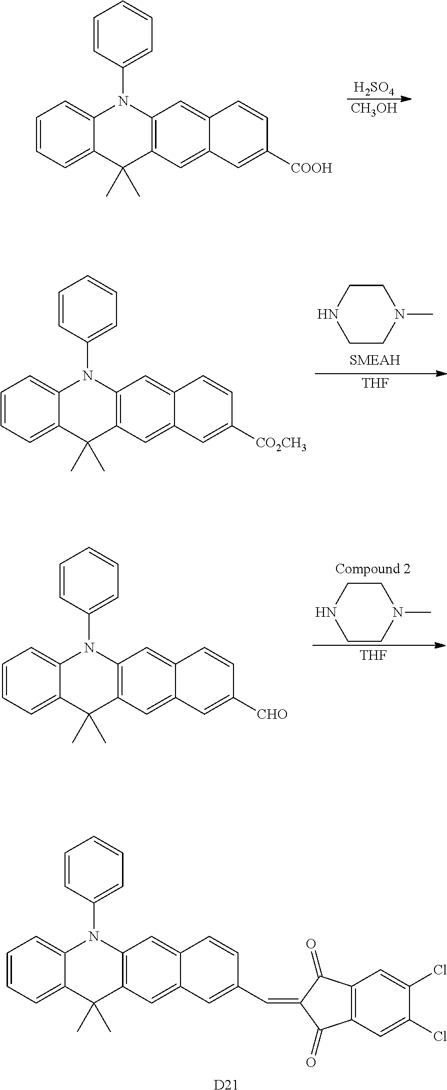

Compound D21 was synthesized according to the above scheme.

(Synthesis of Compound D22)

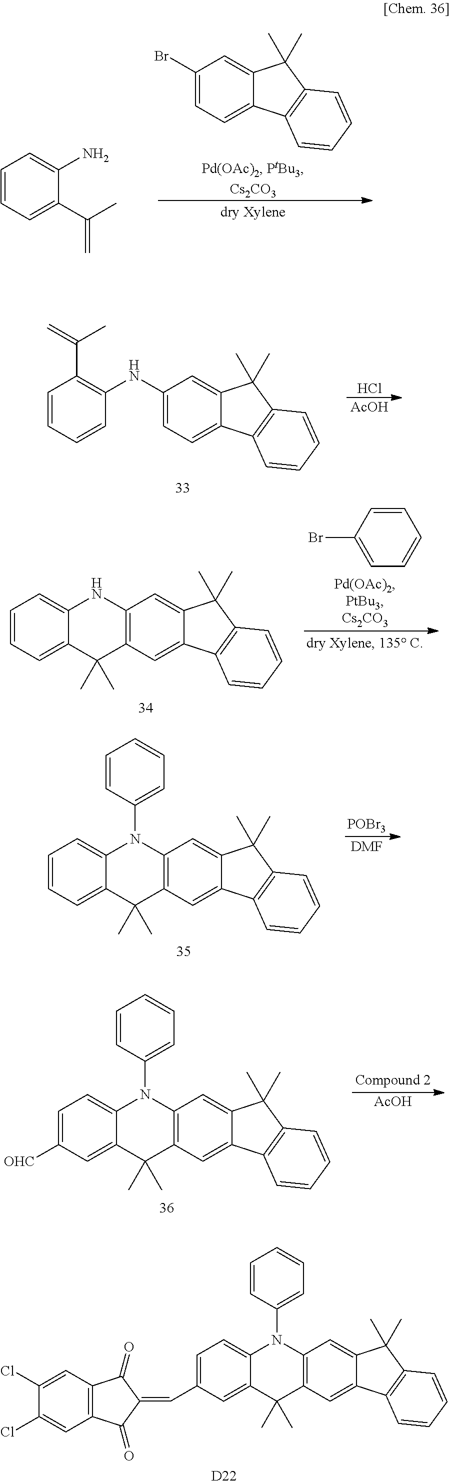

2-iso-propenylaniline (4.20 g, 31.5 mmol), palladium acetate (210 mg, 0.95 mmol), tri(t-butyl)phosphine (570 mg, 2.80 mmol), cesium carbonate (20.5 g, 62.9 mmol), and 2-bromo-9,9-dimethylfluorene (11.1 g, 31.5 mmol) were dissolved in 50 ml of xylene and reacted under reflux at a boiling point for 5 hours in a nitrogen atmosphere, thereby obtaining Compound 33 at a yield of 73%. Compound 33 (7.48 g, 23.0 mmol) was added to a mixed solvent consisting of 35 ml of acetic acid and 7 ml of hydrochloric acid, and the resultant was stirred for 30 minutes at 60° C., thereby obtaining Compound 34 at a yield of 81%. Compound 34 (3.25 g, 10.0 mmol), palladium acetate (112 mg, 0.500 mmol), tri(t-butyl)phosphine (303 mg, 1.50 mmol), cesium carbonate (6.52 g, 20.0 mmol), and bromobenzene (1.73 g, 11.0 mmol) were dissolved in 40 ml of xylene and reacted under reflux at a boiling point for 7 hours in a nitrogen atmosphere, thereby obtaining Compound 35 at a yield of 75%. In a nitrogen atmosphere, Compound 35 (2.01 g, 5.00 mmol) was added to 38 ml of DMF, followed by stirring, and phosphoryl bromide (3.58 g, 12.5 mmol) was added thereto little by little. The resultant was stirred for 1 hour at 100° C., thereby obtaining Compound 36 at a yield of 66%. In a nitrogen atmosphere, Compound 36 (1.42 g, 3.30 mmol) and Compound 2 (780 mg, 3.63 mmol) were added to 18 ml of acetic acid as a solvent, and the resultant was subjected to reflux for 3 hours. After being left to cool, the resultant was subjected to suction filtration and recrystallized over N,N-dimethylacetamide. By performing suction filtration, Compound D22 was obtained at a yield of 77%.

(Synthesis of Compound D23)

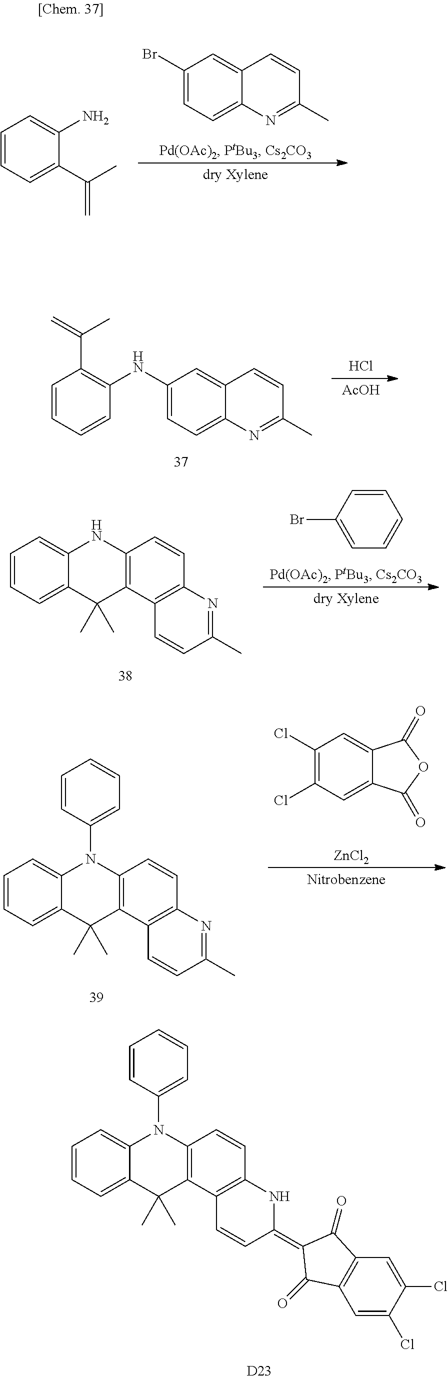

2-iso-propenylaniline (4.20 g, 31.5 mmol), palladium acetate (210 mg, 0.95 mmol), tri(t-butyl)phosphine (570 mg, 2.80 mmol), cesium carbonate (20.5 g, 62.9 mmol), and 6-bromo-2-methylquinoline (6.99 g, 31.5 mmol) were dissolved in 50 ml of xylene and reacted under reflux at a boiling point for 5 hours in a nitrogen atmosphere, thereby obtaining Compound 37 at a yield of 75%. Compound 37 (5.48 g, 20.0 mmol) was added to a mixed solvent consisting of 40 ml of acetic acid and 8 ml of hydrochloric acid, and the resultant was stirred for 30 minutes at 60° C., thereby obtaining Compound 38 at a yield of 80%. Compound 38 (2.7 g, 10.0 mmol), palladium acetate (224 g, 1.0 mmol), tri(t-butyl)phosphine (607 mg, 3.0 mmol), cesium acetate (6.51 g, 20.0 mmol), and bromobenzene (1.56 g, 10.0 mmol) were dissolved in 45 ml of xylene and reacted under reflux at a boiling point for 9 hours in a nitrogen atmosphere, thereby obtaining Compound 39 at a yield of 90%. Compound 39 (0.91 g, 2.6 mmol), 4,5-dichlorophthalic acid (1.12 g, 5.2 mmol), and zinc (II) chloride (0.40 g, 2.9 mmol) were added to 2 ml of nitrobenzene, and the resultant was heated and stirred for 9 hours at 200° C., thereby obtaining Compound 23 at a yield of 11%.

(Synthesis of Compound D24)

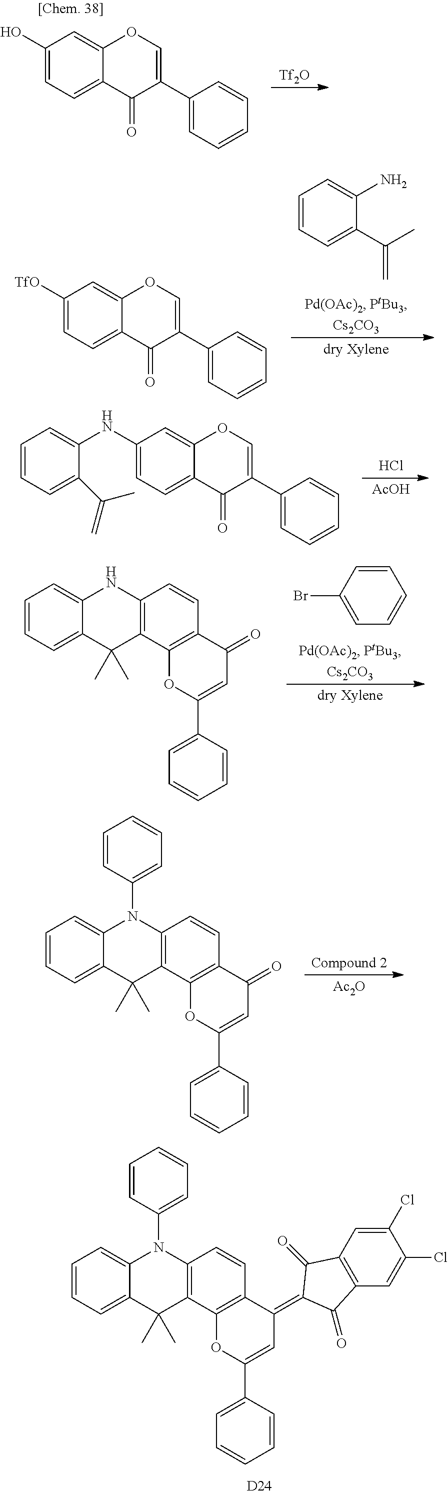

Compound D24 was synthesized according to the above scheme.

(Synthesis of Compound D25)

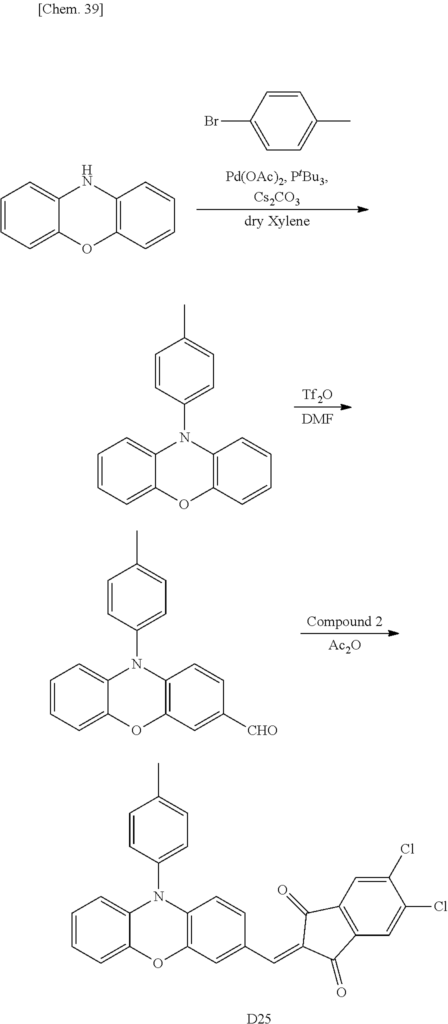

Compound D25 was synthesized according to the above scheme.

(Synthesis of Compound D26)

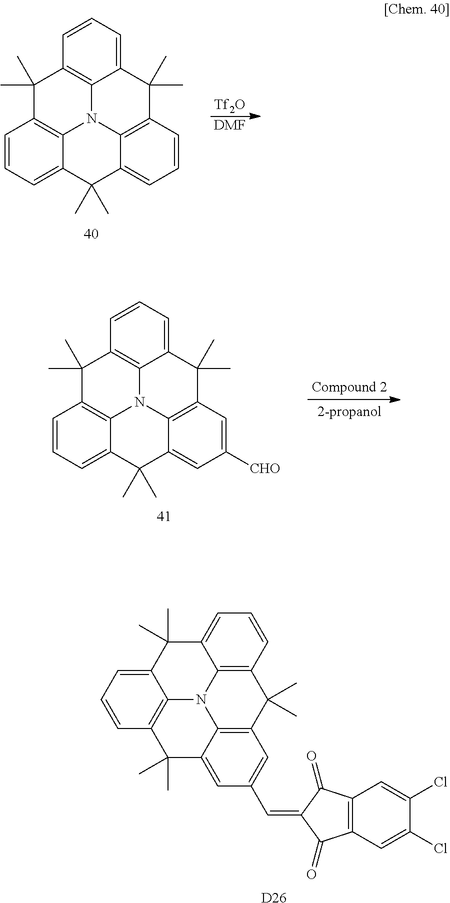

Compound 40 was synthesized according to the method described in Org. Lett. 2009, 11, 1-4. Compound 40 (400 mg, 1.09 mmol) was dissolved in dehydrated N,N-dimethylformamide (4 ml), and trifluoromethanesulfonic anhydride (0.3 ml) was added dropwise thereto. In a nitrogen atmosphere, the resultant was heated to 90° C. and stirred for 1 hour, thereby obtaining Compound 41 at a yield of 94%. In a nitrogen atmosphere, Compound 40 (400 mg, 1.02 mmol) and Compound 2 (241 mg, 1.12 mmol) were added to 2-propanol (7 ml) as a solvent, and the resultant was subjected to reflux for 3 hours. After being left to cool, the resultant was subjected to suction filtration and recrystallized over tetrahydrofuran. By performing suction filtration, Compound D26 was obtained at a yield of 52%.

(Synthesis of Compound D27)

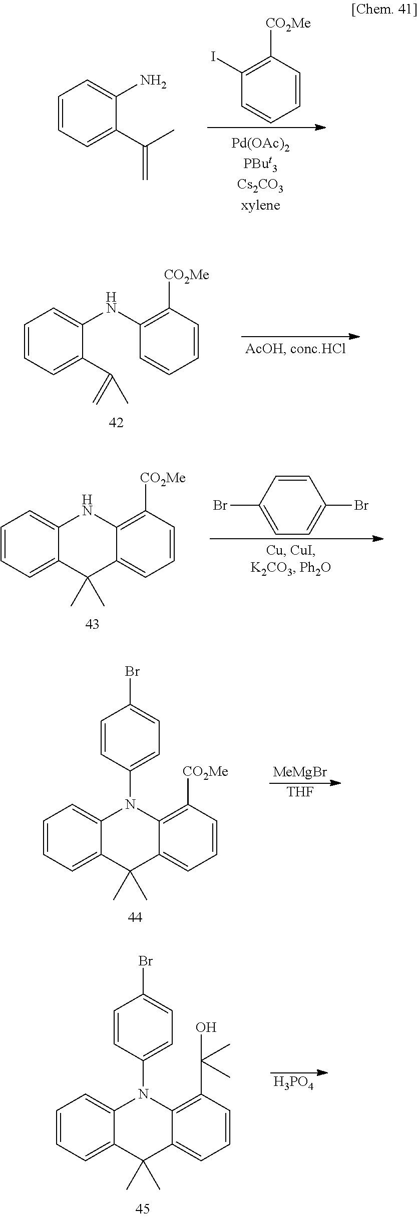

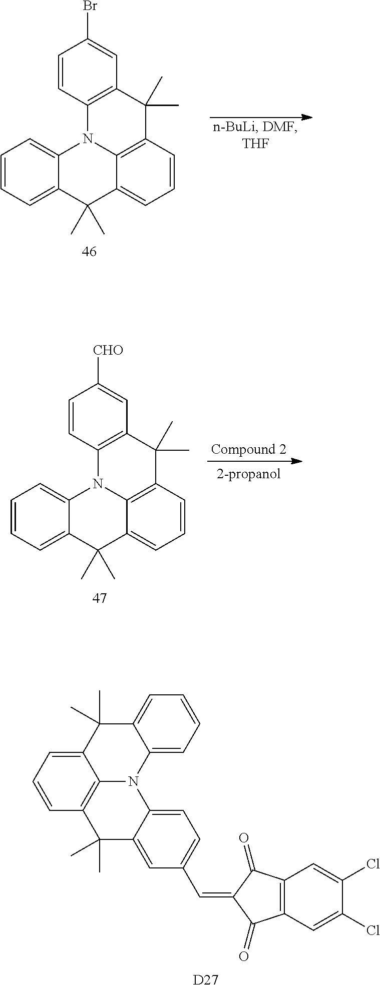

Isopropenylaniline, methyl o-idobenzoate, palladium acetate, tri(t-butyl)phosphine, and cesium carbonate were dissolved in 50 ml of xylene and reacted under reflux for 5 hours in a nitrogen atmosphere, thereby obtaining Compound 42 at a yield of 78%. Compound 42 was added to a mixed solvent consisting of acetic acid and concentrated hydrochloric acid, and the resultant was stirred for 1 hour at 60° C., thereby obtaining Compound 43 at a yield of 82%. Compound 43, p-dibromobenzene, copper powder, copper iodide, and potassium carbonate were added to diphenyl ether, and the resultant was subjected to reflux for 5 hours, thereby obtaining Compound 44 at a yield of 76%. Compound 44 was dissolved in dehydrated tetrahydrofuran, and to the resultant, a 3 M methyl Grignard reagent (ethyl ether solution) was added dropwise. Subsequently, the resultant was heated up to a reflux temperature and stirred for 1 hour, thereby obtaining Compound 45 at a yield of 95%. Compound 45 was added to phosphoric acid and stirred for 2 hours at 90° C., thereby obtaining Compound 46 at a yield of 45%. Compound 46 was dissolved in dehydrated tetrahydrofuran and cooled to −40° C. by using a dry ice bath. Thereafter, to the resultant, n-butyllithium (1.6 M, in Hexane) was added dropwise, followed by stirring for 15 minutes. To the resultant, dehydrated N,N-dimethylformamide was added dropwise, and the dry ice bath was removed. 1 M diluted hydrochloric acid was added to the resultant, thereby obtaining Compound 47 at a yield of 68%. In a nitrogen atmosphere, Compound 47 and Compound 2 were added to 2-propanol as a solvent, and the resultant was subjected to reflux for 3 hours. After being left to cool, the resultant was subjected to suction filtration and recrystallized over tetrahydrofuran. By performing suction filtration, Compound D27 was obtained.

(Synthesis of Compound D28)

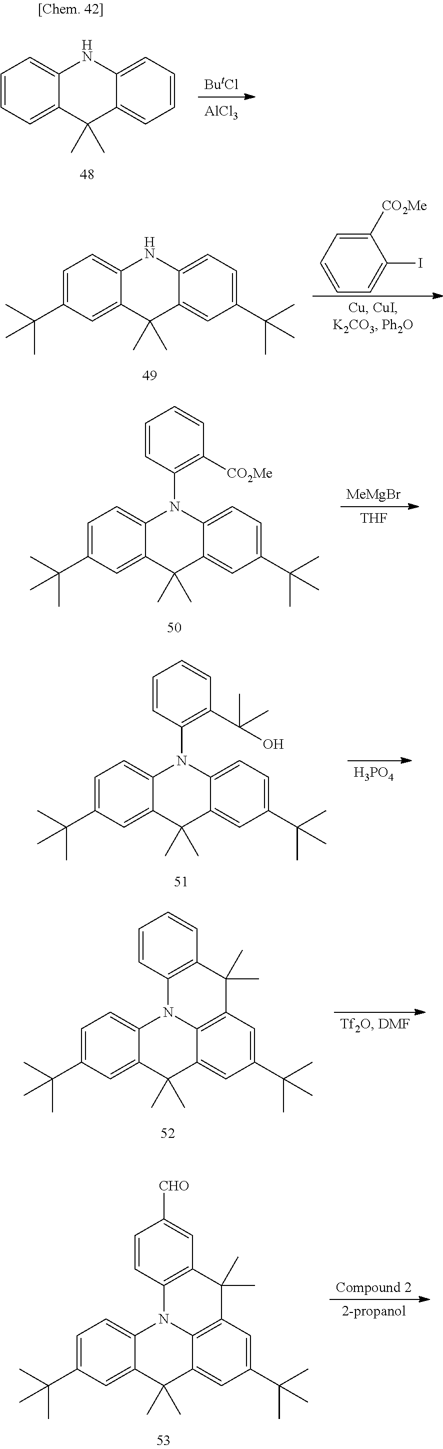

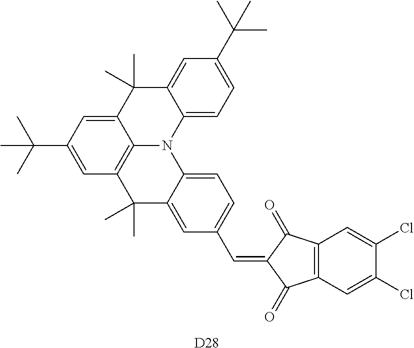 [SMILES: CC(C)(C)c1ccc2c(c1)C(C)(C)c1cc(C(C)(C)C)cc3c1N2c1ccc(C=C2C(=O)c4cc(Cl)c(Cl)cc4C2=O)cc1C3(C)C ; "D28"]

Compound 48 was synthesized according to the method described in Chemishe Berichte 1980, 113, 358-384. Compound 48 was dissolved in 1,2-dichloroethane and cooled in an ice bath, and then aluminum chloride and t-butylchloride were added thereto. The reaction solution was heated to 60° C. and stirred for 1 hour, thereby obtaining Compound 49 at a yield of 80%. Compound 49 and methyl o-iodobenzoate were dissolved in diphenylether, and copper powder, copper iodide, and potassium carbonate were added thereto. In a nitrogen atmosphere, the resultant was heated to 180° C. and stirred for 4 hours, thereby obtaining Compound 50 at a yield of 86%. Compound 50 was dissolved in dehydrated tetrahydrofuran, and to the resultant, a 3 M methyl Grignard reagent (ethyl ether solution) was added dropwise. Subsequently, the resultant was heated to a reflux temperature and stirred for 1 hour, thereby obtaining Compound 51 at a yield of 95%. Compound 51 was added to phosphoric acid and stirred for 2 hours at 90° C., thereby obtaining Compound 52 at a yield of 40%. Compound 52 was dissolved in dehydrated N,N-dimethylformamide, and trifluoromethanesulfonic anhydride was added dropwise thereto. In a nitrogen atmosphere, the resultant was heated to 90° C. and stirred for 1 hour, thereby obtaining Compound 53 at a yield of 80%. In a nitrogen atmosphere, Compound 52 and benzoindanedione were added to 2-propanol as a solvent, and the resultant was subjected reflux for 3 hours. After being left to cool, the resultant was subjected to suction filtration and recrystallized over tetrahydrofuran. By performing suction filtration, Compound D28 was obtained at a yield of 56%.

(Synthesis of Compound D29)

Compound D29 was synthesized according to the same procedure as the synthesis method of Compound D7, except that 4-bromo-t-butylbenzene was used instead of bromobenzene.

(Synthesis of Compound D30)

Compound D30 was synthesized according to the same procedure as the synthesis method of Compound D7, except that 3-bromo-t-butylbenzene was used instead of bromobenzene.

(Synthesis of Compound D31)

Compound D31 was synthesized according to the same procedure as the synthesis method of Compound D7, except that 4-bromotoluene was used instead of bromobenzene.

(Synthesis of Compound D32)

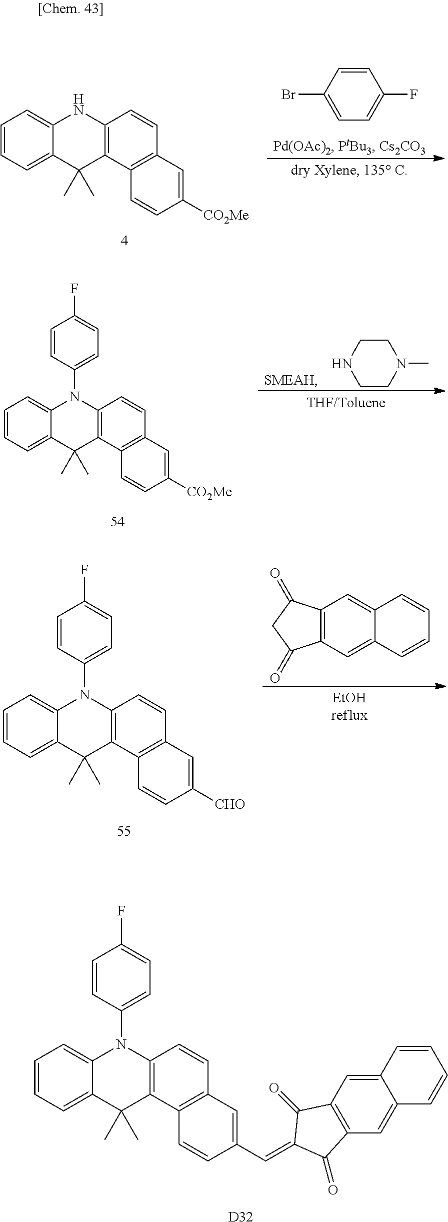

Compound 55 was synthesized according to the same procedure as the synthesis method of Compound D7, except that 4-bromofluorobenzene was used instead of bromobenzene. Moreover, Compound D32 was obtained at a yield of 61% according to the same procedure as the synthesis method of Compound D7, except that Compound 55 was used instead of Compound 19, and benzoindanedione was used instead of Compound 2.

(Synthesis of Compound D33)

Compound D33 was obtained at a yield of 54% according to the same procedure as the synthesis method of Compound D32, except that Compound 2 was used instead of benzoindanedione.

(Synthesis of Compound D34)

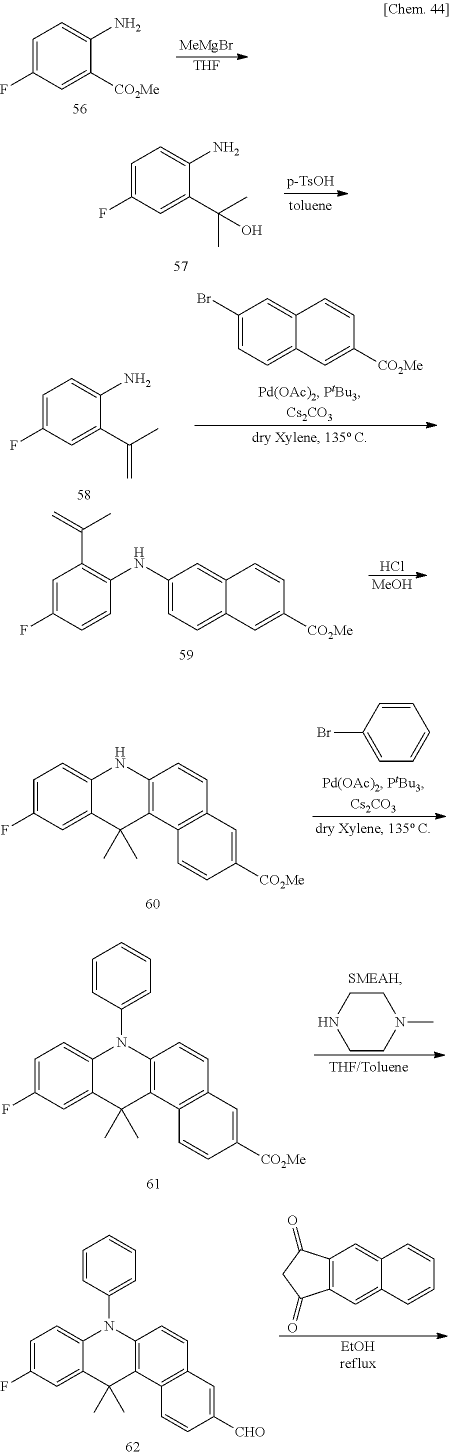

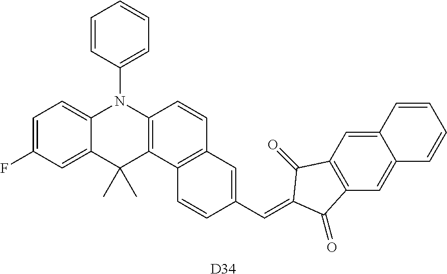 [SMILES: CC1(C)c2cc(F)ccc2N(c2ccccc2)c2ccc3cc(C=C4C(=O)c5cc6ccccc6cc5C4=O)ccc3c21 ; "D34"]

Compound 56 (25 g, 148 mmol) was dissolved in 300 mL of dehydrated tetrahydrofuran, and the resultant was cooled to 0° C. After a 1 M methyl magnesium bromide tetrahydrofuran solution (600 mL, 600 mmol) was added dropwise thereto, the reaction solution was heated to room temperature. Thereafter, the resultant was quenched with ice and subjected to extraction by using ethyl acetate. After being concentrated, the resultant was purified by silica gel column chromatography, thereby obtaining Compound 57 at a yield of 93%. Compound 57 (23.3 g, 138 mmol) and p-toluenesulfonic acid monohydrate (2.6 g, 13.7 mmol) were added to 690 mL of toluene, and the resultant was heated under reflux. The reaction solution was concentrated and purified by silica gel column chromatography, thereby obtaining Compound 58 at a yield of 90%. Synthesis of Compound 59 to Compound D34 were performed according to the procedure of the synthesis method of compound D1, and in this manner, Compound D34 was obtained.

(Synthesis of Compound D35)

Compound D35 was synthesized according to the following scheme.

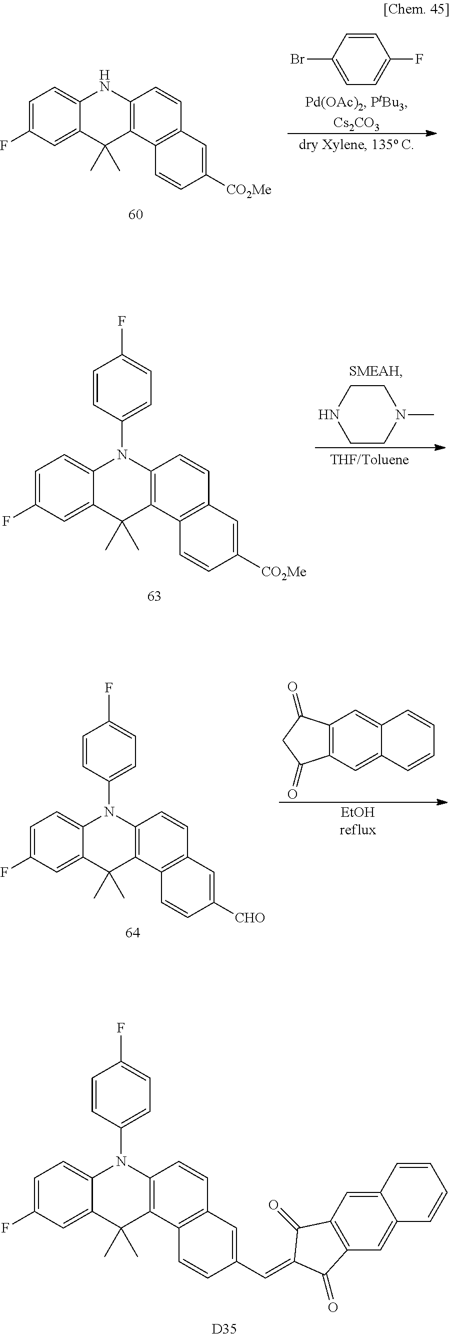

Compound D36 was synthesized according to the following scheme.

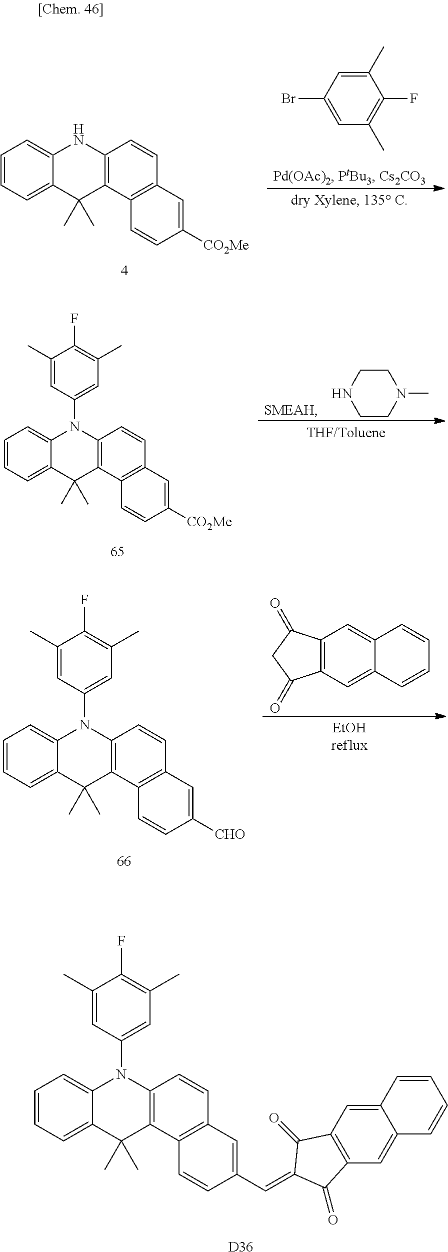

(Synthesis of Compound D37)

Compound D37 was synthesized according to the following scheme.

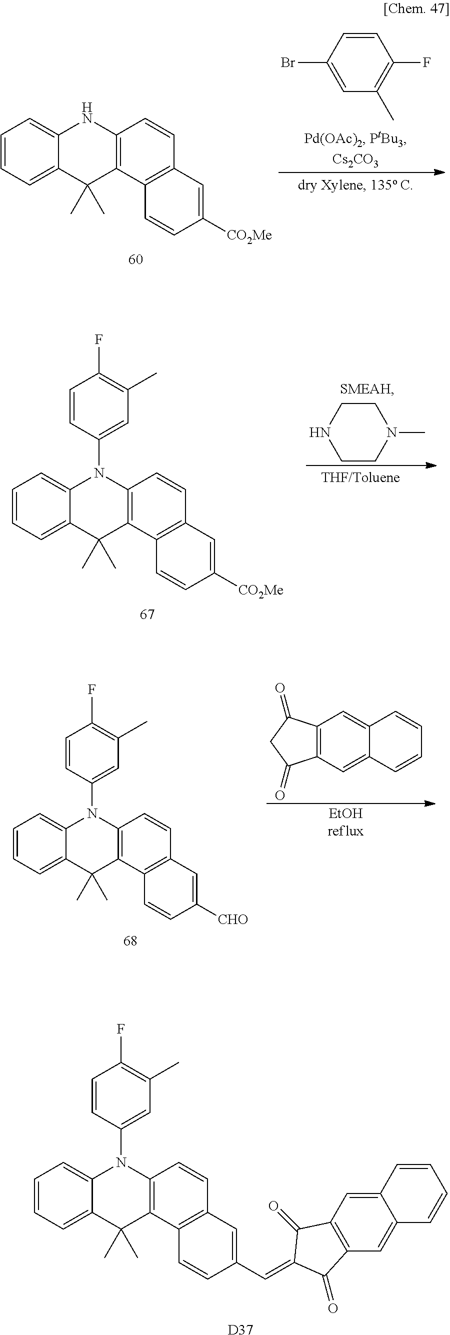

(Synthesis of Compound D38)

Compound D38 was synthesized according to the following scheme.

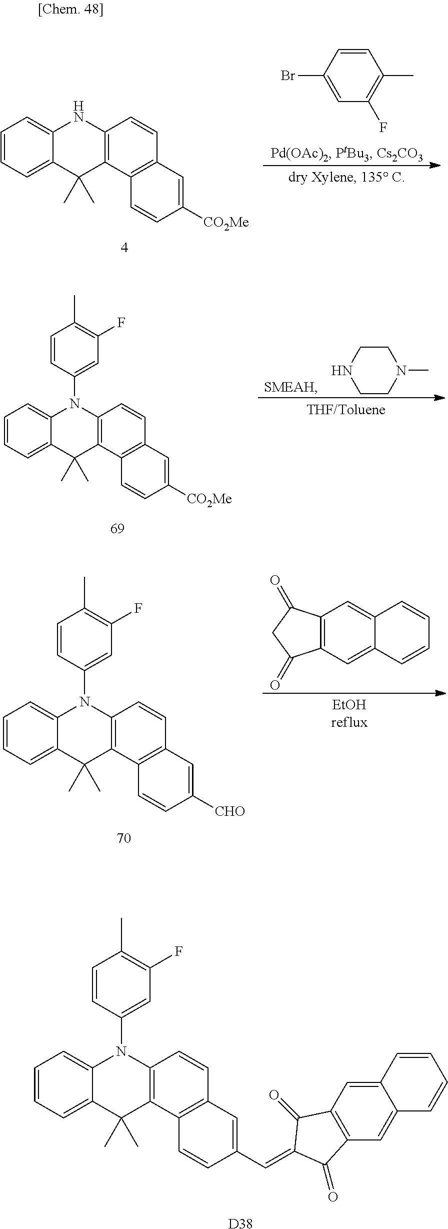

(Synthesis of Compound D39)

Compound D39 was synthesized according to the following scheme.

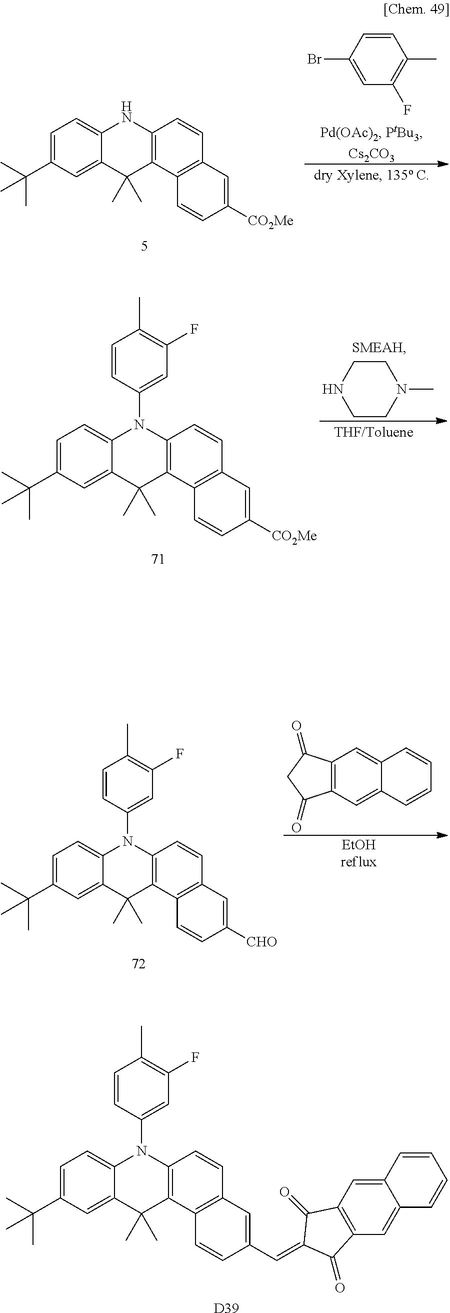

(Synthesis of Compound D40)

Compound D40 was synthesized according to the following scheme.

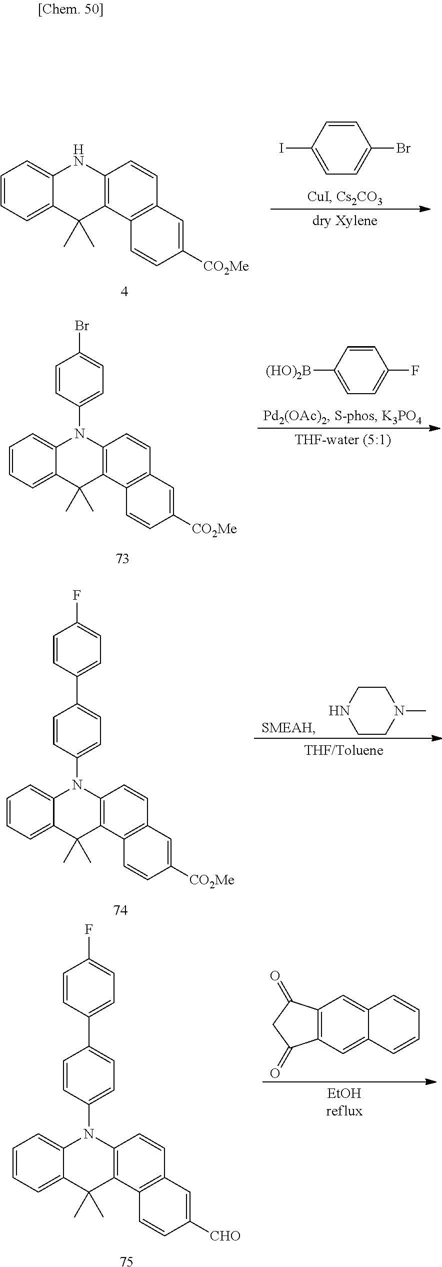

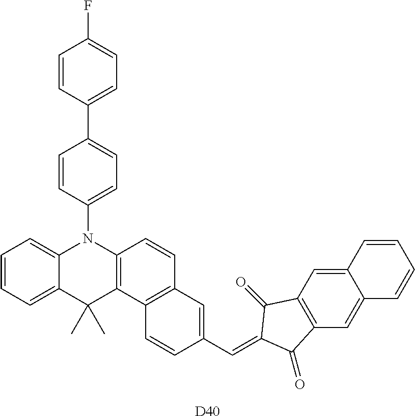 [SMILES: CC1(C)c2ccccc2N(c2ccc(-c3ccc(F)cc3)cc2)c2ccc3cc(C=C4C(=O)c5cc6ccccc6cc5C4=O)ccc3c21 ; "D40"]

Compound 4 (5 g, 15.8 mmol), l-bromo-4-iodobenzene (13.7 g, 47.2 mmol), copper iodide (1.5 g, 7.88 mmol), and cesium carbonate (10.3 g, 31.5 mmol) were added to 6 mL of dehydrated xylene, and the resultant was heated under reflux for 12 hours. The reaction solution was cooled to room temperature and subjected to extraction by using toluene.

After being concentrated, the reaction solution was purified by silica gel column chromatography, thereby obtaining Compound 73 at a yield of 90%. Compound 73 (1.04 g, 2.20 mmol), S-phos(2-dicyclohexylphosphino-2′,6′-dimethoxybiphenyl) (107 mg, 0.26 mmol), and potassium phosphate (1.87 g, 8.80 mmol) were dissolved in 20 mL of tetrahydrofuran and 4 mL of water, and then the resultant was deaerated under reduced pressure. To the resultant, 4-fluorophenylboronic acid (462 mg, 3.30 mmol) and palladium acetate (25 mg, 0.11 mmol) were added, and the resultant was heated under reflux for 2 hours. Extraction was performed on the reaction solution, and the resultant was concentrated and purified by silica gel column chromatography, thereby obtaining Compound 74 at a yield of 95%.

By using the obtained Compound 74, Compound D40 was obtained according to the above scheme (procedure at the time of producing Compound 7 and Compound D1 in the synthesis method of Compound D1).

(Synthesis of Compounds C1 to C14)

Comparative Compounds C1 to C13 were synthesized according to the description of EP2 292 586 A2 and US 2005/0065451 A1, except that an acidic nucleus was appropriately changed to Compound 11 and Compound 17 described above.

Comparative Compound C14 was synthesized according to the method described in Chemistry of Heterocyclic Compounds (New York, N.Y., United States), 1988, p. 611-616, except that an acidic nucleus was changed to Compound 2.

<Method for Measuring Melting Point (Tm)>

A melting point (Tm) of the above compounds was measured by differential scanning calorimetry (TG/DTA 6200 AST-2 manufactured by SII NanoTechnology Inc.) by being defined as an endothermic peak top observed when the temperature is increased at a scanning speed of 10 K/min. Tm of each compound is shown in the column of “Tm” of Table 1.

<Method for Measuring Vapor Deposition Temperature (Ts)>

A vapor deposition temperature of the compounds described above was measured by being defined as a temperature in a steady state at which a vapour deposition speed reaches 0.4 Å/s (0.4×10⁻¹⁰ m/s) when a pot containing the compound is heated at a degree of vacuum of 1.0×10⁻⁴ Pa. Ts of each compound is shown in the column of “Ts” of Table 1.

In addition, in Table 1, “ΔT” shows a difference between Ts and Tm. The greater the value of “ΔT”, the further the degradation of the compound is suppressed during vapor deposition.

<Continuous Vapor Deposition Test>

Vapor deposition characteristics of the compound were evaluated by continuously forming a film of the compound at each vapor deposition temperature and performing HPLC to measure purity of the vapor-deposited film at the time when 5 hours elapsed from the formation of film. If decrease in purity of the vapor-deposited film at the time when 5 hours elapsed from the formation of film is less than 1%, the film is classified into “A”; if it is equal to or greater than 1% but less than 5%, the film is classified into “B”; if it is equal to or greater than 5% but less than 10%, the film is classified into “C”; and if it is equal to or greater than 10%, the film is classified into “D”. The results are shown in the column of “Vapor deposition characteristics” of Table 1. For practical use, the film is desirably classified into “A” or “B”, and particularly desirably classified into “A”.

<Classification According to Sensitivity to Red>

In order that the present photoelectric conversion element has spectral sensitivity in the entire visible light region, it is particularly important for absorption of the compound to widen up to a red light region of a long wavelength side.

Therefore, if a maximum absorption wavelength λmax of the compound is 560 nm or longer, the compound is classified into “A”; if it is less than 560 nm but equal to or longer than 540 nm, the compound is classified into “B”; if it is less than 540 nm but equal to or longer than 520 nm, the compound is classified into “C”; and if it is less than 520 nm, the compound is classified into “D”. The results are shown in the column of “R sensitivity” of Table 1. For practical use, the compound is desirably classified into “A” or “B”.

<Preparation of Photoelectric Conversion Element>

The photoelectric conversion element in the form shown in FIG. 1A was prepared. Herein, the photoelectric conversion element is configured with the lower electrode 11, the electron blocking layer 16A, the photoelectric conversion layer 12, and the upper electrode 15.

Specifically, on a glass substrate, amorphous ITO is formed into a film by a sputtering method, thereby forming the lower electrode 11 (thickness: 30 nm). Thereafter, on the lower electrode 11, the following compound (EB-1) was formed into a film by a vacuum heating vapor deposition method, thereby forming the electron blocking layer 16A (thickness: 100 nm). Moreover, in a state where the substrate temperature was being controlled to be 25° C., on the electron blocking layer 16A, the compound (D1 to D40 and C1 to C14) and fullerene C₆₀ were co-deposited and formed into a film by vacuum heating vapor deposition such that the thickness thereof became 100 nm and 300 nm respectively in terms of a single layer, thereby forming the photoelectric conversion layer 12. Furthermore, on the photoelectric conversion layer 12, amorphous ITO was formed into a film by a sputtering method, thereby forming the upper electrode 15 (transparent conductive film) (thickness: 10 nm). On the upper electrode 15, a SiO film was formed as a sealing layer by heating vapor deposition, and on this film, an aluminum oxide (Al₂O₃) layer was formed by an ALCVD method, thereby preparing the photoelectric conversion element.

In the photoelectric conversion layer 12, a ratio of the content of fullerene C₆₀ (a film thickness expressed in terms of a single layer of fullerene C₆₀/(a film thickness expressed in terms of a single layer of the compound+a film thickness expressed in terms of a single layer of fullerene C₆₀)) was 75% by volume.

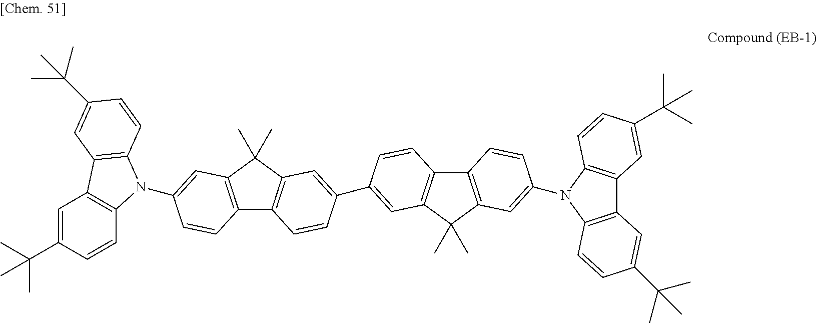 [SMILES: CC(C)(C)c1ccc2c(c1)c1cc(C(C)(C)C)ccc1n2-c1ccc2c(c1)C(C)(C)c1cc(-c3ccc4c(c3)C(C)(C)c3cc(-n5c6ccc(C(C)(C)C)cc6c6cc(C(C)(C)C)ccc65)ccc3-4)ccc1-2]

<Checking Driving of Element (Measuring Dark Current)>

Each of the obtained elements was checked so as to confirm whether the element functions as a photoelectric conversion element.

To the lower electrode and the upper electrode of each of the elements (Examples 1 to 40 and Comparative examples 1 to 14), voltage was applied such that an electric field intensity became 2.5×10⁵ V/cm. As a result, in any of the elements, dark currents of equal to or weaker than 100 nA/cm² were observed in a dark place, and electric currents of equal to or stronger than 10 μA/cm² were observed in a bright place. Therefore, the photoelectric conversion element was confirmed to function.

<Heat Resistance of Element>

Each of the obtained elements was measured in terms of the change in external quantum efficiency and dark currents before and after thermal annealing.

For the lower electrode and the upper electrode of each of the obtained elements shown in FIG. 1, a driving voltage (about 10 V) was set such that electric currents at a dark place in the element of Comparative example 1 became 1 nA/cm₂, and the element was driven at the voltage. At this time, the external quantum efficiency and dark currents before and after thermal annealing were measured.

The thermal annealing was performed by holding the element on a hot plate of 220° C. for 30 minutes in a nitrogen atmosphere. After the temperature was returned to room temperature, the dark currents and external quantum efficiency were measured.

From the external quantum efficiency before and after the thermal annealing treatment, a rate of decrease in the external quantum efficiency caused by the thermal annealing treatment (=(external quantum efficiency before thermal annealing treatment−external quantum efficiency after thermal annealing treatment)/external quantum efficiency before thermal annealing treatment×100(%)) was obtained.

Moreover, from the level of dark currents before and after the thermal annealing treatment, a rate of increase in dark currents caused by the thermal annealing treatment (=(level of dark currents after thermal annealing treatment−level of dark currents before thermal annealing treatment)/level of dark currents before thermal annealing treatment×100(%)) was obtained.

If both the rate of decrease in external quantum efficiency and the rate of increase in dark currents were less than 3% in an element, the element was evaluated to be “OK”. If one of them deteriorated to a degree equal to or higher than 3%, the element was evaluated to be “NG”. The results are shown in the column of “Heat resistance at 220° C.” in Table 1.

<Evaluation of Response Speed (90% Signal Rise Time)>

An electric field of 2×10⁵V/cm was applied to the obtained photoelectric conversion element, and at this time, a relative response speed (a rise time taken for obtaining 90% signal intensity from 0) was measured. Provided that the rise time of Example 1 was 1, if the rise time of an element was less than 1.0 relative to Example 1, the element was classified into “AA”; if it was equal to or greater than 1.0 but less than 1.5, the element was classified into “A”; if it was equal to or greater than 1.5 but less than 2, the element was classified into “B”; if it was equal to or greater than 2 but less than 3, the element was classified into “C”; and if it was equal to or greater than 3, the element was classified into “D”. The results are shown in the column of “Response” of Table 1. For measuring photoelectric conversion performance of each element, light was caused to enter the element from the upper electrode (transparent conductive film). For practical use, it is desirable for the elements to be classified into “AA”, “A”, or “B”.

(Relative value when Example 1 is regarded as 1)=[rise time taken for obtaining 90% signal intensity from 0 in Example (or Comparative example) X]/[rise time taken for obtaining 90% signal intensity from 0 in Example 1]

<Evaluation of Efficiency (External Quantum Efficiency)_{>}

Voltage was applied to the obtained photoelectric conversion element such that an electric field intensity became 2.0×10⁵ V/cm, and external quantum efficiency at a maximum sensitivity wavelength at the voltage was measured. Provided that the external quantum efficiency of Example 1 is 1, if the external quantum efficiency of an element was equal to or greater than 0.9 relative to Example, the element was classified into “A”; if it was equal to or greater than 0.8 but less than 0.9, the element was classified into “B”; if it was equal to or greater than 0.7 but less than 0.8, the element was classified into “C”; and if it was less than 0.7, the element was classified into “D”. The results are shown in the column of “Efficiency” of Table 1. It is preferable for the element to be classified into “A” or “B”.

TABLE 1

| | | | | | | | | | Vapor | | | | |
|---|---|---|---|---|---|---|---|---|---|---|---|---|---|
| | | | | | | | | | depo- | | Heat | | |
| | | | | | | | | | sition | | resistance | | |
| | | | Acceptor | | Δmax/ | Ts/ | Tm/ | ΔT/ | charac- | R | at | Re- | |
| | No. | Donor size | site | Mw | nm | ° C. | ° C. | ° C. | teristics | sensitivity | 220° C. | sponse | Efficiency |
| Example 1 | D1 | 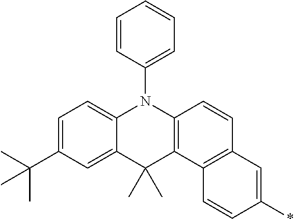 [SMILES: *c1ccc2c3c(ccc2c1)N(c1ccccc1)c1ccc(C(C)(C)C)cc1C3(C)C] | 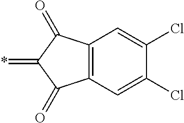 [SMILES: *=C1C(=O)c2cc(Cl)c(Cl)cc2C1=O] | 617 | 572 | 249 | 367 | 118 | A | A | OK | A | A |
| Example 2 | D2 | ″ | 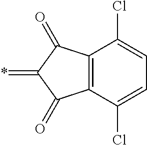 [SMILES: *=C1C(=O)c2c(Cl)ccc(Cl)c2C1=O] | 617 | 571 | 258 | 355 | 97 | A | A | OK | A | A |
| Example 3 | D3 | ″ | 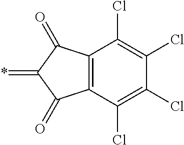 [SMILES: *=C1C(=O)c2c(Cl)c(Cl)c(Cl)c(Cl)c2C1=O] | 685 | 591 | 260 | 342 | 82 | B | A | OK | A | B |
| Example 4 | D4 | ″ | 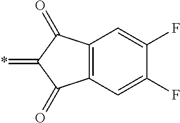 [SMILES: *=C1C(=O)c2cc(F)c(F)cc2C1=O] | 584 | 561 | 231 | 355 | 124 | A | A | OK | B | B |
| Example 5 | D5 | ″ | 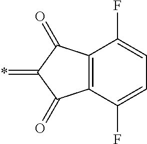 [SMILES: *=C1C(=O)c2c(F)ccc(F)c2C1=O] | 584 | 567 | 227 | 302 | 75 | B | A | OK | A | B |
| Example 6 | D6 | ″ | 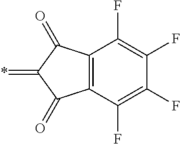 [SMILES: *=C1C(=O)c2c(F)c(F)c(F)c(F)c2C1=O] | 620 | 581 | 230 | 378 | 148 | A | A | OK | C | C |
| Example 7 | D7 | 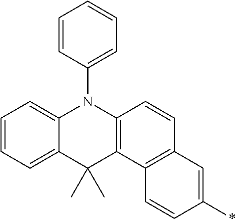 [SMILES: *c1ccc2c3c(ccc2c1)N(c1ccccc1)c1ccccc1C3(C)C] | 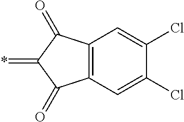 [SMILES: *=C1C(=O)c2cc(Cl)c(Cl)cc2C1=O] | 560 | 560 | 234 | 320 | 86 | A | A | OK | A | A |
| Example 8 | D8 | ″ | 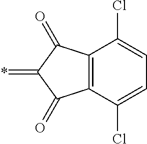 [SMILES: *=C1C(=O)c2c(Cl)ccc(Cl)c2C1=O] | 560 | 560 | 246 | 324 | 78 | B | A | OK | AA | A |
| Example 9 | D9 | ″ | 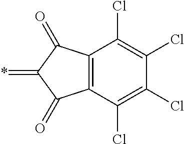 [SMILES: *=C1C(=O)c2c(Cl)c(Cl)c(Cl)c(Cl)c2C1=O] | 629 | 580 | 261 | 347 | 86 | B | A | OK | AA | A |
| Example 10 | D10 | ″ | 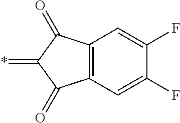 [SMILES: *=C1C(=O)c2cc(F)c(F)cc2C1=O] | 528 | 547 | 229 | 331 | 102 | A | B | OK | B | B |
| Example 11 | D11 | ″ | 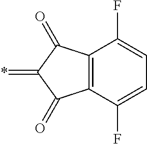 [SMILES: *=C1C(=O)c2c(F)ccc(F)c2C1=O] | 528 | 553 | 219 | 266 | 47 | C | B | OK | B | B |
| Example 12 | D12 | ″ | 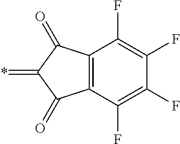 [SMILES: *=C1C(=O)c2c(F)c(F)c(F)c(F)c2C1=O] | 564 | 569 | 219 | 341 | 122 | A | A | OK | C | C |
| Example 13 | D13 | ″ | 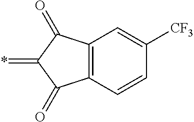 [SMILES: *=C1C(=O)c2ccc(C(F)(F)F)cc2C1=O] | 560 | 558 | 202 | 255 | 53 | C | B | OK | C | C |
| Example 14 | D14 | ″ | 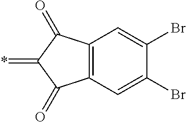 [SMILES: *=C1C(=O)c2cc(Br)c(Br)cc2C1=O] | 649 | 570 | 276 | 353 | 77 | C | A | OK | A | B |
| Example 15 | D15 | ″ | 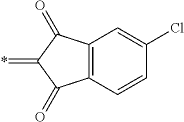 [SMILES: *=C1C(=O)c2ccc(Cl)cc2C1=O] | 526 | 547 | 218 | 273 | 55 | C | B | OK | A | A |
| Example 16 | D16 | ″ | 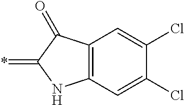 [SMILES: *=C1Nc2cc(Cl)c(Cl)cc2C1=O] | 547 | 558 | 212 | 308 | 96 | B | B | OK | B | C |
| Example 17 | D17 | 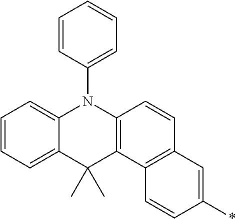 [SMILES: *c1ccc2c3c(ccc2c1)N(c1ccccc1)c1ccccc1C3(C)C] | 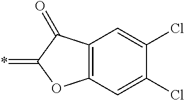 [SMILES: *=C1Oc2cc(Cl)c(Cl)cc2C1=O] | 548 | 520 | 210 | 293 | 83 | B | C | OK | A | B |
| Example 18 | D18 | ″ | 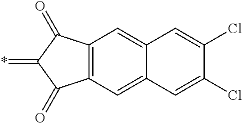 [SMILES: *=C1C(=O)c2cc3cc(Cl)c(Cl)cc3cc2C1=O] | 611 | 591 | 290 | 352 | 62 | C | A | OK | B | A |
| Example 19 | D19 | 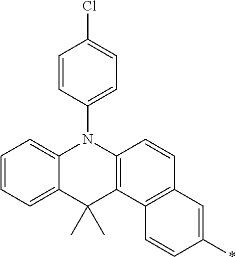 [SMILES: *c1ccc2c3c(ccc2c1)N(c1ccc(Cl)cc1)c1ccccc1C3(C)C] | 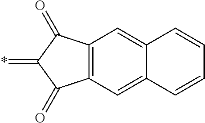 [SMILES: *=C1C(=O)c2cc3ccccc3cc2C1=O] | 576 | 553 | 262 | 362 | 100 | A | B | OK | A | B |
| Example 20 | D20 | 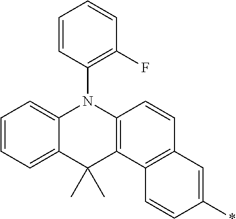 [SMILES: *c1ccc2c3c(ccc2c1)N(c1ccccc1F)c1ccccc1C3(C)C] | 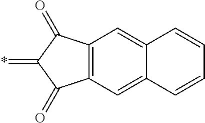 [SMILES: *=C1C(=O)c2cc3ccccc3cc2C1=O] | 560 | 559 | 221 | 326 | 105 | A | B | OK | AA | A |
| Example 21 | D21 | 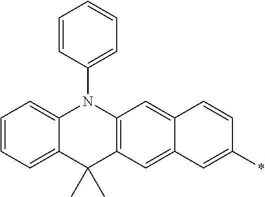 [SMILES: *c1ccc2cc3c(cc2c1)C(C)(C)c1ccccc1N3c1ccccc1] | 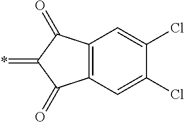 [SMILES: *=C1C(=O)c2cc(Cl)c(Cl)cc2C1=O] | 560 | 541 | 221 | 280 | 59 | C | B | OK | A | A |
| Example 22 | D22 | 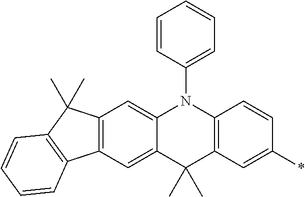 [SMILES: *c1ccc2c(c1)C(C)(C)c1cc3c(cc1N2c1ccccc1)C(C)(C)c1ccccc1-3] | 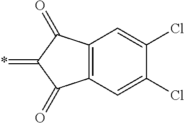 [SMILES: *=C1C(=O)c2cc(Cl)c(Cl)cc2C1=O] | 627 | 547 | 253 | 365 | 112 | A | B | OK | A | B |
| Example 23 | D23 | 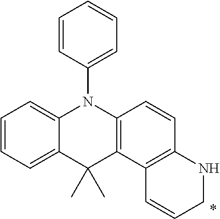 | 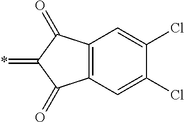 [SMILES: *=C1C(=O)c2cc(Cl)c(Cl)cc2C1=O] | 549 | 531 | 215 | 278 | 63 | B | C | OK | B | B |
| Example 24 | D24 | 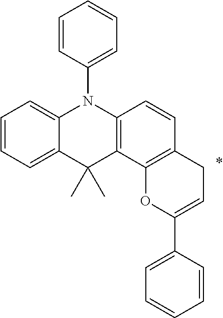 | 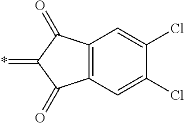 [SMILES: *=C1C(=O)c2cc(Cl)c(Cl)cc2C1=O] | 627 | 553 | 241 | 309 | 68 | A | B | OK | C | C |
| Example 25 | D25 | 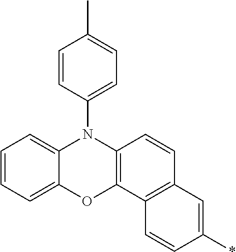 [SMILES: *c1ccc2c3c(ccc2c1)N(c1ccc(C)cc1)c1ccccc1O3] | 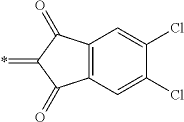 [SMILES: *=C1C(=O)c2cc(Cl)c(Cl)cc2C1=O] | 627 | 575 | 235 | 342 | 107 | A | A | OK | C | C |
| Example 26 | D26 | 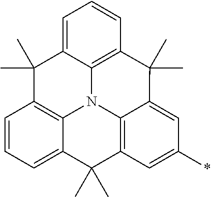 [SMILES: *c1cc2c3c(c1)C(C)(C)c1cccc4c1N3c1c(cccc1C2(C)C)C4(C)C] | 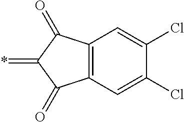 [SMILES: *=C1C(=O)c2cc(Cl)c(Cl)cc2C1=O] | 591 | 565 | 214 | 285 | 71 | B | A | OK | A | B |
| Example 27 | D27 | 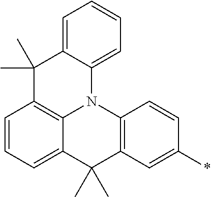 [SMILES: *c1ccc2c(c1)C(C)(C)c1cccc3c1N2c1ccccc1C3(C)C] | 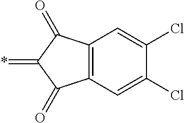 [SMILES: *=C1C(=O)c2cc(Cl)c(Cl)cc2C1=O] | 550 | 534 | 210 | 287 | 77 | B | C | OK | B | B |
| Example 28 | D28 | 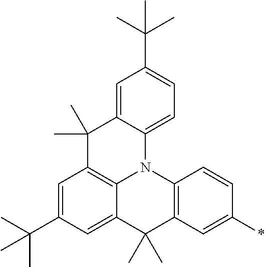 [SMILES: *c1ccc2c(c1)C(C)(C)c1cc(C(C)(C)C)cc3c1N2c1ccc(C(C)(C)C)cc1C3(C)C] | 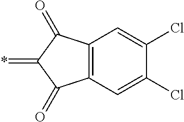 [SMILES: *=C1C(=O)c2cc(Cl)c(Cl)cc2C1=O] | 663 | 557 | 212 | 305 | 93 | A | B | OK | B | C |
| Example 29 | D29 | 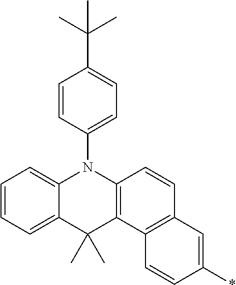 [SMILES: *c1ccc2c3c(ccc2c1)N(c1ccc(C(C)(C)C)cc1)c1ccccc1C3(C)C] | 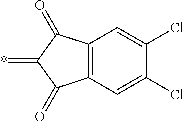 [SMILES: *=C1C(=O)c2cc(Cl)c(Cl)cc2C1=O] | 617 | 565 | 248 | 332 | 84 | B | A | OK | A | A |
| Example 30 | D30 | 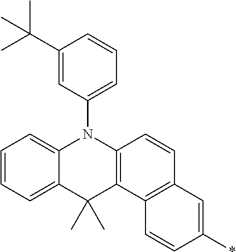 [SMILES: *c1ccc2c3c(ccc2c1)N(c1cccc(C(C)(C)C)c1)c1ccccc1C3(C)C] | 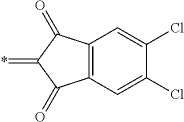 [SMILES: *=C1C(=O)c2cc(Cl)c(Cl)cc2C1=O] | 617 | 563 | 220 | 273 | 53 | C | A | OK | A | A |
| Example 31 | D31 | 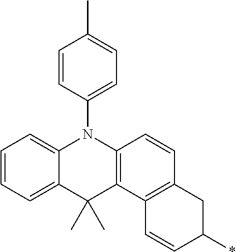 [SMILES: *C1C=Cc2c(ccc3c2C(C)(C)c2ccccc2N3c2ccc(C)cc2)C1] | 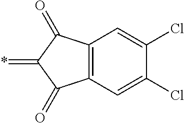 [SMILES: *=C1C(=O)c2cc(Cl)c(Cl)cc2C1=O] | 575 | 564 | 237 | 300 | 63 | B | A | OK | A | A |
| Example 32 | D32 | 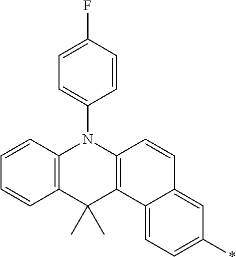 [SMILES: *c1ccc2c3c(ccc2c1)N(c1ccc(F)cc1)c1ccccc1C3(C)C] | 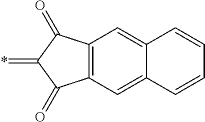 [SMILES: *=C1C(=O)c2cc3ccccc3cc2C1=O] | 560 | 557 | 241 | 335 | 94 | A | B | OK | AA | A |
| Example 33 | D33 | ″ | 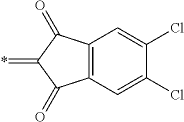 [SMILES: *=C1C(=O)c2cc(Cl)c(Cl)cc2C1=O] | 578 | 554 | 216 | 310 | 94 | A | B | OK | AA | B |
| Example 34 | D34 | 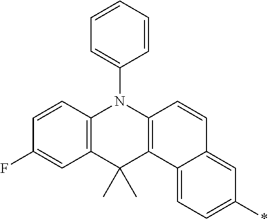 [SMILES: *c1ccc2c3c(ccc2c1)N(c1ccccc1)c1ccc(F)cc1C3(C)C] | 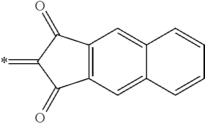 [SMILES: *=C1C(=O)c2cc3ccccc3cc2C1=O] | 560 | 561 | 240 | 336 | 96 | A | A | OK | AA | A |
| Example 35 | D35 | 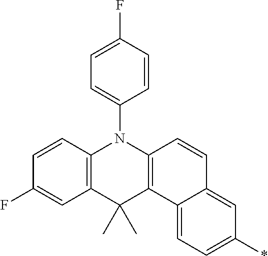 [SMILES: *c1ccc2c3c(ccc2c1)N(c1ccc(F)cc1)c1ccc(F)cc1C3(C)C] | 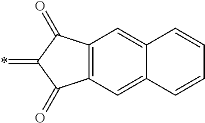 [SMILES: *=C1C(=O)c2cc3ccccc3cc2C1=O] | 578 | 554 | 240 | 353 | 113 | A | B | OK | A | B |
| Example 36 | D36 | 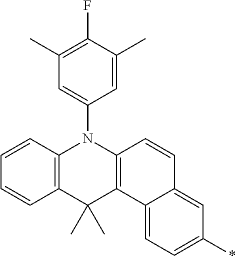 [SMILES: *c1ccc2c3c(ccc2c1)N(c1cc(C)c(F)c(C)c1)c1ccccc1C3(C)C] | 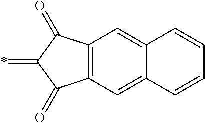 [SMILES: *=C1C(=O)c2cc3ccccc3cc2C1=O] | 588 | 565 | 231 | 310 | 79 | B | A | OK | AA | A |
| Example 37 | D37 | 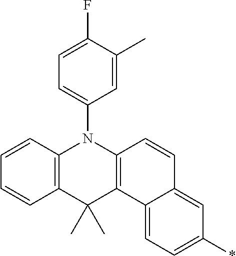 [SMILES: *c1ccc2c3c(ccc2c1)N(c1ccc(F)c(C)c1)c1ccccc1C3(C)C] | 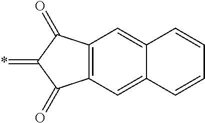 [SMILES: *=C1C(=O)c2cc3ccccc3cc2C1=O] | 574 | 560 | 235 | 304 | 69 | B | A | OK | AA | A |
| Example 38 | D38 | 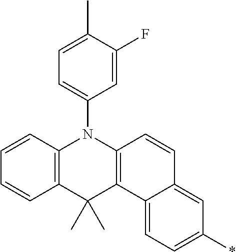 [SMILES: *c1ccc2c3c(ccc2c1)N(c1ccc(C)c(F)c1)c1ccccc1C3(C)C] | 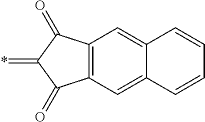 [SMILES: *=C1C(=O)c2cc3ccccc3cc2C1=O] | 574 | 559 | 243 | 332 | 89 | A | B | OK | AA | A |
| Example 39 | D39 | 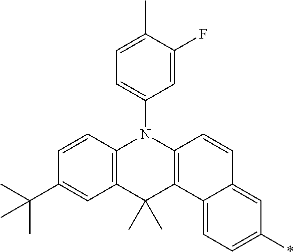 [SMILES: *c1ccc2c3c(ccc2c1)N(c1ccc(C)c(F)c1)c1ccc(C(C)(C)C)cc1C3(C)C] | 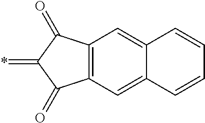 [SMILES: *=C1C(=O)c2cc3ccccc3cc2C1=O] | 630 | 572 | 251 | 333 | 82 | B | A | OK | A | A |
| Example 40 | D40 | 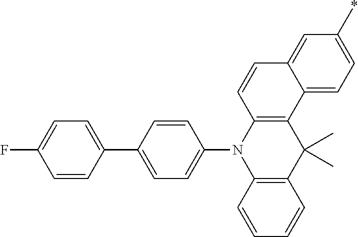 [SMILES: *c1ccc2c3c(ccc2c1)N(c1ccc(-c2ccc(F)cc2)cc1)c1ccccc1C3(C)C] | 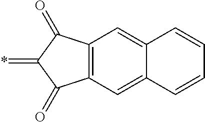 [SMILES: *=C1C(=O)c2cc3ccccc3cc2C1=O] | 636 | 564 | 253 | 359 | 106 | A | A | OK | AA | A |
| Comparative Example 1 | C1 | 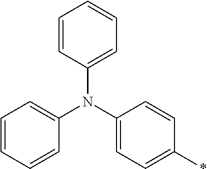 [SMILES: *c1ccc(N(c2ccccc2)c2ccccc2)cc1] | 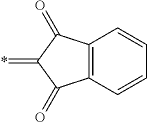 [SMILES: *=C1C(=O)c2ccccc2C1=O] | 401 | 492 | 197 | 251 | 54 | A | D | NG | C | D |
| Comparative Example 2 | C2 | ″ | 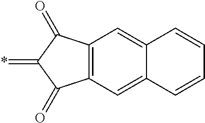 [SMILES: *=C1C(=O)c2cc3ccccc3cc2C1=O] | 452 | 519 | 211 | 271 | 60 | B | D | NG | AA | C |
| Comparative Example 3 | C3 | ″ | 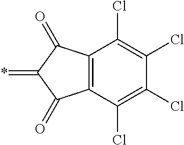 [SMILES: *=C1C(=O)c2c(Cl)c(Cl)c(Cl)c(Cl)c2C1=O] | 539 | 534 | 230 | 306 | 76 | B | C | NG | C | D |
| Comparative Example 4 | C4 | ″ | 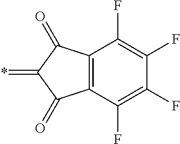 [SMILES: *=C1C(=O)c2c(F)c(F)c(F)c(F)c2C1=O] | 473 | 524 | 214 | 304 | 90 | A | C | NG | D | D |
| Comparative Example 5 | C5 | 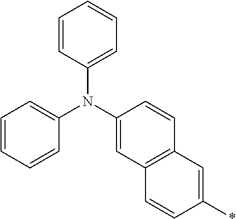 [SMILES: *c1ccc2cc(N(c3ccccc3)c3ccccc3)ccc2c1] | 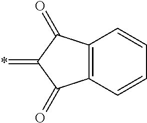 [SMILES: *=C1C(=O)c2ccccc2C1=O] | 452 | 508 | 183 | 210 | 27 | D | D | NG | B | C |
| Comparative Example 6 | C6 | ″ | 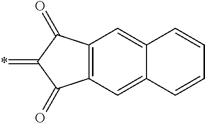 [SMILES: *=C1C(=O)c2cc3ccccc3cc2C1=O] | 502 | 538 | 219 | 247 | 28 | D | C | NG | B | C |
| Comparative Example 7 | C7 | ″ | 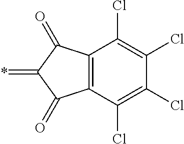 [SMILES: *=C1C(=O)c2c(Cl)c(Cl)c(Cl)c(Cl)c2C1=O] | 589 | 559 | 231 | 278 | 47 | D | B | OK | B | D |
| Comparative Example 8 | C8 | ″ | 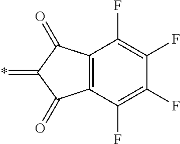 [SMILES: *=C1C(=O)c2c(F)c(F)c(F)c(F)c2C1=O] | 523 | 545 | 201 | 273 | 72 | B | B | NG | D | D |
| Comparative Example 9 | C9 | 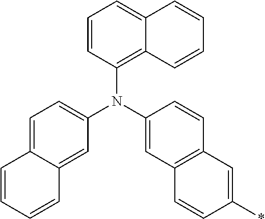 [SMILES: *c1ccc2cc(N(c3ccc4ccccc4c3)c3cccc4ccccc34)ccc2c1] | 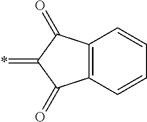 [SMILES: *=C1C(=O)c2ccccc2C1=O] | 552 | 512 | 232 | 253 | 21 | D | D | NG | B | C |
| Comparative Example 10 | C10 | ″ | 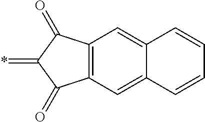 [SMILES: *=C1C(=O)c2cc3ccccc3cc2C1=O] | 601 | 539 | 270 | 300 | 30 | D | C | OK | AA | B |
| Comparative Example 11 | C11 | 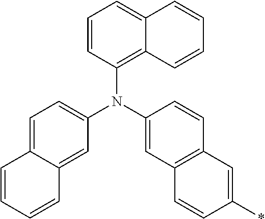 [SMILES: *c1ccc2cc(N(c3ccc4ccccc4c3)c3cccc4ccccc34)ccc2c1] | 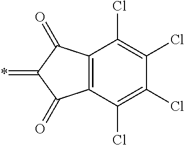 [SMILES: *=C1C(=O)c2c(Cl)c(Cl)c(Cl)c(Cl)c2C1=O] | 689 | 553 | 285 | 322 | 37 | D | B | OK | B | C |
| Comparative Example 12 | C12 | 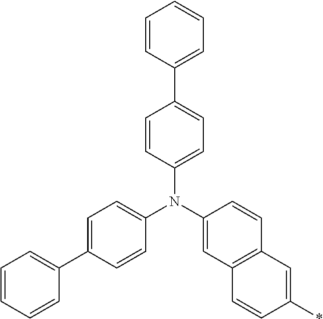 [SMILES: *c1ccc2cc(N(c3ccc(-c4ccccc4)cc3)c3ccc(-c4ccccc4)cc3)ccc2c1] | 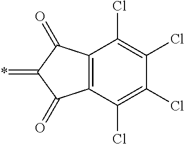 [SMILES: *=C1C(=O)c2c(Cl)c(Cl)c(Cl)c(Cl)c2C1=O] | 741 | 563 | 275 | 283 | 8 | D | A | OK | C | C |
| Comparative Example 13 | C13 | 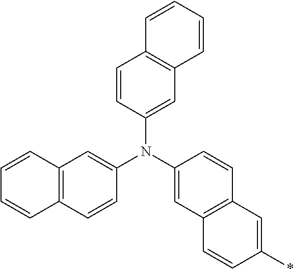 [SMILES: *c1ccc2cc(N(c3ccc4ccccc4c3)c3ccc4ccccc4c3)ccc2c1] | 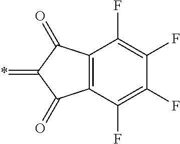 [SMILES: *=C1C(=O)c2c(F)c(F)c(F)c(F)c2C1=O] | 624 | 554 | 261 | 315 | 54 | D | B | OK | D | D |
| Comparative Example 14 | C14 | 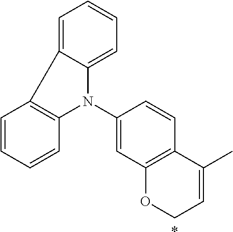 | 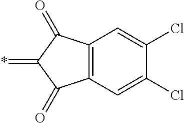 [SMILES: *=C1C(=O)c2cc(Cl)c(Cl)cc2C1=O] | 522 | 453 | 265 | 301 | 36 | D | D | OK | D | D |

As shown in Table 1, the photoelectric conversion element of the present invention exhibits heat resistance, a high photoelectric conversion efficiency, a low level of dark currents, rapid response, and sensitivity characteristics to red. Moreover, the compound used for the element has excellent vapor deposition characteristics, and can be subjected to continuous vapor deposition processing under a high-temperature condition.

For example, while Compound D1 of Example 1 did not exhibit deterioration of the purity even after the compound underwent 5 hours of continuous vapor deposition, Compound C10 of Comparative example 10 was practically completely degraded.

Moreover, λmax of Compound D9 of Example 9 was 580 nm that was longer 26 nm than 554 nm as λmax of Compound C7 which was a non-condensed ring and had no halogen group introduced into the compound. Regarding this point, IPCE spectra of the prepared elements were compared to each other, and as a result, it was confirmed that sensitivity of Compound D9 actually increased around 650 nm of red.

Furthermore, as shown in Table 1, it was found that the introduction of a condensed ring structure and a halogen group into the structure of the compound improves heat resistance of the compound. In Compounds D1 to D40, performance deterioration of the element having undergone thermal annealing was not observed. On the contrary, as shown in comparative examples, in Compounds C1, C2, C5, C6, and C9 which were non-condensed rings and had no halogen introduced into the compounds, decrease in the external quantum efficiency and increase in the dark currents were observed.

Moreover, from the comparison between Example 7 and Example 16 (or 17), it was confirmed that when Z₁ is a group represented by Formula (Z1), vapor deposition characteristic of the compound are further improved.

Furthermore, from the comparison between Example 7 and Example 23 (or 24), it was confirmed that when Ar₁ is a substituted or unsubstituted divalent arylene group, various characteristics of the compound are further improved.

In addition, from the comparison among Example 7 to Example 14, it was confirmed that when Z₁ is represented by Formula (Z2), if the substituent is a chloro group among halogen groups, the compounds are more excellent from the viewpoint of responsiveness and vapor deposition characteristic.

Moreover, from the comparison between Example 13 and Example 15, it was confirmed that when the substituent is a halogen group, the responsiveness and photoelectric conversion efficiency are further improved, compared to a case where the substituent is a halogenated alkyl group.

Furthermore, from the comparison among Examples 1 to 3, it was confirmed that when the compounds are substituted with two halogen groups or two halogenated alkyl groups, vapor deposition characteristic and the like are further improved.

In addition, from the comparison between Example 19 and Example 32 and the comparison between Example 7 and Example 33, it was confirmed that when a halogen group is introduced into a donor site, responsiveness of the compound is further improved when the halogen group is a fluorine group, compared to a case where the halogen group is a chloro group.

<Preparation of Imaging Device>

An imaging device in the same form as shown in FIG. 2 was prepared. That is, on a CMOS substrate, amorphous TiN was formed into a film of 30 nm by a sputtering method, and then a lower electrode was formed by performing patterning by photolithography such that each photodiode (PD) on the CMOS substrate had one pixel. Thereafter, the process from the formation of a film of an electron blocking material was performed according to the same procedure as in Examples 1 to 40, thereby preparing an imaging device. The imaging device was evaluated in the same manner as described above, and the same results as shown in Table 1 were obtained. Consequentially, it was found that the compounds are appropriate for producing an imaging device and show excellent performance.

What is claimed is:

1. A photoelectric conversion element in which a conductive film, a photoelectric conversion film containing a photoelectric conversion material, and a transparent conductive film are laminated on one another in this order,
wherein the photoelectric conversion material includes a compound represented by Formula (1). 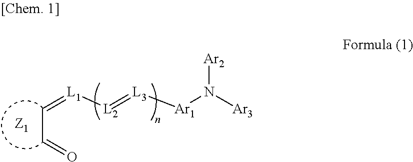
(In Formula (1), Z₁ is a ring which contains at least two carbon atoms and may have a substituent, and represents a 5-membered ring, a 6-membered ring, or a condensed ring which contains at least one of 5-membered ring and 6-membered ring. Each of L₁, L₂, and L₃ independently represents a substituted or unsubstituted methine group. n represents an integer equal to or greater than 0. Ar₁ represents a substituted or unsubstituted divalent arylene group or a substituted or unsubstituted divalent heteroarylene group. Each of Ar₂ and Ar₃ independently represents a substituted or unsubstituted aryl group or a substituted or unsubstituted heteroaryl group. Ar₁ and Ar₂, Ar₁ and Ar₃, and Ar₂ and Ar₃ may respectively form a ring by being bonded to each other, and at least either Ar₁ and Ar₂ or Ar₁ and Ar₃ forms a ring by being bonded to each other. Ar₁ and L₁ may form a ring by being bonded to each other, and the ring may have a substituent. At least one of Z₁, Ar₁, Ar₂, and Ar₃ is substituted with a halogen group or a halogenated alkyl group.)

2. The photoelectric conversion element according to claim 1,
wherein Z₁ is a group represented by Formula (Z1). 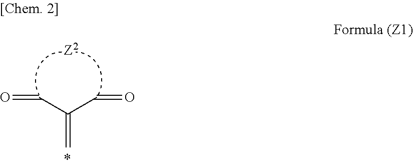
(In Formula (Z1), Z₂ is a ring containing at least three carbon atoms, and represents a 5-membered ring, a 6-membered ring, or a condensed ring which contains at least one of 5-membered ring and 6-membered ring. * represents a binding site where Z₂ is bonded to L₁ in Formula (1).

3. The photoelectric conversion element according to claim 1,
wherein Ar₁ is a substituted or unsubstituted divalent arylene group.

4. The photoelectric conversion element according to claim 1,
wherein the halogen group or a halogen group contained in the halogenated alkyl group is a chloro group or a fluorine group.

5. The photoelectric conversion element according to claim 1,
wherein at least one of Z₁, Ar₁, Ar₂, and Ar₃ is substituted with a halogen group.

6. The photoelectric conversion element according to claim 1,
wherein at least one of Z₁, Ar₁, Ar₂, and Ar₃ is substituted with one to two halogen groups or halogenated alkyl groups.

7. The photoelectric conversion element according to claim 1,
wherein the compound represented by Formula (1) is a compound represented by Formula (2). 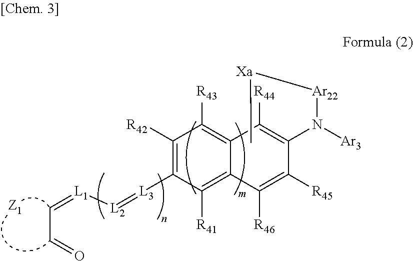
(In Formula (2), Z₁ is a ring which contains at least two carbon atoms and may have a substituent, and represents a 5-membered ring, a 6-membered ring, or a condensed ring which contains least one of 5-membered ring and 6-membered ring. Each of L₁, L₂, and L₃ independently represents a substituted or unsubstituted methine group. n represents an integer equal to or greater than 0. Ar₂₂ represents a substituted or unsubstituted divalent arylene group or a substituted or unsubstituted divalent heteroarylene group. Ar₃ represents a substituted or unsubstituted aryl group or a substituted or unsubstituted heteroaryl group. Each of R₄₁ to R₄₆ independently represents a hydrogen atom or a substituent. R₄₂ and R₄₃, R₄₃ and R₄₄, R₄₅ and R₄₆, and R₄₁ and R₄₆ may respectively form a ring by being bonded to each other. Xa represents a single bond, an oxygen atom, a sulfur atom, an alkylene group, a silylene group, an alkenylene group, a cycloalkylene group, a cycloalkenylene group, an arylene group, a divalent heterocyclic group, or an imino group, and these may further have a substituent. Xa forms a link instead of one of R₄₁ to R₄₆. Ar₂₂ and Ar₃ as well as Ar₃ and R₄₁ to R₄₆ may respectively form a ring by being bonded to each other. m represents 0 or 1. At least one of Z₁, Ar₂₂, and Ar₃ is substituted with a halogen group or a halogenated alkyl group, or alternatively, at least one of R₄₁ to R₄₆ is a halogen group or a halogenated alkyl group. As another option, Xa has substituents, and at least one of the substituents is a halogen group or a halogenated alkyl group.)

8. The photoelectric conversion element according to claim 7,
wherein at least one of Ar₂₂ and Ar₃ in Formula (2) is substituted with a halogen group.

9. The photoelectric conversion element according to claim 8,
wherein the halogen group is a fluorine group.

10. The photoelectric conversion element according to claim 1,
wherein the photoelectric conversion film further contains an n-type organic compound.

11. The photoelectric conversion element according to claim 10,
wherein the n-type organic compound contains fullerene or derivatives thereof.

12. The photoelectric conversion element according to claim 11,
wherein a ratio of the content of the fullerene or derivatives thereof to a total content of the fullerene or derivatives thereof and the compound represented by Formula (1) (a film thickness expressed in terms of a single layer of the fullerene or derivatives thereof/(a film thickness expressed in terms of a single layer of the compound represented by Formula (1)+a film thickness expressed in terms of a single layer of the fullerene or derivatives thereof)) is equal to or higher than 50% by volume.

13. The photoelectric conversion element according to claim 1,
wherein a charge blocking layer is disposed between the conductive film and the transparent conductive film.

14. The photoelectric conversion element according to claim 1,
wherein the photoelectric conversion film is formed by a vacuum vapor deposition method.

15. The photoelectric conversion element according to claim 1,
wherein light enters the photoelectric conversion film through the transparent conductive film.

16. The photoelectric conversion element according to claim 1,
wherein the transparent conductive film is formed of a transparent conductive metal oxide.

17. An imaging device comprising the photoelectric conversion element according to claim 1.

18. A photosensor comprising the photoelectric conversion element according to claim 1.

19. A method for using the photoelectric conversion element according to claim 1,
wherein the conductive film and the transparent conductive film form a pair of electrodes, and an electric field of 1×10⁻⁴ V/cm to 1×10⁷ V/cm is applied between the pair of electrodes.

20. A compound represented by Formula (1) 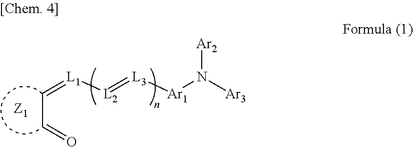
(In Formula (1), Z₁ is a ring which contains at least two carbon atoms and may have a substituent, and represents a 5-membered ring, a 6-membered ring, or a condensed ring which contains at least one of 5-membered ring and 6-membered ring. Each of L₁, L₂, and L₃ independently represents a substituted or unsubstituted methine group. n represents an integer equal to or greater than 0. Ar₁ represents a substituted or unsubstituted divalent arylene group or a substituted or unsubstituted divalent heteroarylene group. Each of Ar₂ and Ar₃ independently represents a substituted or unsubstituted aryl group or a substituted or unsubstituted heteroaryl group. Ar₁ and Ar₂, Ar₁ and Ar₃, and Ar₂ and Ar₃ may respectively form a ring by being bonded to each other, and at least either Ar₁ and Ar₂ or Ar₁ and Ar₃ forms a ring by being bonded to each other. Ar₁ and L₁ may form a ring by being bonded to each other, and the ring may have a substituent. At least one of Z₁, Ar₁, Ar₂, and Ar₃ is substituted with a halogen group or a halogenated alkyl group.)
